SEQUENCE LISTING (7 sequences)

SEQ ID NO: 1 - protein, 1745 aa, Homo sapiens
MGNRRDLGQPRAGLCLLLAALQLLPGTQADPVDVLKALGVQGGQAGVPEGPGFCPQRTPEGDRAFRIGQASTLGIPTWELFPEGHFPENFSLLITLRGQPANQSVLLSIYDERGARQLGLALGPALGLLGDPFRPLPQQVNLTDGRWHRVAVSIDGEMVTLVADCEAQPPVLGHGPRFISIAGLTVLGTQDLGEKTFEGDIQELLISPDPQAAFQACERYLPDCDNLAPAATVAPQGEPETPRPRRKGKGKGRKKGRGRKGKGRKKNKEIWTSSPPPDSAENQTSTDIPKTETPAPNLPPTPTPLVVTSTVTTGLNATILERSLDPDSGTELGTLETKAAREDEEGDDSTMGPDFRAAEYPSRTQFQIFPGAGEKGAKGEPAVIEKGQQFEGPPGAPGPQGVVGPSGPPGPPGFPGDPGPPGPAGLPGIPGIDGIRGPPGTVIMMPFQFAGGSFKGPPVSFQQAQAQAVLQQTQLSMKGPPGPVGLTGRPGPVGLPGHPGLKGEEGAEGPQGPRGLQGPHGPPGRVGKMGRPGADGARGLPGDTGPKGDRGFDGLPGLPGEKGQRGDFGHVGQPGPPGEDGERGAEGPPGPTGQAGEPGPRGLLGPRGSPGPTGRPGVTGIDGAPGAKGNVGPPGEPGPPGQQGNHGSQGLPGPQGLIGTPGEKGPPGNPGIPGLPGSDGPLGHPGHEGPTGEKGAQGPPGSAGPPGYPGPRGVKGTSGNRGLQGEKGEKGEDGFPGFKGDVGLKGDQGKPGAPGPRGEDGPEGPKGQAGQAGEEGPPGSAGEKGKLGVPGLPGYPGRPGPKGSIGFPGPLGPIGEKGKSGKTGQPGLEGERGPPGSRGERGQPGATGQPGPKGDVGQDGAPGIPGEKGLPGLQGPPGFPGPKGPPGHQGKDGRPGHPGQRGELGFQGQTGPPGPAGVLGPQGKTGEVGPLGERGPPGPPGPPGEQGLPGLEGREGAKGELGPPGPLGKEGPAGLRGFPGPKGGPGDPGPTGLKGDKGPPGPVGANGSPGERGPLGPAGGIGLPGQSGSEGPVGPAGKKGSRGERGPPGPTGKDGIPGPLGPLGPPGAAGPSGEEGDKGDVGAPGHKGSKGDKGDAGPPGQPGIRGPAGHPGPPGADGAQGRRGPPGLFGQKGDDGVRGFVGVIGPPGLQGLPGPPGEKGEVGDVGSMGPHGAPGPRGPQGPTGSEGTPGLPGGVGQPGAVGEKGERGDAGDPGPPGAPGIPGPKGDIGEKGDSGPSGAAGPPGKKGPPGEDGAKGSVGPTGLPGDLGPPGDPGVSGIDGSPGEKGDPGDVGGPGPPGASGEPGAPGPPGKRGPSGHMGREGREGEKGAKGEPGPDGPPGRTGPMGARGPPGRVGPEGLRGIPGPVGEPGLLGAPGQMGPPGPLGPSGLPGLKGDTGPKGEKGHIGLIGLIGPPGEAGEKGDQGLPGVQGPPGPKGDPGPPGPIGSLGHPGPPGVAGPLGQKGSKGSPGSMGPRGDTGPAGPPGPPGAPAELHGLRRRRRFVPVPLPVVEGGLEEVLASLTSLSLELEQLRRPPGTAERPGLVCHELHRNHPHLPDGEYWIDPNQGCARDSFRVFCNFTAGGETCLYPDKKFEIVKLASWSKEKPGGWYSTFRRGKKFSYVDADGSPVNVVQLNFLKLLSATARQNFTYSCQNAAAWLDEATGDYSHSARFLGTNGEELSFNQTTAATVSVPQDGCRLRKGQTKTLFEFSSSRAGFLPLWDVAATDFGQTNQKFGFELGPVCFSS

SEQ ID NO: 2 - protein, 429 aa, Artificial Sequence
  an artificially synthesized sequence
HMGREGREGEKGAKGEPGPDGPPGRTGPMGARGPPGRVGPEGLRGIPGPVGEPGLLGAPGQMGPPGPLGPSGLPGLKGDTGPKGEKGHIGLIGLIGPPGEAGEKGDQGLPGVQGPPGPKGDPGPPGPIGSLGHPGPPGVAGPLGQKGSKGSPGSMGPRGDTGPAGPPGPPGAPAELHGLRRRRRFVPVPLPVVEGGLEEVLASLTSLSLELEQLRRPPGTAERPGLVCHELHRNHPHLPDGEYWIDPNQGCARDSFRVFCNFTAGGETCLYPDKKFEIVKLASWSKEKPGGWYSTFRRGKKFSYVDADGSPVNVVQLNFLKLLSATARQNFTYSCQNAAAWLDEATGDYSHSARFLGTNGEELSFNQTTAATVSVPQDGCRLRKGQTKTLFEFSSSRAGFLPLWDVAATDFGQTNQKFGFELGPVCFSS

SEQ ID NO: 3 - protein, 1316 aa, Artificial sequnce
  an artificially synthesized sequence
MGNRRDLGQPRAGLCLLLAALQLLPGTQADPVDVLKALGVQGGQAGVPEGPGFCPQRTPEGDRAFRIGQASTLGIPTWELFPEGHFPENFSLLITLRGQPANQSVLLSIYDERGARQLGLALGPALGLLGDPFRPLPQQVNLTDGRWHRVAVSIDGEMVTLVADCEAQPPVLGHGPRFISIAGLTVLGTQDLGEKTFEGDIQELLISPDPQAAFQACERYLPDCDNLAPAATVAPQGEPETPRPRRKGKGKGRKKGRGRKGKGRKKNKEIWTSSPPPDSAENQTSTDIPKTETPAPNLPPTPTPLVVTSTVTTGLNATILERSLDPDSGTELGTLETKAAREDEEGDDSTMGPDFRAAEYPSRTQFQIFPGAGEKGAKGEPAVIEKGQQFEGPPGAPGPQGVVGPSGPPGPPGFPGDPGPPGPAGLPGIPGIDGIRGPPGTVIMMPFQFAGGSFKGPPVSFQQAQAQAVLQQTQLSMKGPPGPVGLTGRPGPVGLPGHPGLKGEEGAEGPQGPRGLQGPHGPPGRVGKMGRPGADGARGLPGDTGPKGDRGFDGLPGLPGEKGQRGDFGHVGQPGPPGEDGERGAEGPPGPTGQAGEPGPRGLLGPRGSPGPTGRPGVTGIDGAPGAKGNVGPPGEPGPPGQQGNHGSQGLPGPQGLIGTPGEKGPPGNPGIPGLPGSDGPLGHPGHEGPTGEKGAQGPPGSAGPPGYPGPRGVKGTSGNRGLQGEKGEKGEDGFPGFKGDVGLKGDQGKPGAPGPRGEDGPEGPKGQAGQAGEEGPPGSAGEKGKLGVPGLPGYPGRPGPKGSIGFPGPLGPIGEKGKSGKTGQPGLEGERGPPGSRGERGQPGATGQPGPKGDVGQDGAPGIPGEKGLPGLQGPPGFPGPKGPPGHQGKDGRPGHPGQRGELGFQGQTGPPGPAGVLGPQGKTGEVGPLGERGPPGPPGPPGEQGLPGLEGREGAKGELGPPGPLGKEGPAGLRGFPGPKGGPGDPGPTGLKGDKGPPGPVGANGSPGERGPLGPAGGIGLPGQSGSEGPVGPAGKKGSRGERGPPGPTGKDGIPGPLGPLGPPGAAGPSGEEGDKGDVGAPGHKGSKGDKGDAGPPGQPGIRGPAGHPGPPGADGAQGRRGPPGLFGQKGDDGVRGFVGVIGPPGLQGLPGPPGEKGEVGDVGSMGPHGAPGPRGPQGPTGSEGTPGLPGGVGQPGAVGEKGERGDAGDPGPPGAPGIPGPKGDIGEKGDSGPSGAAGPPGKKGPPGEDGAKGSVGPTGLPGDLGPPGDPGVSGIDGSPGEKGDPGDVGGPGPPGASGEPGAPGPPGKRGPSG

SEQ ID NO: 4 - protein, 10 aa, Artificial sequnce
  an artificially synthesized sequence
HMGREGREGE

SEQ ID NO: 5 - protein, 11 aa, Artificial sequnce
  an artificially synthesized sequence
GHMGREGREGE

SEQ ID NO: 6 - protein, 10 aa, Artificial sequnce
  an artificially synthesized sequence
GPPGKRGPSG

SEQ ID NO: 7 - protein, 11 aa, Artificial sequnce
  an artificially synthesized sequence
GPPGKRGPSGH

(19) United States
(12) Patent Application Publication
(10) Pub. No.: US 2022/0221472 A1
(43) Pub. Date: Jul. 14, 2022

(54) METHOD FOR DIAGNOSING ENDOMETRIOSIS, DISEASE STATE MONITORING METHOD, AND KIT

(71) Applicants: Chugai Seiyaku Kabushiki Kaisha, Tokyo (JP); JIchi Medical University, Tokyo (JP); National Institutes of Biomedical Innovation, Health and Nutrition, Osaka (JP)

(72) Inventors: Shinta KOBAYASHI, Kanagawa (JP); Kohji NAGANO, Kanagawa (JP); Hiroshi OHMORI, Kanagawa (JP); Ayako KAKIUCHI, Tokyo (JP); Ryo KONNO, Saitama (JP); Tadashi SANKAI, Ibaraki (JP)

(21) Appl. No.: 17/605,656

(22) PCT Filed: Apr. 24, 2020
(86) PCT No.: PCT/JP2020/017595

§ 371 Date: Oct. 22, 2021

(30) Foreign Application Priority Data
Apr. 26, 2019 (JP) 2019-086223

(51) Int. Cl. G01N 33/68

(52) U.S. Cl. CPC G01N 33/6893; G01N 2800/7052; G01N 2800/56; G01N 2800/364

(57) ABSTRACT

It has been discovered that plasma samples from endometriosis patients contain a number of markers whose abundance is different from those in healthy individuals. It has also been discovered that, by measuring the abundance of those markers, whether or not a subject is affected by endometriosis can be diagnosed, the extent of endometrial fibrosis or adhesion in a subject can be determined, pain of a patient affected or suspected of being affected by endometriosis can be predicted, and pathological conditions of the patient can be monitored.

### TECHNICAL FIELD

The present disclosure relates to methods of diagnosing endometriosis or methods of determining whether or not a subject is affected by endometriosis, methods of determining the extent of endometrial fibrosis or adhesion in a subject affected by endometriosis, methods of predicting pain of a subject affected by endometriosis, methods of monitoring pathological conditions of a subject affected by endometriosis, and the like, and kits and the like for performing these methods.

### BACKGROUND ART

Endometriosis is an estrogen-dependent inflammatory disease observed in 6-10% of women capable of pregnancy, and infertility or pelvic pain is observed in more than 50% of patients (NPL 1). Various hypotheses have been proposed for the pathogenic mechanism of endometriosis (NPL 2) and the relationship with various factors including inflammatory cytokines and chemokines, growth factors, and hormones has been reported (NPL 3). Drugs that target hormones have already been developed, and hormone agents such as GnRH antagonists and progesterone formulations have been shown to alleviate pain and improve pathological conditions (NPLs 4 and 5). However, the strong side effects and difficulty in long-term use of hormone agent treatment present therapeutic problems (NPL 6). Meanwhile, drugs targeting inflammatory cytokines and chemokines are under development and anti-IL-8 antibodies have shown a strong effect of alleviating pathological conditions in a simian endometriosis model (PTL 1).

In the diagnosis of endometriosis, a definitive diagnosis can only be carried out by invasive means such as endoscopic observation or laparotomy. Furthermore, it is difficult for patients to notice the difference between symptoms associated with regular menstruation (e.g., pain) and symptoms of early endometriosis. As such, the hurdle to diagnosing a group of candidate patients is high and it is considered to require 7 to 11 years on average from onset to definitive diagnosis. The delay in definitive diagnosis leads to a delay in the period to start treatment of endometriosis, which is a great clinical problem (NPLs 7 and 8). Therefore, non-invasive diagnostic methods with less burden for patients, such as blood markers, are strongly desired, but precise diagnostic methods have not been established (NPL 9).

### CITATION LIST

### Patent Literature

[PTL 1] WO2018/025982

### Non-Patent Literature

[NPL 1] Giudice L C. Endometriosis. N Engl J Med 2010; 362:2389-98.

[NPL 2] Rogers P A et al. Research priorities for endometriosis. Reprod Sci 2017; 24:202-226.

[NPL 3] Beste M T et al. Molecular network analysis of endometriosis reveals a role for c-Jun-regulated macrophage activation. Sci Transl Med. 2014; 6:222ra216.

[NPL 4] Taylor H S et al. Treatment of Endometriosis-Associated Pain with Elagolix, an Oral GnRH Antagonist. N Engl J Med. 2017; 377:28-40.

[NPL 5] Kohler G et al. A dose-ranging study to determine the efficacy and safety of 1, 2, and 4 mg of dienogest daily for endometriosis. Int J Gynaecol Obstet. 2010; 108:21-5.

[NPL 6] Treatment of Endometriosis-Associated Pain with Elagolix, an Oral GnRH Antagonist.

[NPL 7] Greene R et al. Diagnostic experience among 4,334 women reporting surgically diagnosed endometriosis. Fertility and Sterility, 2009; 91:32-39.

[NPL 8] Manderson L et al. Circuit breaking: Pathways of treatment seeking for women with endometriosis in Australia. Qualitative Health Research, 2008; 18:522-534.

[NPL 9] Fassbender A et al. Update on biomarkers for the detection of endometriosis. Biomed Res Int 2015; 2015:130854.

### SUMMARY OF INVENTION

### Technical Problem

The present disclosure was achieved in view of the above circumstances. An objective of the present disclosure is to provide methods of diagnosing endometriosis or methods of determining whether or not a subject is affected by endometriosis, methods of determining the extent of endometrial fibrosis or adhesion in a subject affected by endometriosis, methods of predicting pain of a subject affected by endometriosis, methods of monitoring pathological conditions of a subject affected by endometriosis, and the like, and kits for performing these methods.

### Solution to Problem

As a result of conducting dedicated research on methods of diagnosing and methods of monitoring pathological conditions of endometriosis, the inventors of the present disclosure have discovered that there are a number of markers whose abundance in patients with endometriosis is different from those in healthy individuals, and also discovered that by measuring the abundance of those markers, whether or not a subject is affected by endometriosis can be diagnosed, the extent of endometrial fibrosis or adhesion can be determined, pain of a subject affected by endometriosis can be predicted, and pathological conditions of a subject affected by endometriosis can be monitored.

The present disclosure is based on these findings and specifically relates to the following inventions:
[1] A method of diagnosing endometriosis, comprising measuring the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B in a sample obtained from a subject.
[2] A method of determining whether or not a subject is affected by endometriosis, comprising measuring the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B in a sample obtained from the subject.
[3] The method of [1] or [2], further comprising a step in which if the abundance of at least one marker selected from the group consisting of the type V collagen MMP degradation product, the markers shown in Table 1A, the markers shown in Table 2A, the markers shown in Table 5A, and the markers shown in Table 6A is high or the abundance of at least one marker selected from the group consisting of the markers shown in Table 1B, the markers shown in Table 2B, the markers shown in Table 5B, and the markers shown in Table 6B is low in the sample obtained from the subject, the subject that the sample is derived from is shown to be affected or potentially affected by endometriosis.
[4] A method of determining the extent of endometrial fibrosis in a subject, comprising measuring the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B in a sample obtained from the subject.
[5] The method of [4], further comprising a step in which if the abundance of at least one marker selected from the group consisting of the type V collagen MMP degradation product, the markers shown in Table 1A, the markers shown in Table 2A, the markers shown in Table 5A, and the markers shown in Table 6A is high or the abundance of at least one marker selected from the group consisting of the markers shown in Table 1B, the markers shown in Table 2B, the markers shown in Table 5B, and the markers shown in Table 6B is low in the sample obtained from the subject, it is shown that endometrial fibrosis has occurred or potentially occurred in the subject that the sample is derived from.
[6] A method of determining the extent of endometrial adhesion in a subject, comprising measuring the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B in a sample obtained from the subject.
[7] The method of [6], further comprising a step in which if the abundance of at least one marker selected from the group consisting of the type V collagen MMP degradation product, the markers shown in Table 1A, the markers shown in Table 2A, the markers shown in Table 5A, and the markers shown in Table 6A is high or the abundance of at least one marker selected from the group consisting of the markers shown in Table 1B, the markers shown in Table 2B, the markers shown in Table 5B, and the markers shown in Table 6B is low in the sample obtained from the subject, it is shown that endometrial adhesion has occurred or potentially occurred in the subject that the sample is derived from.
[8] A method of predicting the degree of pain due to endometriosis in a subject affected or suspected of being affected by endometriosis, comprising measuring the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B in a sample obtained from the subject.
[9] The method of [8], further comprising a step in which if the abundance of at least one marker selected from the group consisting of the type V collagen MMP degradation product, the markers shown in Table 1A, the markers shown in Table 2A, the markers shown in Table 5A, and the markers shown in Table 6A is high or the abundance of at least one marker selected from the group consisting of the markers shown in Table 1B, the markers shown in Table 2B, the markers shown in Table 5B, and the markers shown in Table 6B is low in the sample obtained from the subject, the subject that the sample is derived from is shown to develop or potentially develop pain due to endometriosis.
[10] A method of determining the degree of progression of endometriosis in a subject affected or suspected of being affected by endometriosis, comprising measuring the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B in a sample obtained from the subject.
[11] The method of [10], further comprising a step in which if the abundance of at least one marker selected from the group consisting of the type V collagen MMP degradation product, the markers shown in Table 1A, the markers shown in Table 2A, the markers shown in Table 5A, and the markers shown in Table 6A is high or the abundance of at least one marker selected from the group consisting of the markers shown in Table 1B, the markers shown in Table 2B, the markers shown in Table 5B, and the markers shown in Table 6B is low in the sample obtained from the subject, it is shown that endometriosis is progressing or potentially progressing in the subject that the sample is derived from.
[12] The method of [3], [5], [7], [9], or [11], wherein when the abundance of at least one marker selected from the group consisting of the type V collagen MMP degradation product, the markers shown in Table 1A, the markers shown in Table 2A, the markers shown in Table 5A, and the markers shown in Table 6A is higher than the abundance of the same marker in a sample obtained from a healthy individual, the abundance is determined to be high.
[13] The method of [3], [5], [7], [9], or [11], wherein when the abundance of at least one marker selected from the group consisting of the type V collagen MMP degradation product, the markers shown in Table 1A, the markers shown in Table 2A, and the markers shown in Table 5A is two-fold or higher than the abundance of the same marker in a sample obtained from a healthy individual and/or when the abundance of at least one marker selected from the markers shown in Table 6A is 1.6-fold or higher than the abundance of the same marker in a sample obtained from a healthy individual, the abundance is determined to be high.
[14] The method of [3], [5], [7], [9], or [11], wherein when the abundance of at least one marker selected from the group consisting of the markers shown in Table 1B, the markers shown in Table 2B, the markers shown in Table 5B, and the markers shown in Table 6B is lower than the abundance of the same marker in a sample obtained from a healthy individual, the abundance is determined to be low.
[15] The method of [3], [5], [7], [9], or [11], wherein when the abundance of at least one marker selected from the group consisting of the markers shown in Table 1B, the markers shown in Table 2B, and the markers shown in Table 5B is 0.5-fold or lower than the abundance of the same marker in a sample obtained from a healthy individual and/or when the abundance of at least one marker selected from the markers shown in Table 6B is 0.625-fold or lower than the abundance of the same marker in a sample obtained from a healthy individual, the abundance is determined to be low.
[16] A method of monitoring pathological conditions of endometriosis in a subject affected or suspected of being affected by endometriosis, comprising measuring each of the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B in each of a plurality of samples collected from the subject at different points of time.
[17] The method of [16], further comprising a step in which if the abundance of at least one marker selected from the group consisting of the type V collagen MMP degradation product, the markers shown in Table 1A, the markers shown in Table 2A, the markers shown in Table 5A, and the markers shown in Table 6A decreases over time or the abundance of at least one marker selected from the group consisting of the markers shown in Table 1B, the markers shown in Table 2B, the markers shown in Table 5B, and the markers shown in Table 6B increases over time, it is shown that the pathological conditions of endometriosis are improving or potentially improving in the subject that the sample is derived from.
[18] The method of [16], further comprising a step in which if the abundance of at least one marker selected from the group consisting of the type V collagen MMP degradation product, the markers shown in Table 1A, the markers shown in Table 2A, the markers shown in Table 5A, and the markers shown in Table 6A increases over time or the abundance of at least one marker selected from the group consisting of the markers shown in Table 1B, the markers shown in Table 2B, the markers shown in Table 5B, and the markers shown in Table 6B decreases over time, it is shown that the pathological conditions of endometriosis are worsening or potentially worsening in the subject that the sample is derived from.
[19] The method of any one of [1] to [18], wherein the marker is measured as a polypeptide.
[20] The method of any one of [1] to [19], wherein the sample is a blood sample.
[21] The method of [20], wherein the abundance of the marker in the sample is a concentration of a polypeptide of the marker in the blood sample.
[21-1] The method of [20] or [21], wherein the concentration of the type V collagen MMP degradation product in the blood sample is measured by specifically recognizing the peptide represented by SEQ ID NO: 4 or SEQ ID NO: 6.
[22] A kit for diagnosing endometriosis, comprising a reagent for measuring the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B.
[23] A kit for determining whether or not a subject is affected by endometriosis, comprising a reagent for measuring the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B.
[24] The kit of [22] or [23], further comprising instructions stating that if the abundance of at least one marker selected from the group consisting of the type V collagen MMP degradation product, the markers shown in Table 1A, the markers shown in Table 2A, the markers shown in Table 5A, and the markers shown in Table 6A is high or the abundance of at least one marker selected from the group consisting of the markers shown in Table 1B, the markers shown in Table 2B, the markers shown in Table 5B, and the markers shown in Table 6B is low in a sample obtained from a subject, the subject that the sample is derived from is shown to be affected or potentially affected by endometriosis.
[25] A kit for determining the extent of endometrial fibrosis in a subject, comprising a reagent for measuring the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B.
[26] The kit of [25], further comprising instructions stating that if the abundance of at least one marker selected from the group consisting of the type V collagen MMP degradation product, the markers shown in Table 1A, the markers shown in Table 2A, the markers shown in Table 5A, and the markers shown in Table 6A is high or the abundance of at least one marker selected from the group consisting of the markers shown in Table 1B, the markers shown in Table 2B, the markers shown in Table 5B, and the markers shown in Table 6B is low in a sample obtained from a subject, it is shown that endometrial fibrosis has occurred or potentially occurred in the subject that the sample is derived from.
[27] A kit for determining the extent of endometrial adhesion in a subject, comprising a reagent for measuring the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B.
[28] The kit of [27], further comprising instructions stating that if the abundance of at least one marker selected from the group consisting of the type V collagen MMP degradation product, the markers shown in Table 1A, the markers shown in Table 2A, the markers shown in Table 5A, and the markers shown in Table 6A is high or the abundance of at least one marker selected from the group consisting of the markers shown in Table 1B, the markers shown in Table 2B, the markers shown in Table 5B, and the markers shown in Table 6B is low in a sample obtained from a subject, it is shown that endometrial adhesion has occurred or potentially occurred in the subject that the sample is derived from.
[29] A kit for predicting the degree of pain due to endometriosis in a subject affected or suspected of being affected by endometriosis, comprising a reagent for measuring the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B.
[30] The kit of [29], further comprising instructions stating that if the abundance of at least one marker selected from the group consisting of the type V collagen MMP degradation product, the markers shown in Table 1A, the markers shown in Table 2A, the markers shown in Table 5A, and the markers shown in Table 6A is high or the abundance of at least one marker selected from the group consisting of the markers shown in Table 1B, the markers shown in Table 2B, the markers shown in Table 5B, and the markers shown in Table 6B is low in a sample obtained from a subject, the subject that the sample is derived from is shown to develop or potentially develop pain due to endometriosis.
[31] A kit for determining the degree of progression of endometriosis in a subject affected or suspected of being affected by endometriosis, comprising a reagent for measuring the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B.
[32] The kit of [31], further comprising instructions stating that if the abundance of at least one marker selected from the group consisting of the type V collagen MMP degradation product, the markers shown in Table 1A, the markers shown in Table 2A, the markers shown in Table 5A, and the markers shown in Table 6A is high or the abundance of at least one marker selected from the group consisting of the markers shown in Table 1B, the markers shown in Table 2B, the markers shown in Table 5B, and the markers shown in Table 6B is low in a sample obtained from a subject, it is shown that endometriosis is progressing or potentially progressing in the subject that the sample is derived from.
[33] The kit of [24], [26], [28], [30], or [32], wherein if the abundance of at least one marker selected from the group consisting of the type V collagen MMP degradation product, the markers shown in Table 1A, the markers shown in Table 2A, the markers shown in Table 5A, and the markers shown in Table 6A is higher than the abundance of the same marker in a sample obtained from a healthy individual, the abundance is determined to be high.
[34] The kit of [24], [26], [28], [30], or [32], wherein if the abundance of at least one marker selected from the group consisting of the type V collagen MMP degradation product, the markers shown in Table 1A, the markers shown in Table 2A, and the markers shown in Table 5A is two-fold or higher than the abundance of the same marker in a sample obtained from a healthy individual and/or if the abundance of at least one marker selected from the markers shown in Table 6A is 1.6-fold or higher than the abundance of the same marker in a sample obtained from a healthy individual, the abundance is determined to be high.
[35] The kit of [24], [26], [28], [30], or [32], wherein if the abundance of at least one marker selected from the group consisting of the markers shown in Table 1B, the markers shown in Table 2B, the markers shown in Table 5B, and the markers shown in Table 6B is lower than the abundance of the same marker in a sample obtained from a healthy individual, the abundance is determined to be low.
[36] The kit of [24], [26], [28], [30], or [32], wherein if the abundance of at least one marker selected from the group consisting of the markers shown in Table 1B, the markers shown in Table 2B, and the markers shown in Table 5B that is 0.5-fold or lower than the abundance of the same marker present in a sample obtained from a healthy individual and/or the abundance of at least one marker selected from the markers shown in Table 6B that is 0.625-fold or lower than the abundance of the same marker in a sample obtained from a healthy individual, the abundance is determined to be low.
[37] A kit for monitoring pathological conditions of endometriosis in a subject affected or suspected of being affected by endometriosis, comprising a reagent for measuring the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B.
[38] The kit of [37], further comprising instructions stating that the abundance of at least one marker selected from the group consisting of the type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B in each of a plurality of samples collected from the subject at different points of time is compared.
[39] The kit of [37] or [38], further comprising instructions stating that if the abundance of at least one marker selected from the group consisting of the type V collagen MMP degradation product, the markers shown in Table 1A, the markers shown in Table 2A, the markers shown in Table 5A, and the markers shown in Table 6A decreases over time or the abundance of at least one marker selected from the group consisting of the markers shown in Table 1B, the markers shown in Table 2B, the markers shown in Table 5B, and the markers shown in Table 6B increases over time, it is shown that the pathological conditions of endometriosis are improving or potentially improving in the subject that the sample is derived from.
[40] The kit of [37] or [38], further comprising instructions stating that if the abundance of at least one marker selected from the group consisting of the type V collagen MMP degradation product, the markers shown in Table 1A, the markers shown in Table 2A, the markers shown in Table 5A, and the markers shown in Table 6A increases over time or the abundance of at least one marker selected from the group consisting of the markers shown in Table 1B, the markers shown in Table 2B, the markers shown in Table 5B, and the markers shown in Table 6B decreases over time, it is shown that the pathological conditions of endometriosis are worsening or potentially worsening in the subject that the sample is derived from.
[41] The kit of any one of [22] to [40], wherein the marker is measured as a polypeptide.
[42] The kit of any one of [22] to [41], wherein the sample is a blood sample.
[43] The kit of [42], wherein the abundance of the marker in the sample is a concentration of a polypeptide of the marker in the blood sample.
[43-1] The kit of [42] or [43], wherein the concentration of the type V collagen MMP degradation product in the blood sample is measured by specifically recognizing the peptide represented by SEQ ID NO: 4 or SEQ ID NO: 6.
[44] A reagent for measuring the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B, for use in the diagnosis of endometriosis.
[45] A reagent for measuring the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B, for use in the determination of whether or not a subject is affected by endometriosis.
[46] A reagent for measuring the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B, for use in the determination of the extent of endometrial fibrosis in a subject.
[47] A reagent for measuring the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B, for use in the determination of the extent of endometrial adhesion in a subject.
[48] A reagent for measuring the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B, for use in the prediction of the degree of pain due to endometriosis in a subject affected or suspected of being affected by endometriosis.
[49] A reagent for measuring the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B, for use in the determination of the degree of progression of endometriosis in a subject affected or suspected of being affected by endometriosis.
[50] A reagent for measuring the abundance of at least one marker selected from the group consisting of a type V collagen MMP degradation product, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B, for use in the monitoring of pathological conditions of endometriosis in a subject affected or suspected of being affected by endometriosis.
[51] The reagent of any one of [44] to [50], wherein the marker is measured as a polypeptide.
[52] The reagent of any one of [44] to [51], wherein the abundance of the marker is measured by measuring a concentration of a polypeptide of the marker in a blood sample obtained from the subject.

[53] The reagent of [51], wherein the concentration of the type V collagen MMP degradation product in the blood sample is measured by specifically recognizing the peptide represented by SEQ ID NO: 4 or SEQ ID NO: 6 present in the blood sample.

### BRIEF DESCRIPTION OF DRAWINGS

FIG. 1-1 to FIG. 1-10 show the results of analyzing the relative expression levels of proteins in the plasma of healthy individuals/endometriosis patients by LC-MS. The title of each graph shows Gene Symbol:UniProt ID of the target protein. The X axis represents the name of the sample and the Y axis represents the relative expression level of the protein. The bars in the graph represent the standard deviation of the relative expression level of the protein in three data sets obtained by three analyses. FIG. 1-1 shows the data on Gene Symbol: CALU and UniProt ID: 043852.

FIG. 1-2 shows the data on Gene Symbol: CAT and UniProt ID: P04040.

FIG. 1-3 shows the data on Gene Symbol: HBA1 and UniProt ID: P69905.

FIG. 1-4 shows the data on Gene Symbol: HBB and UniProt ID: P68871.

FIG. 1-5 shows the data on Gene Symbol: HBD and UniProt ID: P02042.

FIG. 1-6 shows the data on Gene Symbol: PPIA and UniProt ID: P62937.

FIG. 1-7 shows the data on Gene Symbol: PRDX1;PRDX4 and UniProt ID: Q06830.

FIG. 1-8 shows the data on Gene Symbol: S100A6 and UniProt ID: P06703.

FIG. 1-9 shows the data on Gene Symbol: TPI1 and UniProt ID: P60174.

FIG. 1-10 shows the data on Gene Symbol: UBE2V1;UBE2V2 and UniProt ID: Q13404.

FIG. 2-1 to FIG. 2-10 show the results of analyzing the expression levels of proteins in the plasma of healthy individuals/endometriosis patients by SOMAscan®. The title of each graph shows Gene Symbol:UniProt ID of the target protein. The X axis represents the name of the sample and the Y axis represents the aptamer signal value of the target protein in SOMAscan®. FIG. 2-1 shows the data on Gene Symbol: BGN and UniProt ID: P21810.

FIG. 2-2 shows the data on Gene Symbol: CDH12 and UniProt ID: P55289.

FIG. 2-3 shows the data on Gene Symbol: DDX19B and UniProt ID: Q9UMR2.

FIG. 2-4 shows the data on Gene Symbol: IL1B and UniProt ID: P01584.

FIG. 2-5 shows the data on Gene Symbol: IL2 and UniProt ID: P60568.

FIG. 2-6 shows the data on Gene Symbol: LAG3 and UniProt ID: P18627.

FIG. 2-7 shows the data on Gene Symbol: MFGE8 and UniProt ID: Q08431.

FIG. 2-8 shows the data on Gene Symbol: PDE5A and UniProt ID: 076074.

FIG. 2-9 shows the data on Gene Symbol: SERPINE2 and UniProt ID: P07093.

FIG. 2-10 shows the data on Gene Symbol: SMPDL3A and UniProt ID: Q92484.

FIG. 3 shows the result of analyzing the expression level of a type V collagen degradation product (C5M) in the plasma of healthy individuals/endometriosis patients. The X axis represents the name of the sample and the Y axis represents the concentration of the target degradation product in the plasma (ng/ml).

### DESCRIPTION OF EMBODIMENTS

### Markers

Markers in the present disclosure can be reworded as biomarkers and refer to specific chemical substances in the body that can be measured and evaluated objectively as indices for normal biological processes, disease development processes, or pharmacological responsiveness to treatment. The markers are useful for evaluating the presence or absence of diseases, progress of diseases, or susceptibility to diseases; evaluating or predicting the effects, the optimal dose, or safety of pharmaceutical agents; predicting prognosis; or such. The markers in the present disclosure are specified by protein names, protein fragment names, or gene names Genes serving as markers are preferably measured as polypeptides or polynucleotides (including the form of DNA and the form of mRNA), and proteins or protein fragments serving as markers are preferably measured as polypeptides.

### Methods of Measuring the Abundance of Markers

The abundance of a marker can be measured by selecting an appropriate method according to the form of the marker or the type of a sample in which the abundance of the marker is to be measured. When the marker is in the form of a polypeptide, it can be measured by immunological methods that use antibodies specifically binding to the polypeptide. Examples of such methods include enzyme immunoassays (ELISA, EIA), fluoroimmunoassays (FIA), radioimmunoassays (RIA), luminescent immunoassays (LIA), electrochemical luminescence (ECL) methods, Western blotting methods, surface plasmon resonance methods, methods that use antibody arrays, immunohistochemical staining methods, fluorescence activated cell sorting (FACS) methods, immunochromatography methods, immunoprecipitation methods, immunonephelometry methods, and latex agglutination methods. When the marker is in the form of a polynucleotide, it can be measured by genetic engineering methods that use oligonucleotides specifically binding to the polynucleotide. Examples of such methods include polymerase chain reaction (PCR) methods, reverse transcription PCR (RT-PCR) methods, real-time quantitative PCR (Q-PCR) methods, northern blotting, and hybridization methods (including methods that use oligonucleotide arrays such as DNA microarrays).

When a marker to be measured is a protein expressed from a gene, the abundance can be reworded as expression level. In the present disclosure, the “abundance” includes the expression level of a protein, the expression level of a gene, and the concentration of a protein fragment.

In an embodiment of the present disclosure, the abundance of a marker can be a relative abundance. A relative abundance can be measured by comparing the level of a specific marker and the level of other proteins/metabolites in a sample obtained from a subject with a control. A relative abundance can also be measured by LC/MS (liquid chromatography/mass spectrometry).

### Criteria for the Abundance of Markers

In the present disclosure, “the abundance of a marker is high or large” means that the measured value of the marker is higher or larger than a predetermined value (control level) for the marker. “The abundance of a marker is low or small” means that it is lower or smaller than a predetermined value (control level) for the marker or not greater than the control level.

The predetermined value in the present disclosure means a value that is predetermined based on a certain scientific basis. It may be any value as long as it can be used as a reference to determine the presence or absence of endometriosis, determine the extent of endometrial fibrosis in a subject, determine the extent of endometrial adhesion in a subject, predict the degree of pain of a subject affected by endometriosis, determine the degree of progression of endometriosis, or monitor pathological conditions of endometriosis. The predetermined value in the present disclosure may be determined for each marker.

The predetermined value in the present disclosure can be determined from the measured value of a marker in a sample (control sample) obtained from a healthy subject, for example, a healthy adult. It has been found in the present disclosure that the measured value of a marker in a sample obtained from a subject affected by endometriosis is increased or decreased compared to the measured value of the marker in a sample obtained from a healthy subject. Thus, one possible approach may be to use as a predetermined value the average of the measured values of a marker in samples obtained from multiple healthy subjects. Another possible approach may be to use as a predetermined value the average of the measured values of a marker in samples obtained from multiple healthy subjects plus a value of 0.1, 0.2, 0.3, 0.4, 0.5, 1.0, 1.5, 2.0, 2.5, or 3.0 times the standard deviation. Accordingly, in one embodiment of the present disclosure, it is shown that determination of whether a test subject is affected by endometriosis, determination of the extent of endometrial fibrosis in a subject, determination of the extent of endometrial adhesion in a subject, prediction of the degree of pain of a subject affected or suspected of being affected by endometriosis, determination of the degree of progression of endometriosis, or monitoring of pathological conditions of endometriosis, is carried out by comparing the abundance of a marker (control level) measured in a sample (control sample) obtained from a healthy individual and the measured value of the marker in a sample obtained from a test subject.

In the present disclosure, the measured values and predetermined values of markers in the present disclosure may be measurement results of the abundance of the markers quantified by any method. In the present disclosure, values obtained as a result of measurement (e.g., color development intensity) may be directly used as measured values or predetermined values of markers, or values converted from measurement results (e.g., concentration) by comparing them with a separately prepared positive control sample that contains a known amount of the marker may be used. Alternatively, values given by, for example, grouping the values obtained as described above into certain intervals and scoring them (e.g., grades 1, 2, and 3) may be used.

### Samples

Samples in the present disclosure can be reworded as biological samples and refer to organs, tissues, cells, body fluids, or mixtures thereof contained in living bodies. Specific examples include skin, respiratory tract, intestinal tract, urogenital tract, nerve, tumor, bone marrow, blood cells, blood (whole blood, plasma, serum), lymph, cerebrospinal fluid, intraperitoneal fluid, synovial fluid, intrapulmonary fluid, saliva, sputum, urine, and such. Samples obtained by washing these or obtained by culturing these ex vivo are also included in the samples of the present disclosure. A preferred sample in the present disclosure is blood, and a particularly preferred sample is plasma or serum.

In the present disclosure, samples obtained from subjects may be processed by methods such as concentration, purification, extraction, isolation, or physical/chemical treatment before subjected to measurement of the abundance of markers. For example, blood cells or plasma components may be isolated from blood samples, and DNA or RNA may be extracted from tissue/cell samples. Alternatively, unwanted components may be denatured/removed by heating or chemical reagents. Such processing is performed mainly for improving the sensitivity and specificity of measurement of the abundance of markers.

In determination of the extent of endometrial fibrosis, determination of the extent of endometrial adhesion, prediction of the degree of pain due to endometriosis, determination of the degree of progression of endometriosis, and monitoring of pathological conditions of endometriosis in the present disclosure, subjects from which samples are obtained may be subjects already diagnosed as being affected by endometriosis or subjects suspected of being affected by endometriosis. Subjects affected by endometriosis may be any subjects as long as they are affected by endometriosis. Subjects may be subjects that have not received, or have already been receiving, treatment for endometriosis.

Subjects in the present disclosure are mammals. Mammals include, but are not limited to, domesticated animals (for example, cows, sheep, cats, dogs, and horses), primates (for example, humans and non-human primates like monkeys), rabbits, and rodents (such as mice and rats). In a certain embodiment, subjects are humans.

### Markers in the Present Disclosure

Markers in the present disclosure include type V collagen MMP (matrix metalloproteinase) degradation products (type V collagen-derived polypeptides comprising the amino acid sequence set forth in SEQ ID NO: 4 at its N-terminus and/or comprising the amino acid sequence set forth in SEQ ID NO: 6 at its C-terminus, such as protein fragments set forth in SEQ ID NOs: 2 and 3), the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B.

TABLE 1A

| UniProt ID | Protein name | Gene name |
|---|---|---|
| P02042 | Hemoglobin subunit delta | HBD |
| P68871 | Hemoglobin subunit beta; | HBB |
| | LVV-hemorphin-7; Spinorphin | |
| P55072 | Transitional endoplasmic reticulum ATPase | VCP |
| P50395; P50395-2 | Rab GDP dissociation inhibitor beta | GDI2 |
| O43852-9; O43852-5; O43852-2; | Calumenin | CALU |
| O43852; O43852-4; O43852-3; | | |
| O43852-12; O43852-13; O43852-14; | | |
| O43852-7; O43852-15; O43852-11; | | |
| O43852-8; O43852-10; O43852-6 | | |
| P04040 | Catalase | CAT |
| Q06830; Q13162 | Peroxiredoxin-1 ; Peroxiredoxin-4 | PRDX1; |
| | | PRDX4 |
| P69905 | Hemoglobin subunit alpha | HBA1 |
| Q13404-8; Q15819; Q13404; | Ubiquitin-conjugating enzyme E2 | UBE2V1; |
| Q13404-7; Q13404-2; Q13404-1 | variant 1; Ubiquitin-conjugating | UBE2V2 |
| | enzyme E2 variant 2 | |
| P06703 | Protein S100-A6 | S100A6 |
| P60174-1; P60174; P60174-4 | Triosephosphate isomerase | TPI1 |
| P62937; P62937-2 | Peptidyl-prolyl cis-trans | PPIA |
| | isomerase A; Peptidyl-prolyl | |
| | cis-trans isomerase A, N-terminally | |
| | processed | |
| P49746-2; P49746 | Thrombospondin-3 | THBS3 |
| P09486 | SPARC | SPARC |
| Q14766-3; Q14766-2; Q14766-5; | Latent-transforming growth factor | LTBP1 |
| Q14766; Q14766-4 | beta-binding protein 1 | |
| P01137 | Transforming growth factor beta-1; | TGFB1 |
| | Latency-associated peptide | |
| P61088; Q5JXB2 | Ubiquitin-conjugating enzyme E2 N; | UBE2N; |
| | Putative ubiquitin-conjugating | UBE2NL |
| | enzyme E2 N-like | |
| P00558-2; P00558 | Phosphoglycerate kinase 1 | PGK1 |
| P07996; P07996-2 | Thrombospondin-1 | THBS1 |
| P02776 | Platelet factor 4; Platelet factor 4, | PF4 |
| | short form | |
| P05067-7; P05067-11; P05067-8; | Amyloid beta A4 protein; N-APP; | APP |
| P05067-9; P05067; P05067-10; | Soluble APP-alpha; Soluble APP-beta; | |
| P05067-3; P05067-4; P05067-5; | C99; Beta-amyloid protein 42; | |
| P05067-6 | Beta-amyloid protein 40; C83; | |
| | P3(42); P3(40); C80; Gamm | |
| | a-secretase C-terminal fragment 59; | |
| | Gamma-secretase C-terminal | |
| | fragment 57; Gamma-secretase | |
| | C-terminal fragment 50; C31 | |
| P00568 | Adenylate kinase isoenzyme 1 | AK1 |
| P00918 | Carbonic anhydrase 2 | CA2 |
| P32119 | Peroxiredoxin-2 | PRDX2 |
| P16070-18; P16070-12; P16070-14; | CD44 antigen | CD44 |
| P16070-13; P16070-11; P16070-10; | | |
| P16070-16; P16070-8; P16070-17; | | |
| P16070-6; P16070-4; P16070-3; | | |
| P16070-7; P16070-5; P16070; | | |
| P16070-15; P16070-9 | | |
| Q13201 | Multimerin-1; Platelet glycoprotein | MMRN1 |
| | la*; 155 kDa platelet multimerin | |
| P27348 | 14-3-3 protein theta | YWHAQ |
| O00592-2; O00592 | Podocalyxin | PODXL |
| P10599-2; P10599 | Thioredoxin | TXN |
| P00915 | Carbonic anhydrase 1 | CA1 |
| P62158 | Calmodulin | CALM1 |
| P58546 | Myotrophin | MTPN |
| P62258; P62258-2 | 14-3-3 protein epsilon | YWHAE |
| O94919 | Endonuclease domain-containing | ENDOD1 |
| | 1 protein | |
| P37837 | Transaldolase | TALDO1 |
| P07738 | Bisphosphoglycerate mutase | BPGM |
| P30043 | Flavin reductase (NADPH) | BLVRB |
| P02775 | Platelet basic protein; Connective | PPBP |
| | tissue-activating peptide III; | |
| | TC-2; Connective tissue-activating | |
| | peptide III(1-81); Beta-thromboglobulin; | |
| | Neutrophil-activating peptide 2(74); | |
| | Neutrophil-activating peptide 2(73); | |
| | Neutrophil-activating peptide 2; TC-1; | |
| | Neutrophil-activating peptide 2(1-66); | |
| | Neutrophil-activating peptide 2(1-63) | |
| P15311 | Ezrin | EZR |
| P0DJI8; P0DJI9-2 | Serum amyloid A-1 protein; | SAA1; |
| | Amyloid protein A; | SAA2 |
| | Serum amyloid protein A(2-104); | |
| | Serum amyloid protein A(3-104); | |
| | Serum amyloid protein A(2-103); | |
| | Serum amyloid protein A(2-102); | |
| | Serum amyloid protein A(4-101); | |
| | Serum amyloid A-2 protein | |
| P10124 | Serglycin | SRGN |
| P13798 | Acylamino-acid-releasing enzyme | APEH |
| P23528 | Cofilin-1 | CFL1 |
| P30041 | Peroxiredoxin-6 | PRDX6 |
| Q8WUM4; Q8WUM4-2 | Programmed cell death | PDCD6IP |
| | 6-interacting protein | |
| P05090 | Apolipoprotein D | APOD |
| Q99497 | Protein deglycase DJ-1 | PARK7 |
| P35579; P35579-2 | Myosin-9 | MYH9 |
| Q13228; Q13228-4; | Selenium-binding protein 1 | SELENBP1 |
| Q13228-2; Q13228-3 | | |
| P13716; P13716-2 | Delta-aminolevulinic acid | ALAD |
| | dehydratase | |
| Q9UL13-2; Q9UL13 | Protein HEG homolog 1 | HEG1 |
| P07384 | Calpain-1 catalytic subunit | CAPN1 |
| P18065 | Insulin-like growth factor-binding | IGFBP2 |
| | protein 2 | |
| P04083 | Annexin A1 | ANXA1 |
| P0DMV8-2; P0DMV9; P0DMV8 | Heat shock 70 kDa protein 1A; | HSPA1A; |
| | Heat shock 70 kDa protein 1B | HSPA1B |
| P07911-3; P07911; P07911-5; | Uromodulin; Uromodulin, secreted | UMOD |
| P07911-4 | form | |
| P15924; P15924-2; P15924-3 | Desmoplakin | DSP |

TABLE 1B

| UniProt ID | Protein name | Gene name |
|---|---|---|
| Q9C0H2-3 | Protein tweety homolog 3 | TTYH3 |

TABLE 2A

| | | Entrez | Entrez Gene |
|---|---|---|---|
| Protein name | UniProt ID | Gene ID | Symbol |
| Pulmonary surfactant-associated | P35247 | 6441 | SFTPD |
| protein D | | | |
| Histone H1.2 | P16403 | 3006 | HIST1H1C |
| Sialic acid-binding Ig-like | Q9Y336 | 27180 | SIGLEC9 |
| lectin 9 | | | |
| Heterogeneous nuclear | P22626 | 3181 | HNRNPA2B1 |
| ribonucleoproteins A2/B1 | | | |
| Hexokinase-2 | P52789 | 3099 | HK2 |
| High mobility group protein B1 | P09429 | 3146 | HMGB1 |
| Thrombospondin-1 | P07996 | 7057 | THBS1 |
| 3-hydroxy-3-methylglutaryl- | P04035 | 3156 | HMGCR |
| coenzyme A reductase | | | |
| Platelet factor 4 | P02776 | 5196 | PF4 |
| Protein S100-A9 | P06702 | 6280 | S100A9 |
| 40S ribosomal protein S3a | P61247 | 6189 | RPS3A |
| Annexin A6 | P08133 | 309 | ANXA6 |
| Inosine-5′-monophosphate | P20839 | 3614 | IMPDH1 |
| dehydrogenase 1 | | | |
| Tyrosine-protein kinase Fgr | P09769 | 2268 | FGR |
| Serine/threonine-protein | O94768 | 9262 | STK17B |
| kinase 17B | | | |
| Neutrophil-activating peptide 2 | P02775 | 5473 | PPBP |
| Estradiol 17-beta-dehydrogenase 1 | P14061 | 3292 | HSD17B1 |
| Connective tissue-activating | P02775 | 5473 | PPBP |
| peptide III | | | |
| Prostaglandin G/H synthase 2 | P35354 | 5743 | PTGS2 |
| Amyloid beta A4 protein | P05067 | 351 | APP |
| GTP-binding nuclear protein Ran | P62826 | 5901 | RAN |
| NAD-dependent protein | Q8IXJ6 | 22933 | SIRT2 |
| deacetylase sirtuin-2 | | | |
| Protein S100-A12 | P80511 | 6283 | S100A12 |
| Plasminogen activator inhibitor 1 | P05121 | 5054 | SERPINE1 |
| SUMO-conjugating enzyme UBC9 | P63279 | 7329 | UBE2I |
| Bactericidal permeability-increasing | P17213 | 671 | BPI |
| protein | | | |
| 6-phosphogluconate | P52209 | 5226 | PGD |
| dehydrogenase, decarboxylating | | | |
| Platelet-derived growth factor | P01127 | 5155 | PDGFB |
| subunit B | | | |
| Tyrosine-protein phosphatase | P29350 | 5777 | PTPN6 |
| non-receptor type 6 | | | |
| Metalloproteinase inhibitor 3 | P35625 | 7078 | TIMP3 |
| NudC domain-containing protein 3 | Q8IVD9 | 23386 | NUDCD3 |
| SPARC | P09486 | 6678 | SPARC |
| Protein 4.1 | P11171 | 2035 | EPB41 |
| Sex hormone-binding globulin | P04278 | 6462 | SHBG |
| Death-associated protein kinase 2 | Q9UIK4 | 23604 | DAPK2 |
| Hepatoma-derived growth | Q7Z4V5 | 84717 | HDGFRP2 |
| factor-related protein 2 | | | |
| Proto-oncogene vav | P15498 | 7409 | VAV1 |
| Serine/threonine-protein kinase | P11309 | 5292 | PIM1 |
| pim-1 | | | |
| Heterogeneous nuclear | Q99729 | 3182 | HNRNPAB |
| ribonucleoprotein A/B | | | |
| Proliferation-associated | Q9UQ80 | 5036 | PA2G4 |
| protein 2G4 | | | |
| Extracellular superoxide | P08294 | 6649 | SOD3 |
| dismutase [Cu-Zn] | | | |
| Angiopoietin-1 | Q15389 | 284 | ANGPT1 |
| Acid sphingomyelinase-like | Q92484 | 10924 | SMPDL3A |
| phosphodiesterase 3a | | | |
| Peroxiredoxin-6 | P30041 | 9588 | PRDX6 |
| AMP Kinase (alpha1beta1gamma1) | Q13131 | 5562 | PRKAA1 |
| | Q9Y478 | 5564 | PRKAB1 |
| | P54619 | 5571 | PRKAG1 |
| Serum amyloid A-1 protein | P0DJI8 | 6288 | SAA1 |
| Insulin-like growth factor-binding | P18065 | 3485 | IGFBP2 |
| \|protein 2 | | | |
| Histone H2B type 2-E | Q16778 | 8349 | HIST2H2BE |
| Fibroblast growth factor 5 | P12034 | 2250 | FGF5 |
| Non-histone chromosomal protein | P05114 | 3150 | HMGN1 |
| HMG-14 | | | |
| Calcium/calmodulin-dependent | Q13557 | 817 | CAMK2D |
| protein kinase type II subunit delta | | | |
| Interstitial collagenase | P03956 | 4312 | MMP1 |
| ICOS ligand | O75144 | 23308 | ICOSLG |
| Calcium/calmodulin-dependent | Q13554 | 816 | CAMK2B |
| protein kinase type II subunit beta | | | |
| Inosine-5′-monophosphate | P12268 | 3615 | IMPDH2 |
| dehydrogenase 2 | | | |
| Dickkopf-related protein 4 | Q9UBT3 | 27121 | DKK4 |
| Glia-derived nexin | P07093 | 5270 | SERPINE2 |
| 14-3-3 protein beta/alpha | P31946 | 7529 | YWHAB |
| Macrophage-capping protein | P40121 | 822 | CAPG |
| ADP-ribosyl cyclase/cyclic | P28907 | 952 | CD38 |
| ADP-ribose hydrolase 1 | | | |
| SH2 domain-containing protein 1A | O60880 | 4068 | SH2D1A |
| Vacuolar protein sorting-associated | Q9NP79 | 51534 | VTA1 |
| protein VTA1 homolog | | | |
| Interleukin-1 beta | P01584 | 3553 | IL1B |
| Chloride intracellular channel | O00299 | 1192 | CLIC1 |
| protein 1 | | | |
| MAP kinase-activated protein | Q16644 | 7867 | MAPKAPK3 |
| kinase 3 | | | |
| Mitogen-activated protein kinase 1 | P28482 | 5594 | MAPK1 |
| Choline/ethanolamine kinase | Q9Y259 | 1120 | CHKB |
| Creatine kinase B-type | P12277 | 1152 | CKB |
| DNA topoisomerase 1 | P11387 | 7150 | TOP1 |
| C-C motif chemokine 5 | P13501 | 6352 | CCL5 |
| C3a anaphylatoxin | P01024 | 718 | C3 |
| Copine-1 | Q99829 | 8904 | CPNE1 |
| Chymase | P23946 | 1215 | CMA1 |
| Mitogen-activated protein kinase | O43318 | 6885 | MAP3K7 |
| kinase kinase 7: TGF-beta-activated | Q15750 | 10454 | TAB1 |
| kinase 1 and MAP3K7-binding | | | |
| protein 1 fusion | | | |
| Dickkopf-related protein 1 | O94907 | 22943 | DKK1 |
| Ephrin type-B receptor 4 | P54760 | 2050 | EPHB4 |
| 40S ribosomal protein S7 | P62081 | 6201 | RPS7 |
| Intercellular adhesion molecule 1 | P05362 | 3383 | ICAM1 |
| Ubiquitin + 1, truncated mutation | P62979 | 6233 | RPS27A |
| for UbB | | | |
| Ubiquitin-conjugating enzyme E2 N | P61088 | 7334 | UBE2N |
| Moesin | P26038 | 4478 | MSN |
| Small ubiquitin-related modifier 3 | P55854 | 6613 | SUMO3 |
| G2/mitotic-specific cyclin-B1 | P14635 | 891 | CCNB1 |
| ATP-dependent RNA helicase | Q9UMR2 | 11269 | DDX19B |
| DDX19B | | | |
| Tyrosine-protein kinase CSK | P41240 | 1445 | CSK |
| Alpha-1-antitrypsin | P01009 | 5265 | SERPINA1 |
| Adenylate kinase isoenzyme 1 | P00568 | 203 | AK1 |
| Mothers against decapentaplegic | P84022 | 4088 | SMAD3 |
| homolog 3 | | | |
| Brain-derived neurotrophic factor | P23560 | 627 | BDNF |
| Atrial natriuretic factor | P01160 | 4878 | NPPA |
| Annexin A1 | P04083 | 301 | ANXA1 |
| Signal transducer and activator of | P42224 | 6772 | STAT1 |
| transcription 1-alpha/beta | | | |
| Angiopoietin-related protein 4 | Q9BY76 | 51129 | ANGPTL4 |
| Cadherin-12 | P55289 | 1010 | CDH12 |
| Stress-induced-phosphoprotein 1 | P31948 | 10963 | STIP1 |
| Glycylpeptide | P30419 | 4836 | NMT1 |
| N-tetradecanoyltransferase 1 | | | |
| Peroxiredoxin-1 | Q06830 | 5052 | PRDX1 |
| Oxidized low-density lipoprotein | P78380 | 4973 | OLR1 |
| receptor 1 | | | |
| VPS10 domain-containing receptor | Q96PQ0 | 57537 | SORCS2 |
| SorCS2 | | | |
| FACT complex subunit SSRP1 | Q08945 | 6749 | SSRP1 |
| Carbonic anhydrase 1 | P00915 | 759 | CA1 |
| Mitogen-activated protein kinase 11 | Q15759 | 5600 | MAPK11 |
| Triosephosphate isomerase | P60174 | 7167 | TPI1 |
| Neurexophilin-1 | P58417 | 30010 | NXPH1 |
| Platelet-derived growth factor | P04085 | 5154 | PDGFA |
| subunit A | | | |
| lnterleukin-22 receptor subunit | Q8N6P7 | 58985 | IL22RA1 |
| alpha-1 | | | |

TABLE 2B

| | | Entrez | Entrez Gene |
|---|---|---|---|
| Protein name | UniProt ID | Gene ID | Symbol |
| Cystatin-SN | P01037 | 1469 | CST1 |
| lnterleukin-22 | Q9GZX6 | 50616 | IL22 |
| Tyrosine-protein kinase Fyn | P06241 | 2534 | FYN |
| Biglycan | P21810 | 633 | BGN |
| Sonic hedgehog protein | Q15465 | 6469 | SHH |
| Cathepsin L2 | 060911 | 1515 | CTSV |
| Interferon alpha/beta receptor 1 | P17181 | 3454 | IFNAR1 |
| Ectodysplasin-A, secreted form | Q92838 | 1896 | EDA |
| Dynein light chain 1, cytoplasmic | P63167 | 8655 | DYNLL1 |
| Cytochrome c | P99999 | 54205 | CYCS |
| Lactoperoxidase | P22079 | 4025 | LPO |
| Tumor necrosis factor receptor | Q969Z4 | 84957 | RELT |
| superfamily member 19L | | | |
| Growth factor receptor-bound protein 2 | P62993 | 2885 | GRB2 |
| Ectonucleotide pyrophosphatase/ | Q6UWV6 | 339221 | ENPP7 |
| phosphodiesterase family member 7 | | | |
| Glucagon | P01275 | 2641 | GCG |
| Eukaryotic translation initiation factor 4 | P78344 | 1982 | EIF4G2 |
| gamma 2 | | | |
| C-X-C motif chemokine 10 | P02778 | 3627 | CXCL10 |
| Platelet-derived growth factor receptor | P09619 | 5159 | PDGFRB |
| beta | | | |
| von Willebrand factor | P04275 | 7450 | VWF |
| Iduronate 2-sulfatase | P22304 | 3423 | IDS |
| cGMP-specific 3′,5′-cyclic | O76074 | 8654 | PDE5A |
| phosphodiesterase | | | |
| Small glutamine-rich tetratricopeptide | O43765 | 6449 | SGTA |
| repeat-containing protein alpha | | | |
| Interleukin-25 | Q9H293 | 64806 | IL25 |
| MHC class I polypeptide-related | Q29980 | 4277 | MICB |
| sequence B | | | |
| C-C motif chemokine 7 | P80098 | 6354 | CCL7 |
| Lactadherin | Q08431 | 4240 | MFGE8 |
| Macrophage metalloelastase | P39900 | 4321 | MMP12 |
| Ubiquitin-like protein ISG15 | P05161 | 9636 | ISG15 |
| Fatty acid-binding protein, liver | P07148 | 2168 | FABP1 |
| Properdin | P27918 | 5199 | CFP |
| Stromelysin-2 | P09238 | 4319 | MMP10 |
| Protein FAM107B | Q9H098 | 83641 | FAM107B |
| Myosin-binding protein C, slow-type | Q00872 | 4604 | MYBPC1 |
| Serine/threonine-protein kinase Chk2 | O96017 | 11200 | CHEK2 |
| Xaa-Pro aminopeptidase 1 | Q9NQW7 | 7511 | XPNPEP1 |
| NT-3 growth factor receptor | Q16288 | 4916 | NTRK3 |
| Interleukin-5 receptor subunit alpha | Q01344 | 3568 | IL5RA |
| Ephrin type-A receptor 2 | P29317 | 1969 | EPHA2 |
| Peptidyl-prolyl cis-trans isomerase F, | P30405 | 10105 | PPIF |
| mitochondrial | | | |
| B-cell lymphoma 6 protein | P41182 | 604 | BCL6 |
| Cell adhesion molecule 1 | Q9BY67 | 23705 | CADM1 |
| Kinesin-like protein KIF23 | Q02241 | 9493 | KIF23 |
| Netrin receptor UNC5D | Q6UXZ4 | 137970 | UNC5D |
| Lymphocyte activation gene 3 protein | P18627 | 3902 | LAG3 |
| Serine protease HTRA2, mitochondrial | O43464 | 27429 | HTRA2 |
| lnterleukin-22 receptor subunit alpha-2 | Q969J5 | 116379 | IL22RA2 |
| Caspase-3 | P42574 | 836 | CASP3 |
| Platelet-derived growth factor receptor | P16234 | 5156 | PDGFRA |
| alpha | | | |
| Interleukin-20 | Q9NYY1 | 50604 | IL20 |
| Ferritin | P02794 | 2495 | FTH1 |
| | P02792 | 2512 | FTL |
| Ephrin-A5 | P52803 | 1946 | EFNA5 |
| Amphoterin-induced protein 2 | Q86SJ2 | 347902 | AMIGO2 |
| Interleukin-2 | P60568 | 3558 | IL2 |
| Phospholipase A2 | P04054 | 5319 | PLA2G1B |
| Protein kinase C alpha type | P17252 | 5578 | PRKCA |
| Tumor necrosis factor ligand | P32971 | 944 | TNFSF8 |
| superfamily member 8 | | | |
| Endoglin | P17813 | 2022 | ENG |
| Gamma-enolase | P09104 | 2026 | ENO2 |
| C-X-C motif chemokine 9 | Q07325 | 4283 | CXCL9 |
| beta-nerve growth factor | P01138 | 4803 | NGF |
| Hepcidin | P81172 | 57817 | HAMP |
| Inorganic pyrophosphatase | Q15181 | 5464 | PPA1 |
| Kallikrein-8 | O60269 | 11202 | KLK8 |
| Semaphorin-6B | Q9H3T3 | 10501 | SEMA6B |
| Adapter molecule crk | P46108 | 1398 | CRK |
| Phosphoglycerate mutase 1 | P18669 | 5223 | PGAM1 |
| Proprotein convertase subtilisin/kexin | Q16549 | 9159 | PCSK7 |
| type 7 | | | |
| Pancreatic hormone | P01298 | 5539 | PPY |
| Interleukin-1 receptor type 1 | P14778 | 3554 | IL1R1 |
| C-X-C motif chemokine 11 | O14625 | 6373 | CXCL11 |
| Secreted frizzled-related protein 1 | Q8N474 | 6422 | SFRP1 |
| Inhibin beta A chain | P08476 | 3624 | INHBA |
| Immunoglobulin D | P01880 | 3495 | IGHD |
| | | 50802 | IGK@ |
| | | 3535 | IGL@ |
| Complement C3b | P01024 | 718 | C3 |
| Teratocarcinoma-derived growth factor 1 | P13385 | 6997 | TDGF1 |
| Brother of CDO | Q9BWV1 | 91653 | BOC |
| CD109 antigen | Q6YHK3 | 135228 | CD109 |
| Aminoacylase-1 | Q03154 | 95 | ACY1 |
| 60 kDa heat shock protein, | P10809 | 3329 | HSPD1 |
| mitochondrial | | | |
| Importin subunit beta-1 | Q14974 | 3837 | KPNB1 |
| C-reactive protein | P02741 | 1401 | CRP |
| Ephrin type-A receptor 1 | P21709 | 2041 | EPHA1 |
| Semaphorin-6A | Q9H2E6 | 57556 | SEMA6A |
| SLIT and NTRK-like protein 5 | O94991 | 26050 | SLITRK5 |
| Leucine-rich repeat transmembrane | Q9NZU0 | 23767 | FLRT3 |
| protein FLRT3 | | | |
| dCTP pyrophosphatase 1 | Q9H773 | 79077 | DCTPP1 |
| Osteopontin | P10451 | 6696 | SPP1 |
| Formimidoyltransferase-cyclodeaminase | O95954 | 10841 | FTCD |
| Ephrin-B2 | P52799 | 1948 | EFNB2 |
| T-lymphocyte surface antigen Ly-9 | Q9HBG7 | 4063 | LY9 |
| Sialic acid-binding Ig-like lectin 14 | Q08ET2 | 100049587 | SIGLEC14 |
| N-acylethanolamine-hydrolyzing acid | Q02083 | 27163 | NAAA |
| amidase | | | |
| Endoplasmic reticulum aminopeptidase 1 | Q9NZ08 | 51752 | ERAP1 |
| Low affinity immunoglobulin gamma Fc | P12318 | 2212 | FCGR2A |
| region receptor II-a | | | |

TABLE 5A

| UniProt ID | Protein name | Gene name |
|---|---|---|
| Q15389 | Angiopoietin-1 (ANG-1) | ANGPT1 |
| P23560 | Brain-Derived Neurotrophic Factor (BDNF) | BDNF |
| O94907 | Dickkopf-related protein 1 (DKK-1) | DKK1 |
| P80511 | S100-A12 | S100A12 |
| P42830 | Epithelial-Derived Neutrophil-Activating | CXCL5 |
| | Protein 78 (ENA-78) | |
| P02794 | Ferritin (FRTN) | FTH1 |
| P09341 | Growth-Regulated alpha protein (GRO-alpha) | CXCL1 |
| P01137 | Latency-Associated Peptide of Transforming | TGFB1 |
| | Growth Factor beta 1 (LAP TGF-b1) | |
| Q8WXL0 | Luteinizing Hormone (LH) | LHB |
| G3CBL7 | MHC class I chain-related protein A (MICA) | MICA |
| P02775 | Platelet basic protein | PPBP |
| P01127 | Platelet-Derived Growth Factor BB (PDGF-BB) | PDGFB |
| P01236 | Prolactin (PRL) | PRL |
| PODJI8; | Serum amyloid A-1 protein; Amyloid protein A; | SAA1; SAA2 |
| PODJI9-2 | Serum amyloid protein A(2-104); Serum amyloid | |
| | protein A(3-104); Serum amyloid protein | |
| | A(2-103); Serum amyloid protein A(2-102); | |
| | Serum amyloid protein A(4-101); Serum | |
| | amyloid A-2 protein | |
| P13501 | T-Cell-Specific Protein RANTES (RANTES) | CCL5 |

TABLE 5B

| UniProt ID | Protein name | Gene name |
|---|---|---|
| P10645 | Chromogranin-A (CgA) | CHGA |
| Q9GZV9 | Fibroblast growth factor 23 (FGF-23) | FGF23 |
| P01266 | Thyroglobul in (TG) | TG |

TABLE 6A

| UniProt ID | Protein name | Gene name |
|---|---|---|
| P07996 | Thrombospondin-1 0S = Homo sapiens 0X = 9606 | THBS1 |
| | GN = THBS1 PE = 1 SV = 2 | |
| P69905 | Hemoglobin subunit alpha 0S = Homo sapiens 0X = 9606 | HBA1 |
| | GN = HBA1 PE = 1 SV = 2 | |
| P32119 | Peroxiredoxin-2 0S = Homo sapiens 0X = 9606 GN = PRDX2 | PRDX2 |
| | PE = 1 SV = 5 | |
| P00918 | Carbonic anhydrase 2 0S = Homo sapiens 0X = 9606 | CA2 |
| | GN = CA2 PE = 1 SV = 2 | |
| P30043 | Flavin reductase (NADPH) 0S = Homo sapiens 0X = 9606 | BLVRB |
| | GN = BLVRB PE = 1 SV = 3 | |
| O76011 | Keratin, type I cuticular Ha4 0S = Homo sapiens | KRT34 |
| | 0X = 9606 GN = KRT34 PE = 1 SV = 2 | |
| P13716 | Delta-aminolevulinic acid dehydratase 0S = | ALAD |
| | Homo sapiens 0X = 9606 GN = ALAD PE = 1 SV = 1 | |
| P00568 | Adenylate kinase isoenzyme 1 0S = Homo sapiens | AK1 |
| | 0X = 9606 GN = AK1 PE = 1 SV = 3 | |
| P07738 | Bisphosphoglycerate mutase 0S = Homo sapiens | BPGM |
| | 0X = 9606 GN = BPGM PE = 1 SV = 2 | |
| P02775 | Platelet basic protein 0S = Homo sapiens 0X = | PPBP |
| | 9606 GN = PPBP PE = 1 SV = 3 | |
| P22392 | Nucleoside diphosphate kinase B 0S = Homo sapiens | NME2 |
| | 0X = 9606 GN = NME2 PE = 1 SV = 1 | |
| P10124 | Serglycin 0S = Homo sapiens 0X = 9606 GN = SRGN | SRGN |
| | PE = 1 SV = 3 | |
| A0A0C4DH67 | Immunoglobulin kappa variable 1-8 0S = | IGKV1-8 |
| | Homo sapiens 0X = 9606 GN = IGKV1-8 PE = 3 SV = 1 | |
| P63241 | Eukaryotic translation initiation factor 5A-1 | EIF5A |
| | 0S = Homo sapiens 0X = 9606 GN = EIF5A PE = 1 SV = 2 | |
| P02776 | Platelet factor 4 0S = Homo sapiens 0X = 9606 | PF4 |
| | GN = PF4 PE = 1 SV = 2 | |
| A0A075B6S2 | Immunoglobulin kappa variable 2D-29 0S = | IGKV2D-29 |
| | Homo sapiens 0X = 9606 GN = IGKV2D-29 PE = 3 SV = 1 | |
| Q9UKU6 | Thyrotropin-releasing hormone-degrading ectoenzyme | TRHDE |
| | 0S = Homo sapiens 0X = 9606 GN = TRHDE PE = 2 SV = 1 | |
| Q8WUM4 | Programmed cell death 6-interacting protein 0S = | PDCD6IP |
| | Homo sapiens 0X = 9606 GN = PDCD6IP PE = 1 SV = 1 | |
| P00441 | Superoxide dismutase [Cu-Zn] 0S = Homo sapiens | SOD1 |
| | 0X = 9606 GN = SOD1 PE = 1 SV = 2 | |

TABLE 6B

| UniProt ID | Protein name | Gene name |
|---|---|---|
| P36980 | Complement factor H-related protein 2 0S = | CFHR2 |
| | Homo sapiens 0X = 9606 GN = CFHR2 PE = 1 SV = 1 | |
| PODOX3 | Immunoglobulin delta heavy chain 0S = Homo sapiens | N/A |
| | 0X = 9606 PE = 1 SV = 1 | |
| P01880 | Immunoglobulin heavy constant delta 0S = | IGHD |
| | Homo sapiens 0X = 9606 GN = IGHD PE = 1 SV = 3 | |
| Q8N6C8 | Leukocyte immunoglobulin-like receptor subfamily A | LILRA3 |
| | member 3 0S = Homo sapiens 0X = 9606 GN = LILRA3 | |
| | PE = 1 SV = 3 | |
| P47929 | Galectin-7 0S = Homo sapiens 0X = 9606 GN = LGALS7 | LGALS7 |
| | PE = 1 SV = 2 | |
| O75339 | Cartilage intermediate layer protein 1 0S = Homo sapiens | CILP |
| | 0X = 9606 GN = CILP PE = 1 SV = 4 | |
| O15389 | Sialic acid-binding Ig-like lectin 5 0S = Homo sapiens | SIGLEC5 |
| | 0X = 9606 GN = SIGLEC5 PE = 1 SV = 1 | |
| P35247 | Pulmonary surfactant-associated protein D 0S = | SFTPD |
| | Homo sapiens 0X = 9606 GN = SFTPD PE = 1 SV = 3 | |

The sequences of the markers (mRNA and protein) listed in these tables can be easily retrieved from the database known to those skilled in the art (Uniprot: https://www.uniprot.org/) on the basis of the Uniprot IDs shown in the above tables. Specifically, a Uniprot ID can be used to search for the corresponding Uniprot entry to retrieve sequences provided in the “sequences” section. It is also possible to obtain IDs/accession numbers for other databases (for example, RefSeq, EMBL, GenBank, DDBJ, CCDS, PIR, UniGene, Ensemble, GeneID, KEGG, and USCS) from the description of the “sequences” section, and retrieve more sequences from those databases. In the present disclosure, the sequence of each marker refers to a sequence and an ID/accession number provided in the latest version of the corresponding Uniprot entry with a release number earlier than 2018-09 (for example, 2018-08; if 2018-08 does not exist, then 2018_07; if neither 2018_08 nor 2018_07 exists, then 2018_06; the same applies to numbers earlier than 2018_06).

Furthermore, in the FTP where the archives of Uniprot release data are stored (ftp://ftp.uniprot.org/pub/databases/uniprot/previous_releases/), the amino acid sequences of the markers in the above tables can also be retrieved from the data included in directory “release-2018_08/knowledgebase” (the “knowledgebase” directory included in “release-2018_08”) by using the Uniprot IDs shown in the tables.

The groups of markers listed in the above tables include markers indicating fibrosis (TGFB1, SPARC, etc.). Furthermore, the groups of markers listed in the above tables include many molecules considered to be derived from platelets. For example, uniprot says that PPBP, THBS1, PF4, SERPINA1, TIMP3, APP, CALU, CASP3, MMRN1, SAA1, SRGN, VWF, and others are involved in platelet degranulation and such. Zhang et al. demonstrated in an in vitro experimental system using endometrial-derived cells that TGFb1 released from activated platelets induced epithelial-mesenchymal transition (EMT) and fibroblast-to-myofibroblast transdifferentiation (FMT), increased collagen production, and induced fibrosis (Molecular and Cellular Endocrinology, 428, 1-16, 2016). This is consistent with the fact that TGFb1 and platelet-derived proteins were selected as markers in the present disclosure, suggesting that changes in these proteins may reflect changes in platelet activation and such.

Type V collagen MMP degradation products are also called MMP mediated type V collagen degradation and are products resulting from degradation of the alpha 3 chain (Refseq: NP_056534.2, SEQ ID NO: 1) of type V collagen by MMP. Specifically, the type V collagen MMP degradation products are type V collagen degradation products produced by cleaving type V collagen shown in SEQ ID NO: 1 between amino acids 1316 (G) and 1317 (H) (Clin Biochem. 2012 May;45(7-8):541-6). Specifically, any polypeptides are included in the type V collagen MMP degradation products as long as they are derived from type V collagen and comprise the amino acid sequence set forth in SEQ ID NO: 4 at the N-terminus and/or comprise the amino acid sequence set forth in SEQ ID NO: 6 at the C-terminus. Examples of the type V collagen MMP degradation products include protein fragments shown in SEQ ID NO: 2 or 3. Among the type V collagen MMP degradation products, the peptide fragment of SEQ ID NO: 4 (HMGREGREGE) is sometimes referred to as C5M.

The presence of a type V collagen MMP degradation product is detected by any method that can detect a type V collagen fragment after cleavage between amino acids 1316 (G) and 1317 (H) set forth in SEQ ID NO: 1. In a certain embodiment, an antibody that specifically recognizes peptide HMGREGREGE (SEQ ID NO: 4) located at the N-terminus of the type V collagen fragment shown in SEQ ID NO: 2 can be used to detect the abundance of a type V collagen MMP degradation product. In a more specific embodiment, an antibody that can recognize peptide HMGREGREGE (SEQ ID NO: 4) located at the N-terminus of the type V collagen fragment shown in SEQ ID NO: 2 and that does not recognize GHMGREGREGE (SEQ ID NO: 5) can be used to detect the abundance of a type V collagen MMP degradation product (an ELISA detection method that uses such an antibody was established in Clin Biochem. 2012 May;45(7-8):541-6.). In another embodiment, an antibody that specifically recognizes peptide GPPGKRGPSG (SEQ ID NO: 6) located at the C-terminus of the type V collagen fragment set forth in SEQ ID NO: 3 can be used to detect the abundance of a type V collagen MMP degradation product. In a more specific embodiment, an antibody that can recognize peptide GPPGKRGPSG (SEQ ID NO: 6) located at the C-terminus of the type V collagen fragment shown in SEQ ID NO: 3 and that does not recognize GPPGKRGPSGH (SEQ ID NO: 7) can be used to detect the abundance of a type V collagen MMP degradation product.

In the present disclosure, a protein selected from the group consisting of type V collagen MMP degradation products, the markers shown in Table 1A, the markers shown in Table 2A, the markers shown in Table 1B, the markers shown in Table 2B, the markers shown in Table 5A, the markers shown in in Table 6A, the markers shown in Table 5B, and the markers shown in Table 6B may be used alone as a marker, or a combination of selected proteins may be used as markers. When more than one protein is used as markers, each of the markers may be measured individually and then the results may be combined, or the markers may be measured together.

The abundance of the markers of the present disclosure can be measured using the measuring methods described in the present disclosure or using commercially available measuring reagents. Specific measured values and predetermined values of each marker described herein can be construed as values measured using the above-mentioned measuring reagents.

The predetermined values (control levels) determined for the markers of the present disclosure can vary depending on the type of a sample of a subject for which the abundance of each marker is measured, but can be determined, for example, from the range of 0.1 to 100 ng/mL. Alternatively, it can be determined from the range of 0.2 to 90 ng/mL, 0.3 to 80 ng/mL, 0.4 to 70 ng/mL, 0.5 to 60 ng/mL, 1.0 to 50 ng/mL, 1.5 to 40 ng/mL, 2.0 to 30 ng/mL, 2.5 to 20 ng/mL, 3.0 to 10 ng/mL, and so on, but is not limited thereto.

The predetermined value determined for each marker of the present disclosure can be determined from values such as 0.1 ng/mL, 0.2 ng/mL, 0.3 ng/mL, 0.4 ng/mL, 0.5 ng/mL, 0.6 ng/mL, 0.7 ng/mL, 0.8 ng/mL, 0.9 ng/mL, 1.0 ng/mL, 1.5 ng/mL, 2.0 ng/mL, 2.5 ng/mL, 3.0 ng/mL, 3.5 ng/mL, 4.0 ng/mL, 4.5 ng/mL, 5.0 ng/mL, 5.5 ng/mL, 6.0 ng/mL, 6.5 ng/mL, 7.0 ng/mL, 7.5 ng/mL, 8.0 ng/mL, 8.5 ng/mL, 9.0 ng/mL, 9.5 ng/mL, 10 ng/mL, 15 ng/mL, 20 ng/mL, 25 ng/mL, 30 ng/mL, 35 ng/mL, 40 ng/mL, 45 ng/mL, 50 ng/mL, 60 ng/mL, 70 ng/mL, 80 ng/mL, 90 ng/mL, and 100 ng/mL, but is not limited thereto.

In the present disclosure, when the abundance of each marker of the present disclosure is greater than the predetermined value (control level), it can be decided that “the abundance is high” or “the abundance is large.” For example, in the present disclosure, it can be decided that “the abundance is high” or “the abundance is large” when, without limitation, the abundance of at least one marker selected from the group consisting of type V collagen MMP degradation products, the markers shown in Table 1A, the markers shown in Table 2A, and the markers shown in Table 5A is, for example, 1.1 times or more, 1.2 times or more, 1.3 times or more, 1.4 times or more, 1.5 times or more, 1.6 times or more, 1.7 times or more, 1.8 times or more, 1.9 times or more, 2.0 times or more, 2.1 times or more, 2.2 times or more, 2.3 times or more, 2.4 times or more, 2.5 times or more, 2.6 times or more, 2.7 times or more, 2.8 times or more, 2.9 times or more, or 3.0 times or more the abundance of the same marker in a sample obtained from a healthy individual, or more. Alternatively, in the present disclosure, it can be decided that “the abundance is high” or “the abundance is large” when, without limitation, the abundance of at least one marker selected from the markers shown in Table 6A is, for example, 1.10 times or more, 1.15 times or more, 1.20 times or more, 1.25 times or more, 1.30 times or more, 1.35 times or more, 1.40 times or more, 1.45 times or more, 1.50 times or more, 1.55 times or more, 1.60 times or more, 1.65 times or more, 1.70 times or more, 1.75 times or more, 1.80 times or more, 1.85 times or more, 1.90 times or more, 1.95 times or more, or 2.00 times or more the abundance of the same marker in a sample obtained from a healthy individual, or more.

In the present disclosure, when the abundance of each marker of the present disclosure is less than the predetermined value (control level), it can be decided that “the abundance is low” or “the abundance is small.” For example, in the present disclosure, it can be decided that “the abundance is low” or “the abundance is small” when, without limitation, the abundance of at least one marker selected from the group consisting of type V collagen MMP degradation products, the markers shown in Table 1B, the markers shown in Table 2B, and the markers shown in Table 5B is, for example, 0.9 times or less, 0.8 times or less, 0.7 times or less, 0.6 times or less, 0.5 times or less, 0.4 times or less, 0.3 times or less, 0.2 times or less, or 0.1 times or less the abundance of the same marker in a sample obtained from a healthy individual, or less. Alternatively, in the present disclosure, it can be decided that “the abundance is low” or “the abundance is small” when, without limitation, the abundance of at least one marker selected from the markers shown in Table 6B is, for example, 0.9 times or less, 0.8 times or less, 0.7 times or less, 0.6 times or less, 0.5 times or less, 0.4 times or less, 0.3 times or less, 0.2 times or less, or 0.1 times or less the abundance of the same marker in a sample obtained from a healthy individual, or less.

### Diagnosis of Endometriosis and Determination of Whether or Not a Subject is Affected by Endometriosis

One aspect of the present disclosure relates to methods of diagnosing endometriosis or methods of determining whether or not a subject is affected by endometriosis. One aspect of the present disclosure also relates to methods of detecting endometriosis or markers thereof. One aspect of the present disclosure also relates to methods of assisting the diagnosis or determination. One aspect of the present disclosure also relates to methods of providing instructions for the diagnosis or determination. One aspect of the present disclosure relates to reagents and kits for use in these methods.

The above respective methods comprise measuring the abundance of at least one marker selected from the group consisting of type V collagen MMP degradation products, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B in a sample obtained from the subject. The methods can also comprise comparing the abundance with a predetermined value (control level).

The above reagents are reagents for measuring the abundance of at least one marker selected from the group consisting of type V collagen MMP degradation products, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B. The above kits contain such reagents.

The above respective methods may comprise a step in which if the abundance of at least one marker selected from the group consisting of type V collagen MMP degradation products, the markers shown in Table 1A, the markers shown in Table 2A, the markers shown in Table 5A, and the markers shown in Table 6A in the sample obtained from the subject is higher than a predetermined value (control level) or the abundance of at least one marker selected from the group consisting of the markers shown in Table 1B, the markers shown in Table 2B, the markers shown in Table 5B, and the markers shown in Table 6B in the sample obtained from the subject is lower than a predetermined value (control level), the subject that the sample is derived from is shown to be affected or potentially affected by endometriosis.

The above respective methods can be performed both in vivo and in vitro, but are preferably performed in vitro.

Furthermore, the above respective methods may also comprise obtaining a sample from the subject.

Moreover, the above respective methods may also comprise treating a subject that has been shown to be affected or potentially affected by endometriosis. The above respective methods may also comprise administering a known therapeutic agent for endometriosis to a subject that has been shown to be affected or potentially affected by endometriosis. Thus, the present invention relates to methods of treating endometriosis in a subject that has been shown to be affected or potentially affected by endometriosis by each of the above methods. Various therapeutic agents for endometriosis are known to those skilled in the art, and examples thereof include, but are not limited to, estrogen/progesterone mixtures, progesterone preparations, GnRH agonists, GnRH antagonists, and danazol.

The above-mentioned kits may further comprise instructions stating that if the abundance of at least one marker selected from the group consisting of type V collagen MMP degradation products, the markers shown in Table 1A, the markers shown in Table 2A, the markers shown in Table 5A, and the markers shown in Table 6A in the sample obtained from the subject is higher than a predetermined value (control level) or if the abundance of at least one marker selected from the group consisting of the markers shown in Table 1B, the markers shown in Table 2B, the markers shown in Table 5B, and the markers shown in Table 6B is lower than a predetermined value (control level), the subject that the sample is derived from is shown to be affected or potentially affected by endometriosis.

### Determination of the Extent of Endometrial Fibrosis in a Subject

One aspect of the present disclosure relates to methods of determining the extent of endometrial fibrosis in a subject, methods of assisting the determination, methods of providing instructions for the determination, and reagents and kits for use in these.

The above respective methods comprise measuring the abundance of at least one marker selected from the group consisting of type V collagen MMP degradation products, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B in a sample obtained from the subject. The methods can also comprise comparing the abundance with a predetermined value (control level).

The above reagents are reagents for measuring the abundance of at least one marker selected from the group consisting of type V collagen MMP degradation products, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B. The above-mentioned kits contain such reagents.

The above respective methods may comprise a step in which if the abundance of at least one marker selected from the group consisting of type V collagen MMP degradation products, the markers shown in Table 1A, the markers shown in Table 2A, the markers shown in Table 5A, and the markers shown in Table 6A in the sample obtained from the subject is higher than a predetermined value (control level) or if the abundance of at least one marker selected from the group consisting of the markers shown in Table 1B, the markers shown in Table 2B, the markers shown in Table 5B, and the markers shown in Table 6B in the sample obtained from the subject is lower than a predetermined value (control level), it is shown that endometrial fibrosis is occurring or potentially occurring in the subject that the sample is derived from.

The above respective methods can be performed both in vivo and in vitro, but is preferably performed in vitro.

Furthermore, the above respective methods may comprise obtaining a sample from the subject.

Moreover, the above respective methods may also comprise treating a subject in whom or which it has been shown that endometrial fibrosis has occurred or potentially occurred. The above respective methods may also comprise administering a known therapeutic agent for suppressing endometrial fibrosis to a subject in whom or which it has been shown that endometrial fibrosis has occurred or potentially occurred. Thus, the present invention relates to methods of suppressing fibrosis in a subject in whom or which it has been shown by the above respective methods that endometrial fibrosis has occurred or potentially occurred. Examples of therapeutic agents for suppressing endometrial fibrosis include the above-mentioned therapeutic agents for endometriosis.

The above kits may further comprise instructions stating that if the abundance of at least one marker selected from the group consisting of type V collagen MMP degradation products, the markers shown in Table 1A, the markers shown in Table 2A, the markers shown in Table 5A, and the markers shown in Table 6A is higher than a predetermined value (control level) or the abundance of at least one marker selected from the group consisting of the markers shown in Table 1B, the markers shown in Table 2B, the markers shown in Table 5B, and the markers shown in Table 6B is lower than a predetermined value (control level) in a sample obtained from the subject, it is shown that endometrial fibrosis has occurred or potentially occurred in the subject that the sample is derived from.

### Determination of the Extent of Endometrial Adhesion in a Subject

One aspect of the present disclosure relates to methods of determining the extent of endometrial adhesion in a subject, methods of assisting the determination, methods of providing instructions for the determination, and reagents and kits for use in these methods.

The above respective methods comprise measuring the abundance of at least one marker selected from the group consisting of type V collagen MMP degradation products, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B in a sample obtained from the subject. The methods can also comprise comparing the abundance with a predetermined value (control level).

The above reagents are reagents for measuring the abundance of at least one marker selected from the group consisting of type V collagen MMP degradation products, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B. The above kits contain such reagents.

The above respective methods may comprise a step in which if the abundance of at least one marker selected from the group consisting of type V collagen MMP degradation products, the markers shown in Table 1A, the markers shown in Table 2A, the markers shown in Table 5A, and the markers shown in Table 6A in a sample obtained from the subject is higher than a predetermined value (control level) or if the abundance of at least one marker selected from the group consisting of the markers shown in Table 1B, the markers shown in Table 2B, the markers shown in Table 5B, and the markers shown in Table 6B in a sample obtained from the subject is lower than a predetermined value (control level), it is shown that endometrial adhesion has occurred or potentially occurred in the subject that the sample is derived from.

The above respective methods can be performed both in vivo and in vitro, but are preferably performed in vitro.

Furthermore, The above respective methods may comprise obtaining a sample from the subject.

Moreover, the above respective methods may also comprise treating a subject in whom or which it has been shown that endometrial adhesion has occurred or potentially occurred. Each of the above methods may also comprise administering a known therapeutic agent for suppressing endometrial adhesion to a subject in whom or which it has been shown that endometrial adhesion has occurred or potentially occurred. Thus, the present invention relates to methods of suppressing endometrial adhesion in a subject in whom or which it has been shown by the above respective methods that endometrial adhesion has occurred or potentially occurred. Examples of therapeutic agents for suppressing endometrial adhesion include the above-mentioned therapeutic agents for endometriosis.

The above kits may further comprise instructions stating that if the abundance of at least one marker selected from the group consisting of the concentration of type V collagen MMP degradation products, the markers shown in Table 1A, the markers shown in Table 2A, the markers shown in Table 5A, and the markers shown in Table 6A is higher than a predetermined value (control level) or the abundance of at least one marker selected from the group consisting of the markers shown in Table 1B, the markers shown in Table 2B, the markers shown in Table 5B, and the markers shown in Table 6B is lower than a predetermined value (control level) in a sample obtained from the subject, it is shown that endometrial adhesion has occurred or potentially occurred in the subject that the sample is derived from.

### Prediction of the Degree of Pain Due to Endometriosis in a Subject Affected or Suspected of Being Affected by Endometriosis

One aspect of the present disclosure relates to methods of predicting the degree of pain due to endometriosis in a subject affected or suspected of being affected by endometriosis, methods of assisting the prediction, methods of providing instructions for the prediction, and reagents and kits for use in these methods.

The above respective methods comprise measuring the abundance of at least one marker selected from the group consisting of type V collagen MMP degradation products, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B in a sample obtained from the subject. The methods can also comprise comparing the abundance with a predetermined value (control level).

The above reagents are reagents for measuring the abundance of at least one marker selected from the group consisting of type V collagen MMP degradation products, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B. The above kits contain such reagents.

The above respective methods may comprise a step in which if the abundance of at least one marker selected from the group consisting of type V collagen MMP degradation products, the markers shown in Table 1A, the markers shown in Table 2A, the markers shown in Table 5A, and the markers shown in Table 6A in a sample obtained from the subject is higher than a predetermined value (control level) or if the abundance of at least one marker selected from the group consisting of the markers shown in Table 1B, the markers shown in Table 2B, the markers shown in Table 5B, and the markers shown in Table 6B in a sample obtained from the subject is lower than a predetermined value (control level), the subject that the sample is derived from is shown to develop or potentially develop pain due to endometriosis.

The above respective methods can be performed both in vivo and in vitro, but is preferably performed in vitro.

Furthermore, the above respective methods may comprise obtaining a sample from the subject.

Moreover, the above respective methods may also comprise alleviating pain due to endometriosis in a subject that has been shown to develop or potentially develop pain due to endometriosis. The above respective methods may also comprise administering a known therapeutic agent for suppressing pain due to endometriosis to a subject that has been shown to develop or potentially develop pain due to endometriosis. Thus, the present invention relates to methods of suppressing pain due to endometriosis in a subject that has been shown to develop or potentially develop pain due to endometriosis by the above respective methods. Examples of therapeutic agents for suppressing pain due to endometriosis include the above-mentioned therapeutic agents for endometriosis.

The above kits may further comprise instructions stating that if the abundance of at least one marker selected from the group consisting of type V collagen MMP degradation products, the markers shown in Table 1A, the markers shown in Table 2A, the markers shown in Table 5A, and the markers shown in Table 6A is higher than a predetermined value (control level) or the abundance of at least one marker selected from the group consisting of the markers shown in Table 1B, the markers shown in Table 2B, the markers shown in Table 5B, and the markers shown in Table 6B is lower than a predetermined value (control level) in a sample obtained from the subject, the subject that the sample is derived from is shown to develop or potentially develop pain due to endometriosis.

### Determination of the Degree of Progression of Endometriosis

One aspect of the present disclosure relates to methods of determining the degree of progression of endometriosis in a subject affected or suspected of being affected by endometriosis, methods of assisting the determination, methods of providing instructions for the determination, and reagents and kits for use in these methods.

The above respective methods comprise measuring the abundance of at least one marker selected from the group consisting of type V collagen MMP degradation products, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B in a sample obtained from the subject. The methods can also comprise comparing the abundance with a predetermined value (control level).

The above reagents are reagents for measuring the abundance of at least one marker selected from the group consisting of type V collagen MMP degradation products, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B. The above kits contain such reagents.

The above respective methods may comprise a step in which if the abundance of at least one marker selected from the group consisting of type V collagen MMP degradation products, the markers shown in Table 1A, the markers shown in Table 2A, the markers shown in Table 5A, and the markers shown in Table 6A in a sample obtained from the subject is higher than a predetermined value (control level) or if the abundance of at least one marker selected from the group consisting of the markers shown in Table 1B, the markers shown in Table 2B, the markers shown in Table 5B, and the markers shown in Table 6B in a sample obtained from the subject is lower than a predetermined value (control level), it is shown that endometriosis is progressing or potentially progressing in the subject that the sample is derived from.

The above respective methods can be performed both in vivo and in vitro, but are preferably performed in vitro.

Furthermore, the above respective methods may comprise obtaining a sample from the subject.

Moreover, the above respective methods may also comprise treating a subject in whom or which it has been shown that endometriosis is progressing or potentially progressing. The above respective methods may also comprise administering a known therapeutic agent for suppressing progression of endometriosis to a subject in whom or which it has been shown that endometriosis is progressing or potentially progressing. Thus, the present invention relates to methods of suppressing progression of endometriosis in a subject in whom or which it has been shown by the above respective methods that endometriosis is progressing or potentially progressing. Examples of therapeutic agents for suppressing progression of endometriosis include the above-mentioned therapeutic agents for endometriosis.

The above kits may further comprise instructions stating that if the abundance of at least one marker selected from the group consisting of type V collagen MMP degradation products, the markers shown in Table 1A, the markers shown in Table 2A, the markers shown in Table 5A, and the markers shown in Table 6A is higher than a predetermined value (control level) or the abundance of at least one marker selected from the group consisting of the markers shown in Table 1B, the markers shown in Table 2B, the markers shown in Table 5B, and the markers shown in Table 6B is lower than a predetermined value (control level) in a sample obtained from the subject, it is shown that endometriosis is progressing or potentially progressing in the subject that the sample is derived from.

### Monitoring of Pathological Conditions of Endometriosis

One aspect of the present disclosure relates to methods of monitoring pathological conditions of endometriosis in a subject affected or suspected of being affected by endometriosis, methods of assisting the monitoring, methods of providing instructions for the monitoring, and reagents and kits for use in these methods. The monitoring in the present disclosure includes evaluating a therapeutic effect and/or providing information on a future therapeutic regimen or therapeutic policy.

The above respective methods comprise measuring the abundance of at least one marker selected from the group consisting of type V collagen MMP degradation products, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B in a sample obtained from the subject.

The above reagents are reagents for measuring the abundance of at least one marker selected from the group consisting of type V collagen MMP degradation products, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B. The above kits contain such reagents.

The above respective methods may comprise measuring each of the abundance of at least one marker selected from the group consisting of type V collagen MMP degradation products, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B in each of a plurality of samples collected from a subject at different points of time. The methods may also comprise comparing the abundance of the marker in each sample in a plurality of samples collected at different points of time. In the present disclosure, the timing and number of times of collecting samples are not limited, and samples can be collected at various points of time before treatment, during treatment, and after treatment as necessary. Samples can also be collected at various timings within each period of before treatment, during treatment, and after treatment. In the present disclosure, “plurality” is not particularly limited, and examples thereof include 2, 3, 4, 5, 6, 7, 8, 9, 10, or more times. The above kits may comprise instructions stating that the abundance of at least one marker selected from the group consisting of type V collagen MMP degradation products, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B in each of a plurality of samples obtained from a subject collected at different points of time is compared.

The above respective methods may comprise a step in which if the abundance of at least one marker selected from the group consisting of type V collagen MMP degradation products, the markers shown in Table 1A, the markers shown in Table 2A, the markers shown in Table 5A, and the markers shown in Table 6A decreased over time or if the abundance of at least one marker selected from the group consisting of the markers shown in Table 1B, the markers shown in Table 2B, the markers shown in Table 5B, and the markers shown in Table 6B increased over time, it is shown that the pathological conditions of endometriosis are improving or potentially improving in the subject that the sample is derived from.

The above respective methods may comprise a step in which if the abundance of at least one marker selected from the group consisting of type V collagen MMP degradation products, the markers shown in Table 1A, the markers shown in Table 2A, the markers shown in Table 5A, and the markers shown in Table 6A increased over time or if the abundance of at least one marker selected from the group consisting of the markers shown in Table 1B, the markers shown in Table 2B, the markers shown in Table 5B, and the markers shown in Table 6B decreased over time, it is shown that the pathological conditions of endometriosis are worsening or potentially worsening in the subject the sample is derived from.

The above respective methods can be performed both in vivo and in vitro, but are preferably performed in vitro.

Furthermore, the above respective methods may comprise obtaining a sample from the subject.

Moreover, the above respective methods may comprise treating a subject in whom or which it has been shown that the pathological conditions of endometriosis are worsening or potentially worsening. The above respective methods may also comprise administering a known therapeutic agent for endometriosis to a subject in whom or which it has been shown that the pathological conditions of endometriosis are worsening or potentially worsening. Thus, the present invention relates to methods of treating endometriosis in a subject in whom or which it has been indicated by each of the above methods that the pathological conditions of endometriosis are worsening or potentially worsening. The above-mentioned agents can be used as therapeutic agents for endometriosis.

The above kits may comprise instructions stating that if the abundance of at least one marker selected from the group consisting of type V collagen MMP degradation products, the markers shown in Table 1A, the markers shown in Table 2A, the markers shown in Table 5A, and the markers shown in Table 6A decreased over time or if the abundance of at least one marker selected from the group consisting of the markers shown in Table 1B, the markers shown in Table 2B, the markers shown in Table 5B, and the markers shown in Table 6B increased over time, it is shown that the pathological conditions of endometriosis are improving or potentially improving in the subject that the sample is derived from.

The above kits may comprise instructions stating that if the abundance of at least one marker selected from the group consisting of type V collagen MMP degradation products, the markers shown in Table 1A, the markers shown in Table 2A, the markers shown in Table 5A, and the markers shown in Table 6A increased over time or if the abundance of at least one marker selected from the group consisting of the markers shown in Table 1B, the markers shown in Table 2B, the markers shown in Table 5B, and the markers shown in Table 6B decreased over time, it is shown that the pathological conditions of endometriosis are worsening or potentially worsening in the subject that the sample is derived from.

### Reagents/Kits

Reagents contained in the kits of the present disclosure are not particularly limited as long as the abundance of markers can be measured. Reagents can be appropriately selected depending on the form of markers. When the form of a marker is a polypeptide, reagents comprising an antibody that specifically binds to the polypeptide are preferable. When the form of a marker is a polynucleotide, reagents comprising an oligonucleotide that specifically binds to the polynucleotide are preferable. In the present disclosure, the antibody or the polynucleotide itself may be a reagent. Polynucleotides in the present disclosure include those in the form of DNA and in the form of mRNA.

Antibodies in the present disclosure may be chimeric antibodies, humanized antibodies, or human antibodies. In some embodiments, antibodies may be multispecific antibodies, e.g., bispecific antibodies. Alternatively, antibodies in the present disclosure may be antibody fragments or modified products thereof. For example, antibody fragments include Fab, F (ab′)2, Fv, or single chain Fv (scFv) in which an H chain Fv and an L chain Fv are linked with an appropriate linker. These antibodies can be obtained by methods well known to those skilled in the art.

Antibodies can be labeled by commonly known methods. Labeling substances known to those skilled in the art, such as fluorescent dyes, enzymes, coenzymes, chemiluminescent substances, and radioactive substances, can be used.

Oligonucleotides in the present disclosure can be any oligonucleotide that specifically hybridizes to at least a portion of a polynucleotide that is a marker in the present disclosure or a complementary strand thereof. The nucleotide sequence of such an oligonucleotide for detecting a polynucleotide is selected from the nucleotide sequence complementary to the sense strand of a marker in the present disclosure. Oligonucleotides typically have a length of at least 15 bp or more.

Oligonucleotides in the present disclosure can also be used after being labeled by commonly known methods. Oligonucleotides in the present disclosure can be produced, for example, by commercially available oligonucleotide synthesizers.

Kits of the present disclosure preferably contain a positive control sample as a reference for measuring the abundance of markers. The positive control sample is not particularly limited as long as the amount of the markers contained therein has been determined in advance, and can be appropriately prepared depending on the form of markers measured by the kits. For example, when the form of a marker is a polypeptide, a positive control sample is preferably a sample comprising a polypeptide prepared by isolating, purifying, and quantifying the same polypeptide as the marker.

In addition to the above, kits of the present invention may include, for example, sterile water, saline, vegetable oil, surfactant, lipid, solubilizer, buffer, protein stabilizer (such as BSA and gelatin), preservative, blocking solution, reaction solution, reaction stop solution, and reagents for treating samples as necessary.

Sets of type V collagen MMP degradation products, the markers shown in Table 1A, the markers shown in Table 2A, the markers shown in Table 1B, the markers shown in Table 2B, the markers shown in Table 5A, the markers shown in Table 6A, the markers shown in Table 5B, and the markers shown in Table 6B are also included in the present disclosure. Such sets can be used for diagnosing endometriosis, determining the presence or absence of endometriosis, determining the extent of endometrial fibrosis or adhesion in patients affected by endometriosis, predicting pain of patients affected by endometriosis, monitoring pathological conditions of patients affected by endometriosis, and such.

All prior art documents cited herein are incorporated herein by reference.

### EXAMPLES

The present disclosure will be further illustrated by the Examples below but is not limited thereto.

### (Example 1) Search for Biomarkers by LC-MS Analysis Using Plasma of Healthy Human Individuals and Patients with Endometriosis

### (1-1) Obtainment of Human Plasma Samples

Plasma from healthy individuals and endometriosis patients was purchased from Proteogenex.

### (1-2) LC-MS Analysis of Plasma Proteins

With the Seppro IgY14 column (Sigma aldrich), highly abundant proteins in each plasma sample were bound to the column and removed by the method recommended by the manufacturer. The proteins in the flow-through fraction that did not bind to the column were precipitated by the methanol/chloroform method and dissolved in 8 M urea/400 mM ammonium bicarbonate solution. The dissolved proteins were reduced and alkylated, and then digested into peptides with lysyl endopeptidase and trypsin. The peptide solution was demineralized with Monospin C18 (GL Science) by the method recommended by the manufacturer.

The peptides were labeled with TMT10plex Mass Tag Labeling Kits and Reagents (ThermoFisher Scientific) (hereinafter, referred to as TMT 10-plex) by the method recommended by the manufacturer. The labeled peptides were fractionated with Pierce™ High pH Reversed-Phase Peptide Fractionation Kit (ThermoFisher Scientific) by the method recommended by the manufacturer. Each fraction was analyzed by the LC-MS analysis system in which Orbitrap fusion Lumos (ThermoFisher scientific) was coupled to the nano-LC system (Ultimate3000, Dionex). The analysis samples included seven samples: the peptides obtained from the plasma of each of three healthy individuals and three endometriosis patients, and a sample (control) prepared by mixing an equal amount of these six peptide solutions. Each sample was analyzed three times, and an independent data set in each analysis was then analyzed (N=3, hereinafter, data set TMT1, 2, and 3).

The proteins in the samples were identified from the raw data of the LC-MS analysis by the MaxQuant software (http://www.biochem.mpg.de/5111795/maxquant), and their relative expression levels were analyzed. The database search for protein identification was performed with the following parameters. Taxonomy: uniprot human, Fixed modification: carbamidomethylation (C), Variable modification: oxidation (M); deamidation (NQ), Acetyl (protein N-term), and FDR (protein, peptide)<1%. The relative expression levels were calculated by the following formula:

$$Relative expression level = \frac{\begin{matrix}Target protein signal in plasma samples \\ ( from patients with endometriosis or healthy individuals )\end{matrix}}{Target protein signal in the control sample}$$

The target protein signal value used in the above formula was calculated using the column of “Reporter intensity corrected” in Protein groups.txt, which is one of the output files of MaxQunat, where for each label of TMT 10-plex, the sum of the intensity was divided by the sum of the intensity of the control sample and multiplied by 1×10¹¹ using pipeline pilot (BIOVIA).

### (1-3) Selection of Biomarker Candidate Molecules

The LC-MS result data were analyzed for each data set of TMT1, 2, and 3 by statistical programming environment R and Microsoft Excel. From the relative expression level of each protein in each of the samples (three healthy individuals and three endometriosis patients), 58 proteins for which altered expression was found in endometriosis patients were selected according to the following criteria for each data set (Table 3A and Table 3B).

$$- \frac{\begin{matrix}The average value of the relative expression level \\ of the protein in three patients with endometriosis\end{matrix}}{\begin{matrix}The average value of the relative expression \\ level of the protein in three healthy individuals\end{matrix}} ≧ 2 or ≦ 0.5$$

The relative expression level of the protein in three healthy individuals and three endometriosis patients was p<0.05 in the Welch t-test.

TABLE 3A

| | | | Relative expression level of protein: | | |
|---|---|---|---|---|---|
| | | | Average value of 3 patients/ | | |
| | | | Average value of 3 healthy individuals | | |
| | | | Data set | Data set | Data set |
| UniProt ID | Protein name | Gene name | TMT1 | TMT2 | TMT3 |
| P02042 | Hemoglobin subunit delta | HBD | 4.848 | 4.12 | 4.27 |
| P68871 | Hemoglobin subunit beta; | HBB | 3.662 | 4.58 | 4.153 |
| | LVV-hemorphin-7; Spinorphin | | | | |
| P55072 | Transitional endoplasmic reticulum ATPase | VCP | 2.915 | 3 | 2.306 |
| P50395; P50395-2 | Rab GDP dissociation inhibitor beta | GDI2 | 2.06 | 2.19 | 1.784 |
| O43852-9; O43852-5; O43852-2; | Calumenin | CALU | 6.081 | 5.24 | 4.619 |
| O43852; O43852-4; O43852-3; | | | | | |
| O43852-12; O43852-13; O43852-14; | | | | | |
| O43852-7; O43852-15; O43852-11; | | | | | |
| O43852-8; O43852-10; O43852-6 | | | | | |
| P04040 | Catalase | CAT | 3.085 | 3.36 | 3.258 |
| Q06830; Q13162 | Peroxiredoxin-1 ; Peroxiredoxin-4 | PRDX1; | 3.006 | 2.87 | 3.052 |
| | | PRDX4 | | | |
| P69905 | Hemoglobin subunit alpha | HBA1 | 4.877 | 5.88 | 5.135 |
| Q13404-8; Q15819; Q13404; | Ubiquitin-conjugating enzyme E2 | UBE2V1; | 3.279 | 2.76 | 2.538 |
| Q13404-7; Q13404-2; Q13404-1 | variant 1; Ubiquitin-conjugating | UBE2V2 | | | |
| | enzyme E2 variant 2 | | | | |
| P06703 | Protein S100-A6 | S100A6 | 4.041 | 4.37 | 4.052 |
| P60174-1; P60174; P60174-4 | Triosephosphate isomerase | TPI1 | 2.433 | 2.71 | 2.574 |
| P62937; P62937-2 | Peptidyl-prolyl cis-trans | PPIA | 2.957 | 2.84 | 2.631 |
| | isomerase A; Peptidyl-prolyl | | | | |
| | cis-trans isomerase A, N-terminally | | | | |
| | processed | | | | |
| P49746-2; P49746 | Thrombospondin-3 | THBS3 | 3.863 | 1.19 | 1.208 |
| P09486 | SPARC | SPARC | 4.177 | 5.05 | 4.681 |
| Q14766-3; Q14766-2; Q14766-5; | Latent-transforming growth factor | LTBP1 | 6.967 | 2.71 | 6.079 |
| Q14766; Q14766-4 | beta-binding protein 1 | | | | |
| P01137 | Transforming growth factor beta-1; | TGFB1 | 4.8 | 5.77 | 2.667 |
| | Latency-associated peptide | | | | |
| P61088; Q5JXB2 | Ubiquitin-conjugating enzyme | UBE2N; | 2.742 | 2.92 | 3.356 |
| | E2 N; Putative ubiquitin-conjugating | UBE2NL | | | |
| | enzyme E2 N-like | | | | |
| P00558-2; P00558 | Phosphoglycerate kinase 1 | PGK1 | 2.278 | 2.02 | 2.685 |
| P07996; P07996-2 | Thrombospondin-1 | THBS1 | 8.012 | 8.27 | 6.402 |
| P02776 | Platelet factor 4; Platelet factor 4, | PF4 | 6.872 | 7.55 | 7.134 |
| | short form | | | | |
| P05067-7; P05067-11 ; P05067-8; | Amyloid beta A4 protein; N-APP; | APP | 5.951 | 3.84 | 5.453 |
| P05067-9; P05067; P05067-10; | Soluble APP-alpha; Soluble | | | | |
| P05067-3; P05067-4; P05067-5; | APP-beta; C99; Beta-amyloid | | | | |
| P05067-6 | protein 42; Beta-amyloid protein | | | | |
| | 40; C83; P3(42); P3(40); C80; | | | | |
| | Gamm a-secretase C-terminal | | | | |
| | fragment 59; Gamma-secretase | | | | |
| | C-terminal fragment 57; | | | | |
| | Gamma-secretase C-terminal | | | | |
| | fragment 50; C31 | | | | |
| P00568 | Adenylate kinase isoenzyme 1 | AK1 | 2.762 | 3.62 | 2.942 |
| P00918 | Carbonic anhydrase 2 | CA2 | 2.721 | 2.78 | 3.387 |
| P32119 | Peroxiredoxin-2 | PRDX2 | 3.697 | 3.66 | 3.939 |
| P16070-18; P16070-12; P16070-14; | CD44 antigen | CD44 | 2.047 | 1.83 | 1.814 |
| P16070-13; P16070-11; P16070-10; | | | | | |
| P16070-16; P16070-8; P16070-17; | | | | | |
| P16070-6; P16070-4; P16070-3; | | | | | |
| P16070-7; P16070-5; P16070; | | | | | |
| P16070-15; P16070-9 | | | | | |
| Q13201 | Multimerin-1 ; Platelet glycoprotein | MMRN1 | 4.569 | 4.27 | 3.898 |
| | la*; 155 kDa platelet multimerin | | | | |
| P27348 | 14-3-3 protein theta | YWHAQ | 1.983 | 2.65 | 1.942 |
| O00592-2; O00592 | Podocalyxin | PODXL | 1.953 | 2.03 | 2.009 |
| P10599-2; P10599 | Thioredoxin | TXN | 2.744 | 1.94 | 2.597 |
| P00915 | Carbonic anhydrase 1 | CA1 | 3.741 | 3.62 | 4.046 |
| P62158 | Calmodulin | CALM1 | 2.102 | 3.05 | 2.229 |
| P58546 | Myotrophin | MTPN | 1.836 | 2.09 | 1.787 |
| P62258; P62258-2 | 14-3-3 protein epsilon | YWHAE | 2.002 | 1.16 | 1.448 |
| O94919 | Endonuclease domain-containing | ENDOD1 | 3.075 | 2.53 | 3.181 |
| | 1 protein | | | | |
| P37837 | Transaldolase | TALDO1 | 2.995 | 1.02 | 0.806 |
| P07738 | Bisphosphoglycerate mutase | BPGM | 2.761 | 3.14 | 2.939 |
| P30043 | Flavin reductase (NADPH) | BLVRB | 2.985 | 3.09 | 2.68 |
| P02775 | Platelet basic protein; Connective | PPBP | 12.41 | 9.69 | 11.867 |
| | tissue-activating peptide III; TC-2; | | | | |
| | Connective tissue-activating | | | | |
| | peptide III(1-81); Beta-thromboglobulin; | | | | |
| | Neutrophil-activating peptide 2(74); | | | | |
| | Neutrophil-activating peptide 2(73); | | | | |
| | Neutrophil-activating peptide 2; | | | | |
| | TC-1; Neutrophil-activating peptide | | | | |
| | 2(1-66); Neutrophil-activating | | | | |
| | peptide 2(1-63) | | | | |
| P15311 | Ezrin | EZR | 2.116 | 2.1 | 2.294 |
| P0DJI8; P0DJI9-2 | Serum amyloid A-1 protein; | SAA1; SAA2 | 3.374 | 4.17 | 3.645 |
| | Amyloid protein A; Serum amyloid | | | | |
| | protein A(2-104); Serum amyloid protein | | | | |
| | A(3-104); Serum amyloid protein | | | | |
| | A(2-103); Serum amyloid protein | | | | |
| | A(2-102); Serum amyloid protein | | | | |
| | A(4-101); Serum amyloid A-2 protein | | | | |
| P10124 | Serglycin | SRGN | 3.41 | 5.68 | 4.664 |
| P13798 | Acylamino-acid-releasing enzyme | APEH | 4.068 | 4.14 | 4.786 |
| P23528 | Cofilin-1 | CFL1 | 2.704 | 2.79 | 2.566 |
| P30041 | Peroxiredoxin-6 | PRDX6 | 2.111 | 2.01 | 1.81 |
| Q8WUM4; Q8WUM4-2 | Programmed cell death | PDCD6IP | 2.152 | 2.36 | 2.291 |
| | 6-interacting protein | | | | |
| P05090 | Apolipoprotein D | APOD | 2.213 | 2.51 | 2.381 |
| Q99497 | Protein deglycase DJ-1 | PARK7 | 3.668 | 2.22 | 2.939 |
| P35579; P35579-2 | Myosin-9 | MYH9 | 2.97 | 2.8 | 2.504 |
| Q13228; Q13228-4; Q13228-2; | Selenium-binding protein 1 | SELENBP1 | 2.032 | 2.33 | 1.843 |
| Q13228-3 | | | | | |
| P13716; P13716-2 | Delta-aminolevulinic acid | ALAD | 1.983 | 2.28 | 2.524 |
| | dehydratase | | | | |
| Q9ULI3-2; Q9ULI3 | Protein HEG homolog 1 | HEG1 | 2.16 | 1.95 | 2.819 |
| P07384 | Calpain-1 catalytic subunit | CAPN1 | 1.752 | 0.97 | 2.358 |
| P18065 | Insulin-like growth factor-binding | IGFBP2 | 2.507 | 2.87 | 2.833 |
| | protein 2 | | | | |
| P04083 | Annexin A1 | ANXA1 | 2.684 | 8.03 | 3.645 |
| P0DMV8-2; P0DMV9; P0DMV8 | Heat shock 70 kDa protein 1A; | HSPA1A; HSPA 1B | 2.188 | 2.3 | 2.203 |
| | Heat shock 70 kDa protein 1B | | | | |
| P07911-3; P07911; P07911-5; | Uromodulin; Uromodulin, secreted form | UMOD | 1.565 | 1.85 | 2.121 |
| P07911-4 | | | | | |
| P15924; P15924-2; P15924-3 | Desmoplakin | DSP | 1.636 | 3.38 | 1.968 |

TABLE 3B

| | | | Relative expression level of protein: | | |
|---|---|---|---|---|---|
| | | | Average value of 3 patients/ | | |
| | | | Average value of 3 healthy individuals | | |
| | | | Data set | Data set | Data set |
| UniProt ID | Protein name | Gene name | TMT1 | TMT2 | TMT3 |
| Q9C0H2-3 | Protein tweety homolog 3 | TTYH3 | 0.511 | 0.619 | 0.5 |

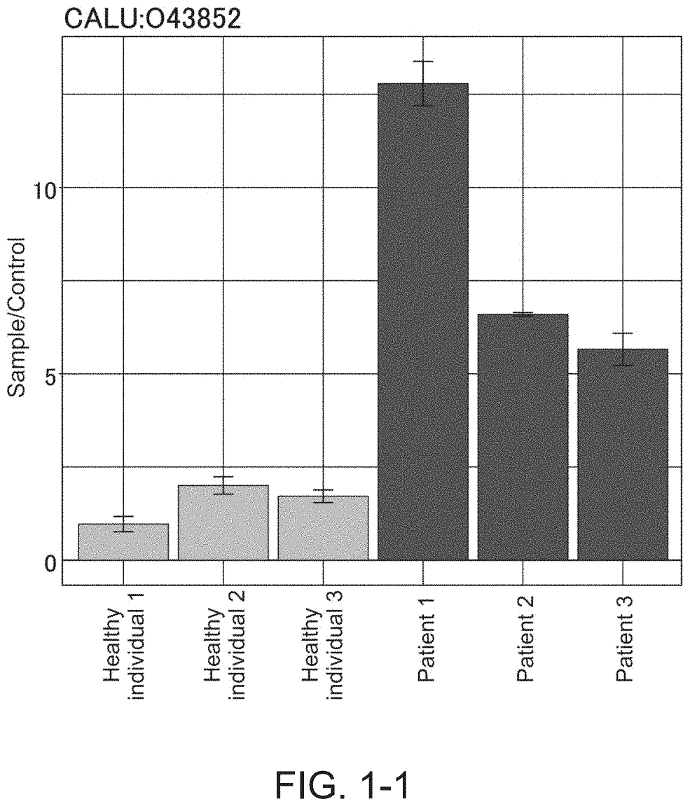

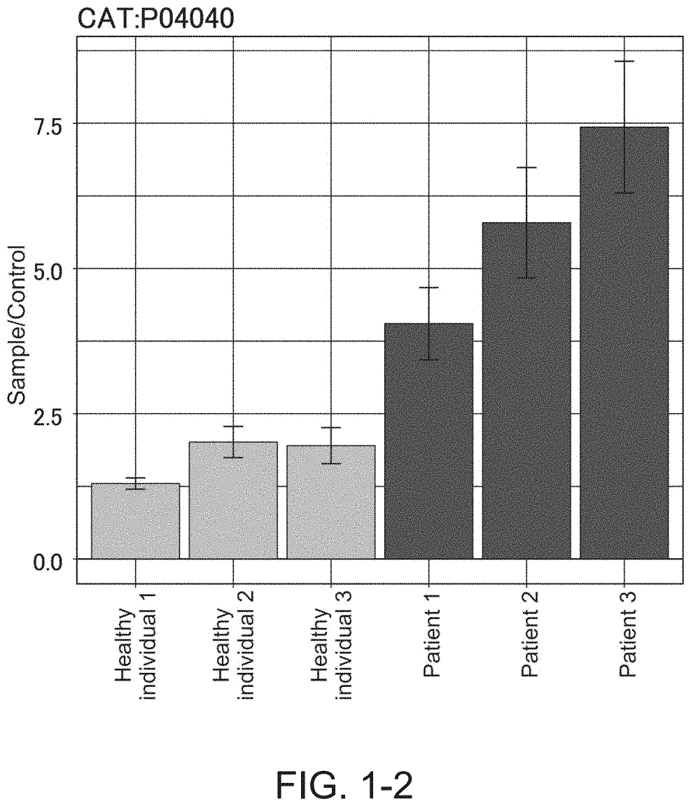

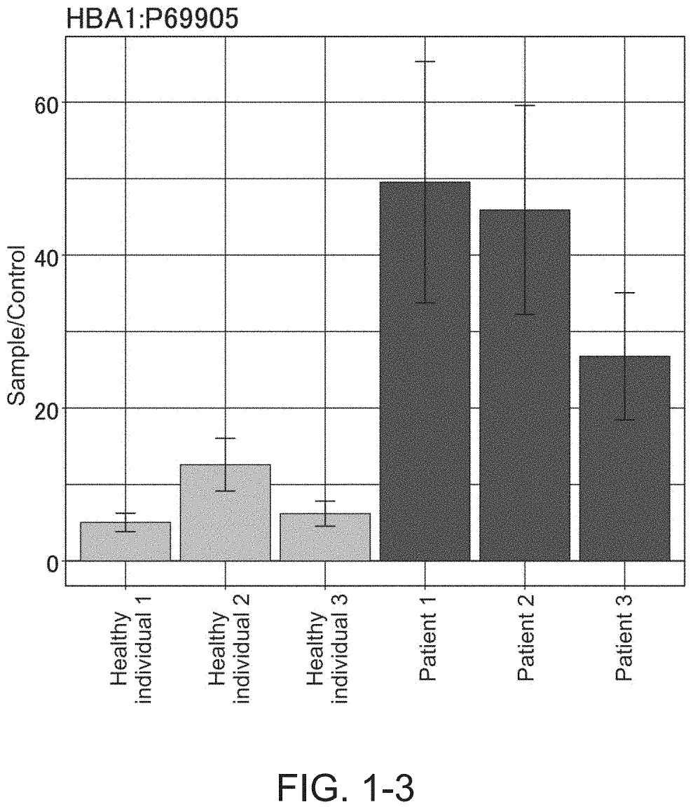

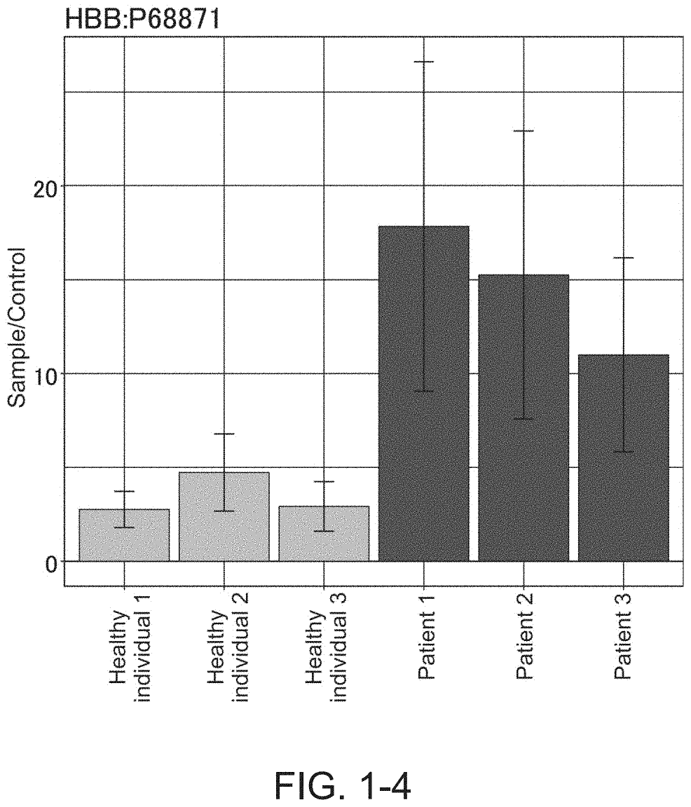

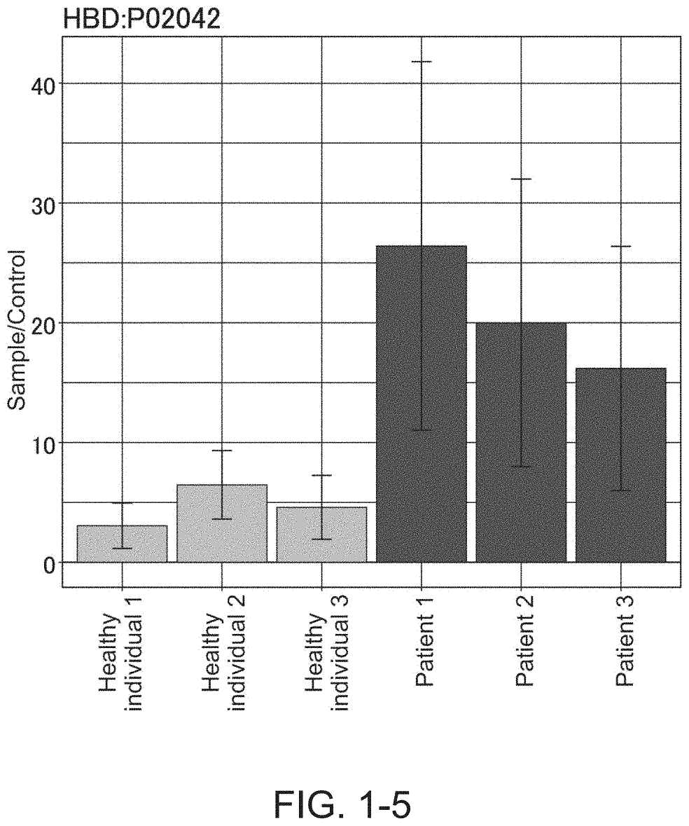

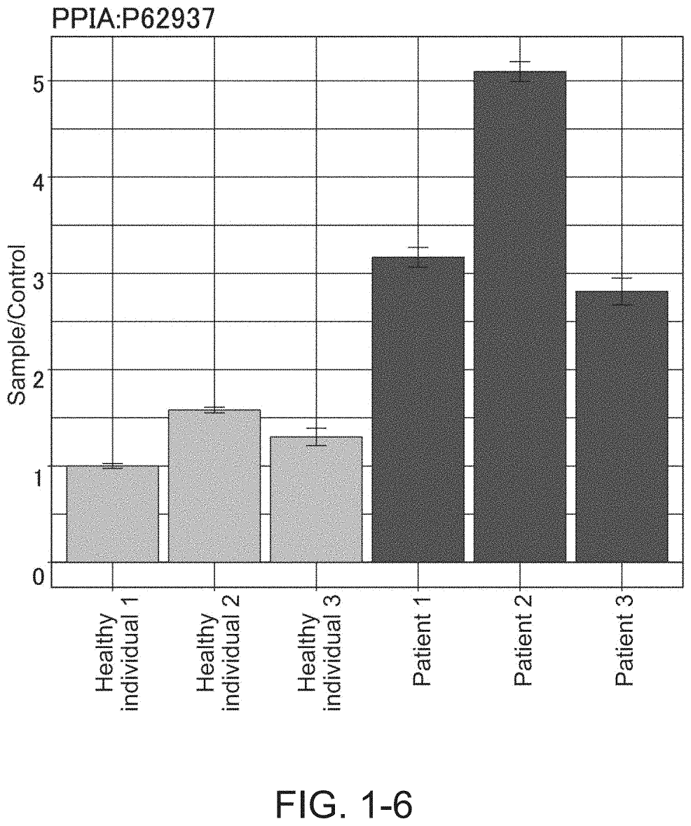

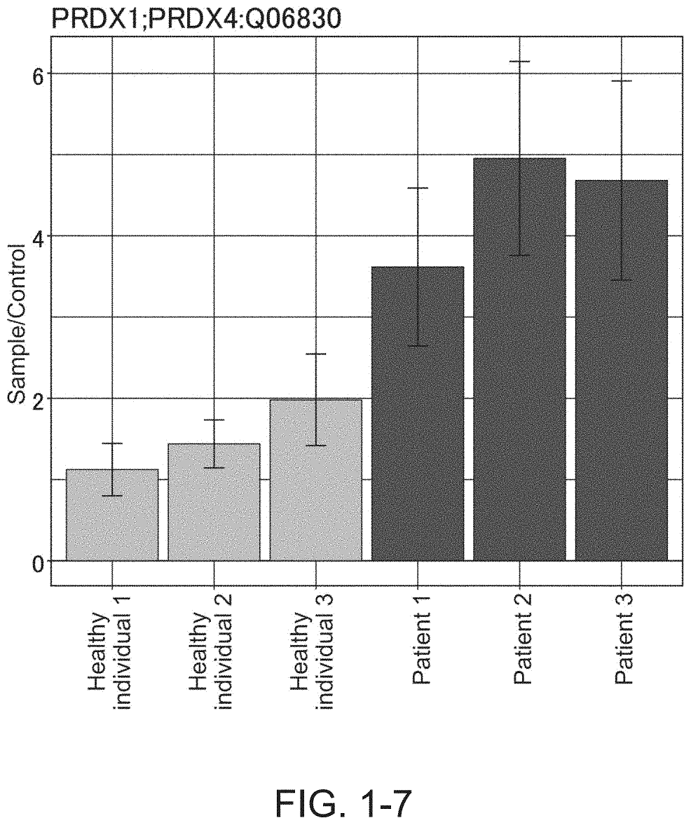

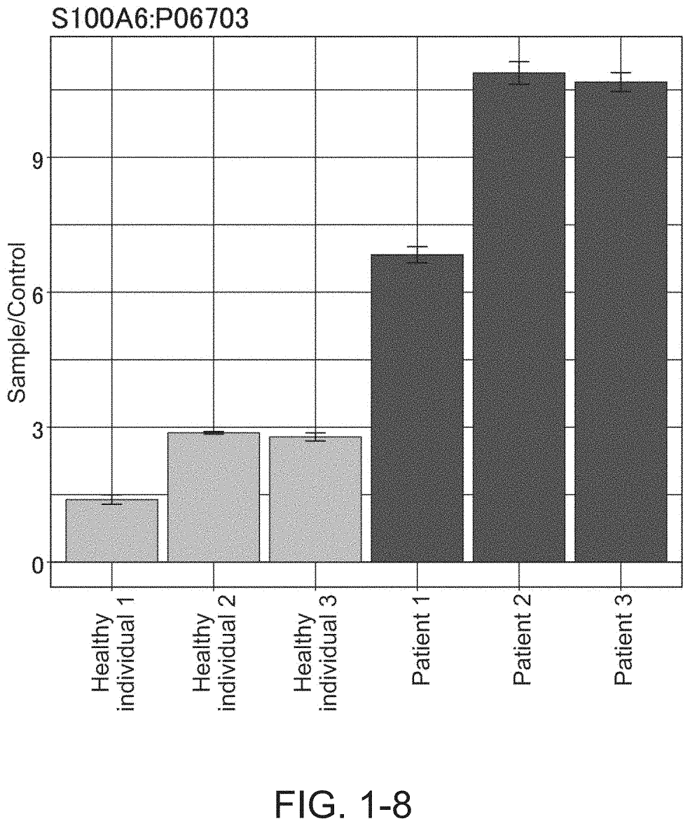

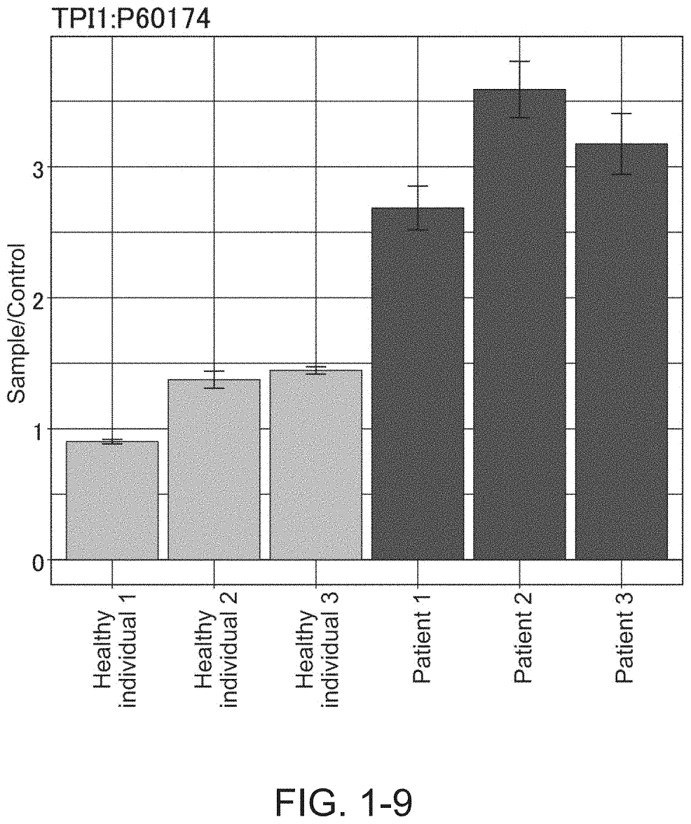

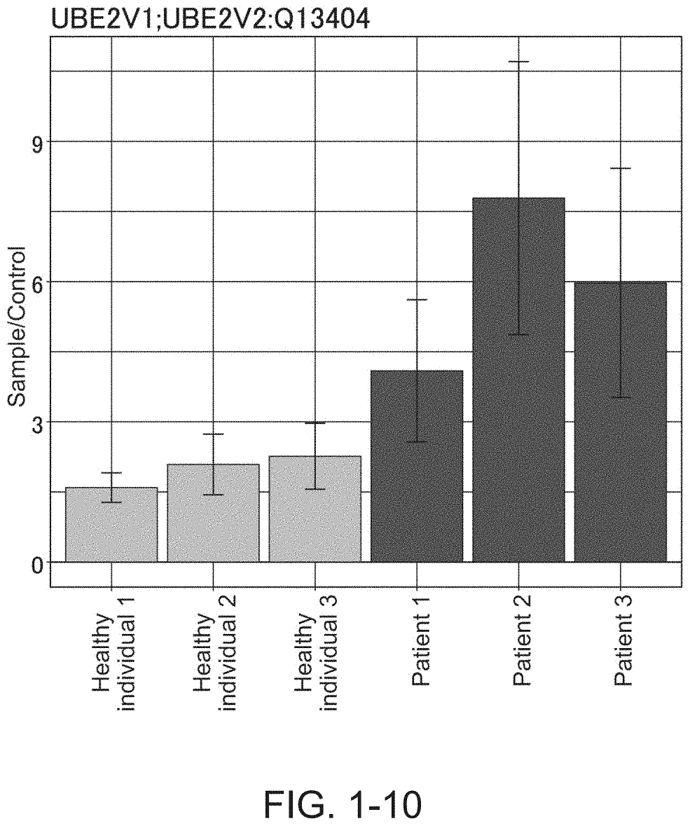

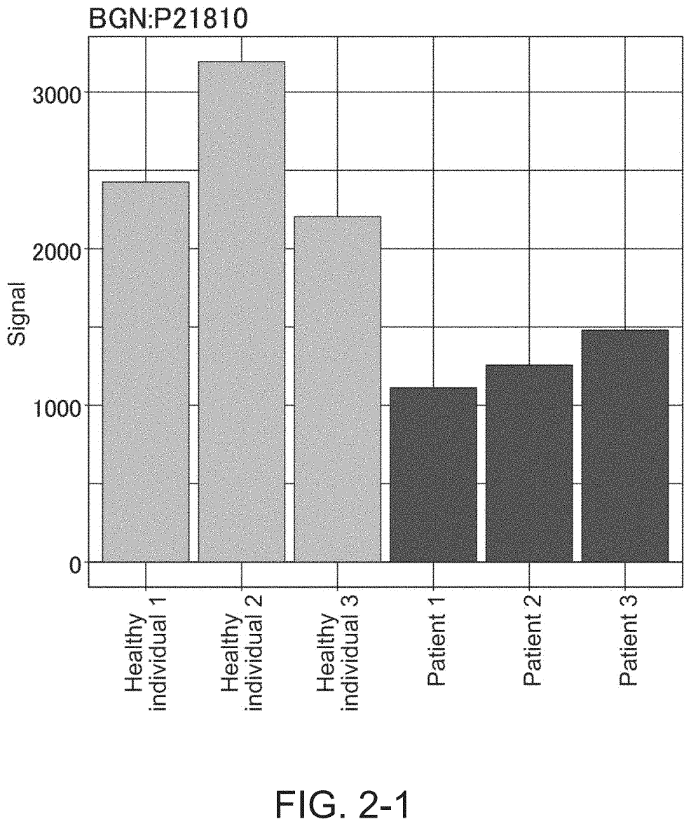

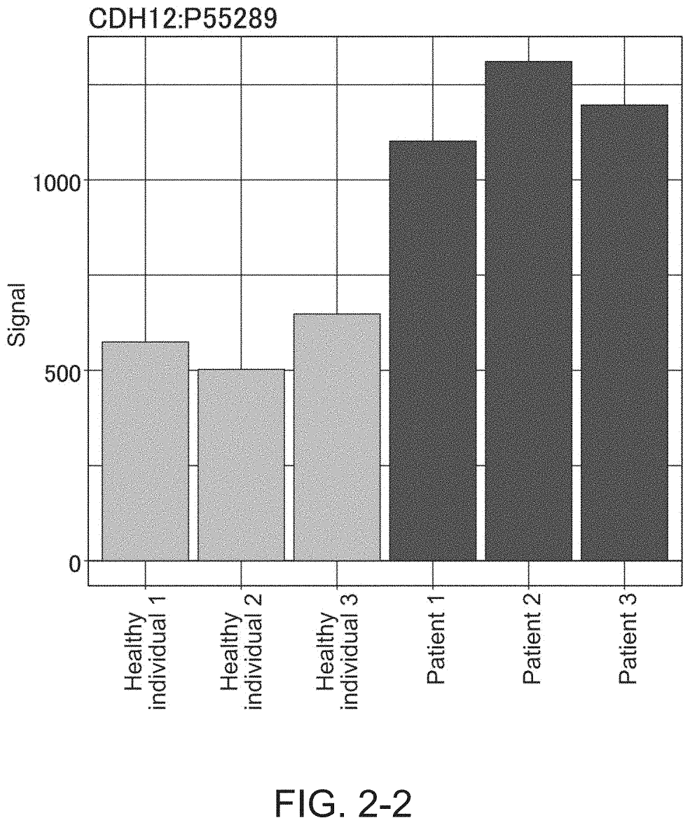

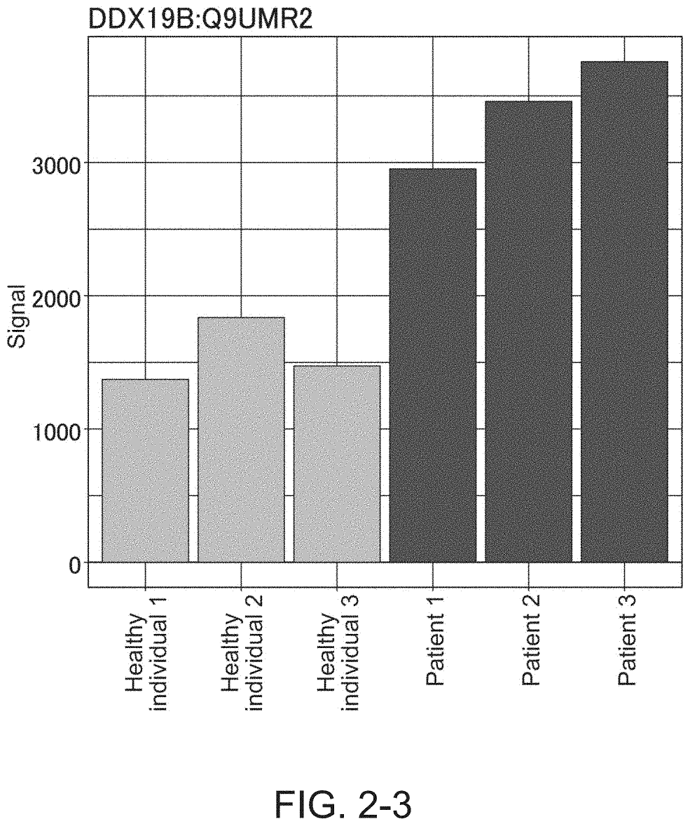

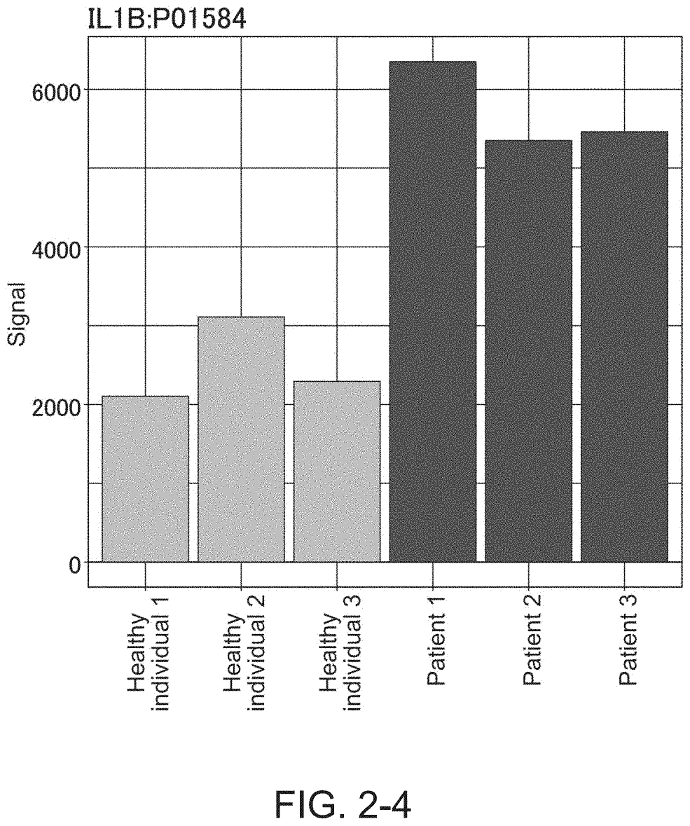

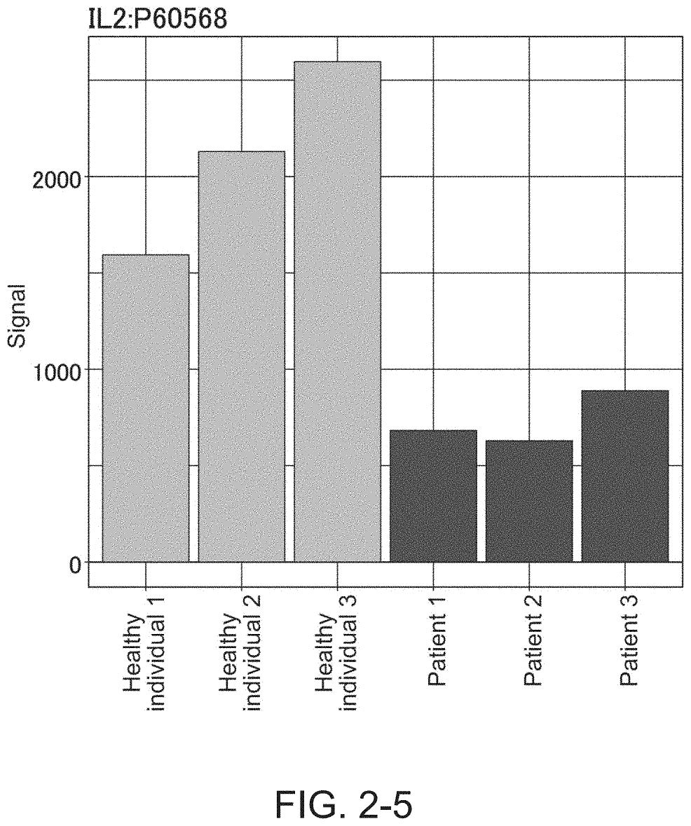

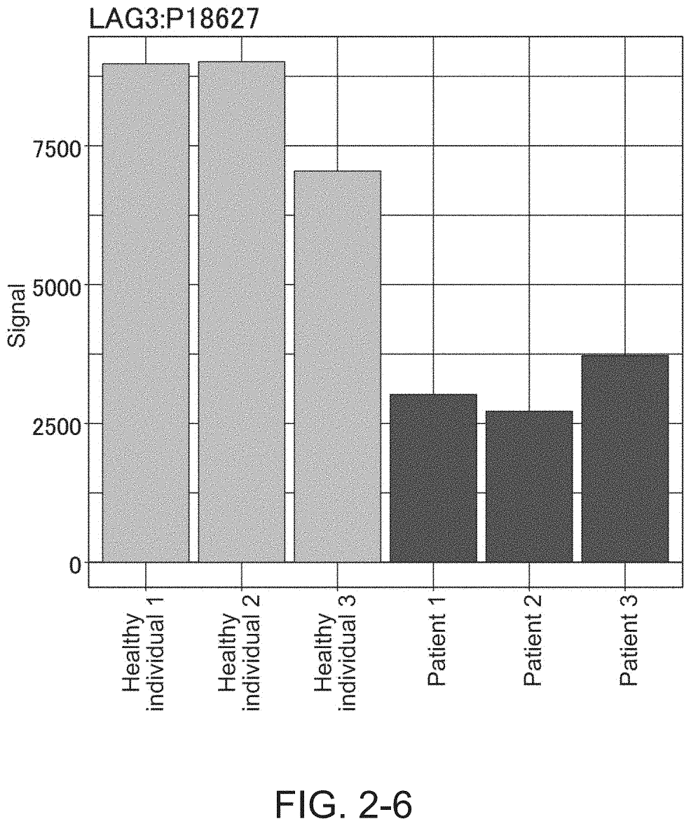

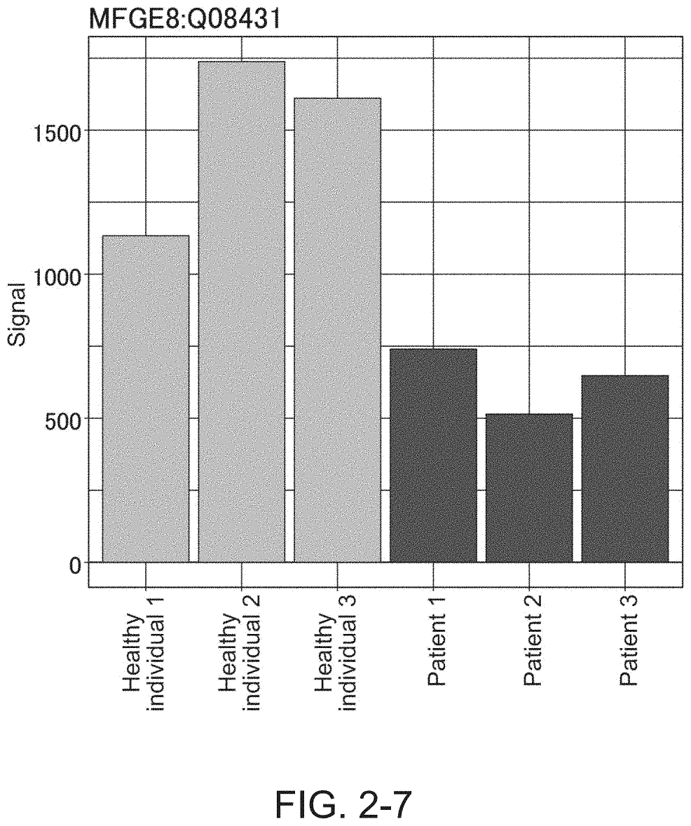

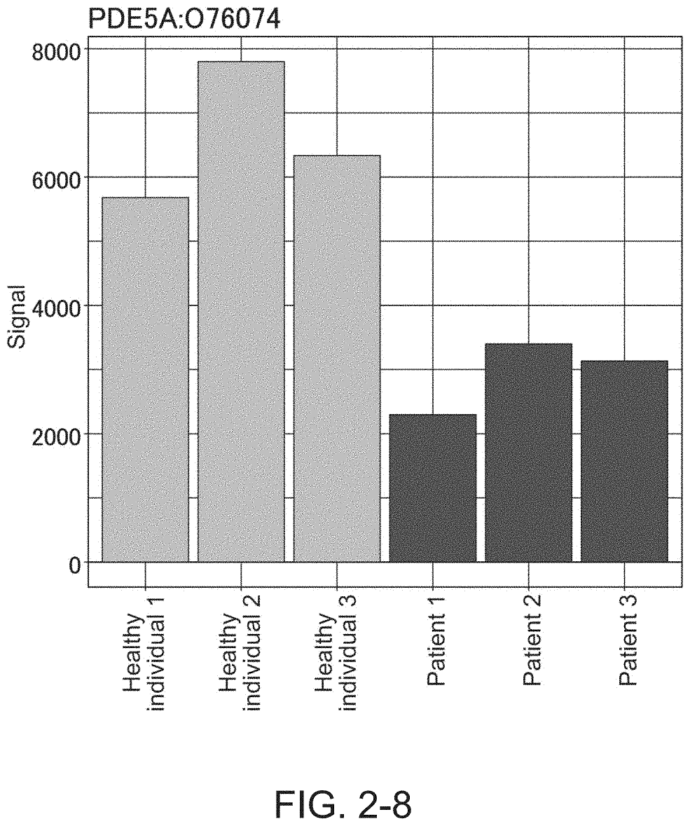

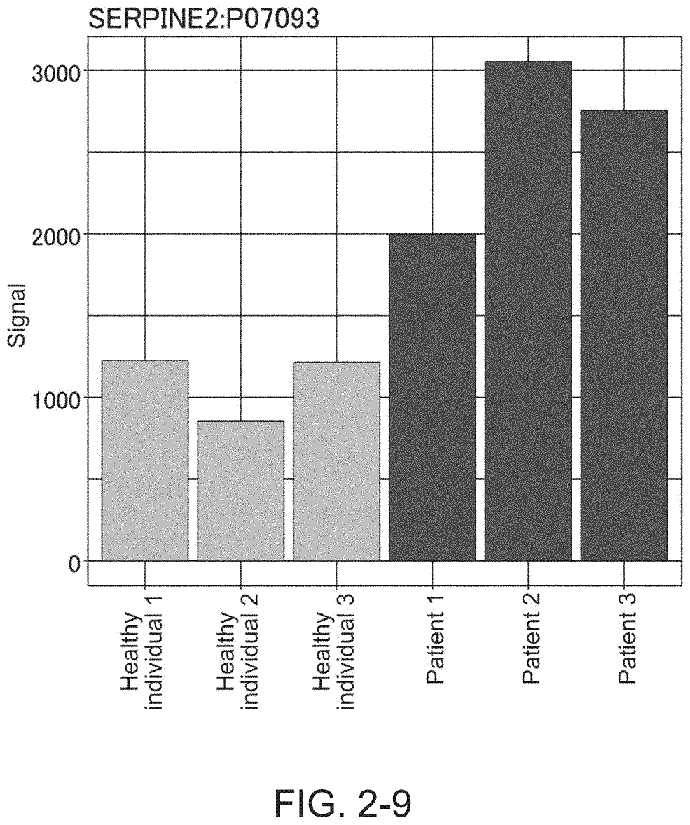

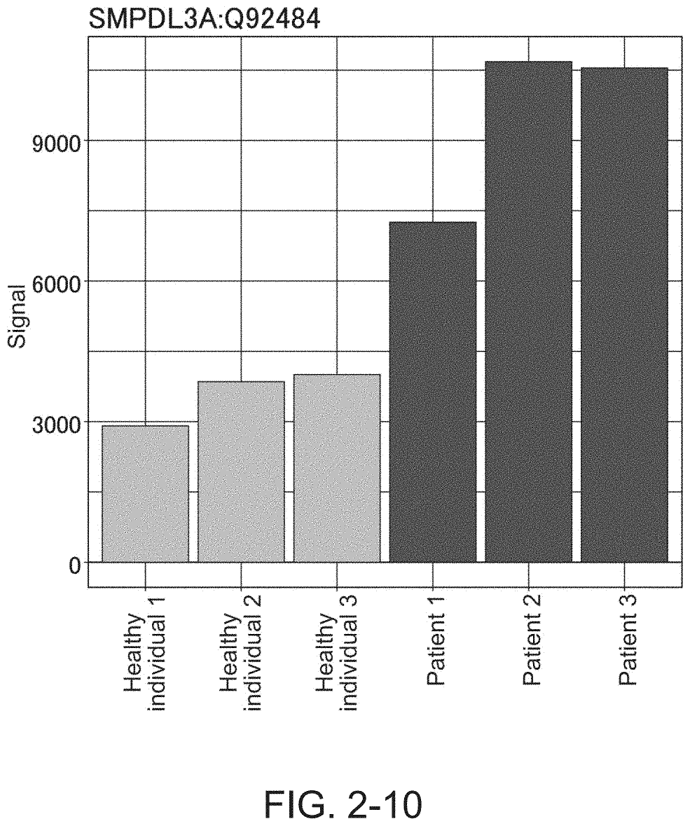

The selected proteins are promising as biomarkers for diagnosing endometriosis. FIGS. 1-1 to 1-10 show the graphs of relative expression levels of representative proteins among them.

### (Example 2) Search for Biomarkers by Aptamer-Binding Protein Analysis (SOMAscan®) Using Plasma of Healthy Human Individuals and Patients with Endometriosis

### (2-1) Obtainment of Human Plasma Samples

Plasma from healthy individuals and endometriosis patients was purchased from Proteogenex.

### (2-2) SOMAscan® Analysis of Plasma Proteins

Low-expressed proteins in plasma samples (three healthy individuals and three endometriosis patients) were analyzed by SOMAscan (registered trademark, Somalogic). Specifically, aptamers that each specifically binds to 1310 proteins were prepared, mixed with plasma, and allowed to bind to proteins in the plasma. The proteins bound to the aptamers were then biotinylated, and the aptamer-protein complexes were purified with streptavidin beads. Next, the aptamers were eluted and detected by a microarray, thereby the expression levels of the proteins bound to the aptamers in the plasma were analyzed.

### (2-3) Selection of Biomarker Candidates

The aptamer signal data of SOMAscan® were analyzed by statistical programming environment R and Microsoft Excel. According to the following criterion, 200 proteins for which altered expression was found in endometriosis patients were selected (Table 4A and Table 4B).

$$- \frac{\begin{matrix}The average value of the aptamer signal of the target \\ protein in three patients with endometriosis\end{matrix}}{\begin{matrix}The average value of the aptamer signal of the target \\ protein in three healthy individuals\end{matrix}} ≧ 2 or ≦ 0.5$$

In the above calculation, proteins with a maximum aptamer signal value of less than 1000 were considered to be below the detection limit and excluded from analysis.

TABLE 4A

| | | | | Aptamer signal: |
|---|---|---|---|---|
| | | Entrez | Entrez Gene | Average value of 3 patients/ |
| Protein name | UniProt ID | Gene ID | Symbol | Average value of 3 healthy individuals |
| Pulmonary surfactant-associated | P35247 | 6441 | SFTPD | 12.382 |
| protein D | | | | |
| Histone H1.2 | P16403 | 3006 | HIST1H1C | 11.014 |
| Sialic acid-binding Ig-like lectin 9 | Q9Y336 | 27180 | SIGLEC9 | 9.473 |
| Heterogeneous nuclear | P22626 | 3181 | HNRNPA2B1 | 7.516 |
| ribonucleoproteins A2/B1 | | | | |
| Hexokinase-2 | P52789 | 3099 | HK2 | 6.671 |
| High mobility group protein B1 | P09429 | 3146 | HMGB1 | 5.584 |
| Thrombospondin-1 | P07996 | 7057 | THBS1 | 5.365 |
| 3-hydroxy-3-methylglutaryl- | P04035 | 3156 | HMGCR | 4.718 |
| coenzyme A reductase | | | | |
| Platelet factor 4 | P02776 | 5196 | PF4 | 4.686 |
| Protein S100-A9 | P06702 | 6280 | S100A9 | 4.681 |
| 40S ribosomal protein S3a | P61247 | 6189 | RPS3A | 4.5 |
| Annexin A6 | P08133 | 309 | ANXA6 | 4.484 |
| Inosine-5′-monophosphate | P20839 | 3614 | IMPDH1 | 4.417 |
| dehydrogenase 1 | | | | |
| Tyrosine-protein kinase Fgr | P09769 | 2268 | FGR | 4.238 |
| Serine/threonine-protein kinase 17B | O94768 | 9262 | STK17B | 3.957 |
| Neutrophil-activating peptide 2 | P02775 | 5473 | PPBP | 3.826 |
| Estradiol 17-beta-dehydrogenase 1 | P14061 | 3292 | HSD17B1 | 3.787 |
| Connective tissue-activating | P02775 | 5473 | PPBP | 3.746 |
| peptide III | | | | |
| Prostaglandin G/H synthase 2 | P35354 | 5743 | PTGS2 | 3.686 |
| Amyloid beta A4 protein | P05067 | 351 | APP | 3.667 |
| GTP-binding nuclear protein Ran | P62826 | 5901 | RAN | 3.605 |
| NAD-dependent protein | Q8IXJ6 | 22933 | SIRT2 | 3.54 |
| deacetylase sirtuin-2 | | | | |
| Protein S100-A12 | P80511 | 6283 | S100A12 | 3.498 |
| Plasminogen activator inhibitor 1 | P05121 | 5054 | SERPINE1 | 3.367 |
| SUMO-conjugating enzyme UBC9 | P63279 | 7329 | UBE2I | 3.336 |
| Bactericidal permeability-increasing | P17213 | 671 | BPI | 3.321 |
| protein | | | | |
| 6-phosphogluconate | P52209 | 5226 | PGD | 3.204 |
| dehydrogenase, decarboxylating | | | | |
| Platelet-derived growth factor | P01127 | 5155 | PDGFB | 3.158 |
| subunit B | | | | |
| Tyrosine-protein phosphatase | P29350 | 5777 | PTPN6 | 3.117 |
| non-receptor type 6 | | | | |
| Metalloproteinase inhibitor 3 | P35625 | 7078 | TIMP3 | 3.113 |
| NudC domain-containing protein 3 | Q8IVD9 | 23386 | NUDCD3 | 3.112 |
| SPARC | P09486 | 6678 | SPARC | 3.046 |
| Protein 4.1 | P11171 | 2035 | EPB41 | 2.967 |
| Sex hormone-binding globulin | P04278 | 6462 | SHBG | 2.905 |
| Death-associated protein kinase 2 | Q9UIK4 | 23604 | DAPK2 | 2.834 |
| Hepatoma-derived growth | Q7Z4V5 | 84717 | HDGFRP2 | 2.777 |
| factor-related protein 2 | | | | |
| Proto-oncogene vav | P15498 | 7409 | VAV1 | 2.723 |
| Serine/threonine-protein kinase | P11309 | 5292 | PIM1 | 2.694 |
| pim-1 | | | | |
| Heterogeneous nuclear | Q99729 | 3182 | HNRNPAB | 2.685 |
| ribonucleoprotein A/B | | | | |
| Proliferation-associated | Q9UQ80 | 5036 | PA2G4 | 2.654 |
| protein 2G4 | | | | |
| Extracellular superoxide | P08294 | 6649 | SOD3 | 2.654 |
| dismutase [Cu-Zn] | | | | |
| Angiopoietin-1 | Q15389 | 284 | ANGPT1 | 2.643 |
| Acid sphingomyelinase-like | Q92484 | 10924 | SMPDL3A | 2.631 |
| phosphodiesterase 3a | | | | |
| Peroxiredoxin-6 | P30041 | 9588 | PRDX6 | 2.624 |
| AMP Kinase (alpha1beta1gamma1) | Q13131 | 5562 | PRKAA1 | 2.577 |
| | Q9Y478 | 5564 | PRKAB1 | |
| | P54619 | 5571 | PRKAG1 | |
| Serum amyloid A-1 protein | P0DJI8 | 6288 | SAA1 | 2.556 |
| Insulin-like growth factor-binding | P18065 | 3485 | IGFBP2 | 2.52 |
| protein 2 | | | | |
| Histone H2B type 2-E | Q16778 | 8349 | HIST2H2BE | 2.491 |
| Fibroblast growth factor 5 | P12034 | 2250 | FGF5 | 2.491 |
| Non-histone chromosomal protein | P05114 | 3150 | HMGN1 | 2.467 |
| HMG-14 | | | | |
| Calcium/calmodulin-dependent | Q13557 | 817 | CAMK2D | 2.464 |
| protein kinase type II subunit delta | | | | |
| Interstitial collagenase | P03956 | 4312 | MMP1 | 2.454 |
| ICOS ligand | O75144 | 23308 | ICOSLG | 2.444 |
| Calcium/calmodulin-dependent | Q13554 | 816 | CAMK2B | 2.425 |
| protein kinase type II subunit beta | | | | |
| Inosine-5′-monophosphate dehydrogenase 2 | P12268 | 3615 | IMPDH2 | 2.398 |
| Dickkopf-related protein 4 | Q9UBT3 | 27121 | DKK4 | 2.375 |
| Glia-derived nexin | P07093 | 5270 | SERPINE2 | 2.365 |
| 14-3-3 protein beta/alpha | P31946 | 7529 | YWHAB | 2.357 |
| Macrophage-capping protein | P40121 | 822 | CAPG | 2.352 |
| ADP-ribosyl cyclase/cyclic | P28907 | 952 | CD38 | 2.349 |
| ADP-ribose hydrolase 1 | | | | |
| SH2 domain-containing protein 1A | O60880 | 4068 | SH2D1A | 2.324 |
| Vacuolar protein sorting-associated | Q9NP79 | 51534 | VTA1 | 2.319 |
| protein VTA1 homolog | | | | |
| Interleukin-1 beta | P01584 | 3553 | IL1B | 2.312 |
| Chloride intracellular channel | O00299 | 1192 | CLIC1 | 2.306 |
| protein 1 | | | | |
| MAP kinase-activated protein | Q16644 | 7867 | MAPKAPK3 | 2.298 |
| kinase 3 | | | | |
| Mitogen-activated protein kinase 1 | P28482 | 5594 | MAPK1 | 2.295 |
| Choline/ethanolamine kinase | Q9Y259 | 1120 | CHKB | 2.293 |
| Creatine kinase B-type | P12277 | 1152 | CKB | 2.287 |
| DNA topoisomerase 1 | P11387 | 7150 | TOP1 | 2.286 |
| C-C motif chemokine 5 | P13501 | 6352 | CCL5 | 2.28 |
| C3a anaphylatoxin | P01024 | 718 | C3 | 2.276 |
| Copine-1 | Q99829 | 8904 | CPNE1 | 2.274 |
| Chymase | P23946 | 1215 | CMA1 | 2.27 |
| Mitogen-activated protein kinase | O43318 | 6885 | MAP3K7 | 2.27 |
| kinase kinase 7: TGF-beta-activated | Q15750 | 10454 | TAB1 | |
| kinase 1 and MAP3K7-binding | | | | |
| protein 1 fusion | | | | |
| Dickkopf-related protein 1 | O94907 | 22943 | DKK1 | 2.266 |
| Ephrin type-B receptor 4 | P54760 | 2050 | EPHB4 | 2.261 |
| 40S ribosomal protein S7 | P62081 | 6201 | RPS7 | 2.253 |
| Intercellular adhesion molecule 1 | P05362 | 3383 | ICAM1 | 2.248 |
| Ubiquitin + 1, truncated mutation | P62979 | 6233 | RPS27A | 2.222 |
| for UbB | | | | |
| Ubiquitin-conjugating enzyme E2 N | P61088 | 7334 | UBE2N | 2.219 |
| Moesin | P26038 | 4478 | MSN | 2.201 |
| Small ubiquitin-related modifier 3 | P55854 | 6613 | SUMO3 | 2.196 |
| G2/mitotic-specific cyclin-B1 | P14635 | 891 | CCNB1 | 2.19 |
| ATP-dependent RNA helicase | Q9UMR2 | 11269 | DDX19B | 2.178 |
| DDX19B | | | | |
| Tyrosine-protein kinase CSK | P41240 | 1445 | CSK | 2.17 |
| Alpha-1-antitrypsin | P01009 | 5265 | SERPINA1 | 2.162 |
| Adenylate kinase isoenzyme 1 | P00568 | 203 | AK1 | 2.145 |
| Mothers against decapentaplegic | P84022 | 4088 | SMAD3 | 2.137 |
| homolog 3 | | | | |
| Brain-derived neurotrophic factor | P23560 | 627 | BDNF | 2.136 |
| Atrial natriuretic factor | P01160 | 4878 | NPPA | 2.131 |
| Annexin A1 | P04083 | 301 | ANXA1 | 2.13 |
| Signal transducer and activator of | P42224 | 6772 | STAT1 | 2.12 |
| transcription 1-alpha/beta | | | | |
| Angiopoietin-related protein 4 | Q9BY76 | 51129 | ANGPTL4 | 2.104 |
| Cadherin-12 | P55289 | 1010 | CDH12 | 2.098 |
| Stress-induced-phosphoprotein 1 | P31948 | 10963 | STIP1 | 2.096 |
| Glycylpeptide | P30419 | 4836 | NMT1 | 2.091 |
| N-tetradecanoyltransferase 1 | | | | |
| Peroxiredoxin-1 | Q06830 | 5052 | PRDX1 | 2.077 |
| Oxidized low-density lipoprotein | P78380 | 4973 | OLR1 | 2.075 |
| receptor 1 | | | | |
| VPS10 domain-containing receptor | Q96PQ0 | 57537 | SORCS2 | 2.059 |
| SorCS2 | | | | |
| FACT complex subunit SSRP1 | Q08945 | 6749 | SSRP1 | 2.049 |
| Carbonic anhydrase 1 | P00915 | 759 | CA1 | 2.035 |
| Mitogen-activated protein kinase 11 | Q15759 | 5600 | MAPK11 | 2.034 |
| Triosephosphate isomerase | P60174 | 7167 | TPI1 | 2.029 |
| Neurexophilin-1 | P58417 | 30010 | NXPH1 | 2.018 |
| Platelet-derived growth factor | P04085 | 5154 | PDGFA | 2.017 |
| subunit A | | | | |
| Interleukin-22 receptor subunit | Q8N6P7 | 58985 | IL22RA1 | 2.004 |
| alpha-1 | | | | |

TABLE 4B

| | | | | Aptamer signal: |
|---|---|---|---|---|
| | | | | Average value of |
| | | | | 3 patients/Average |
| | | Entrez | Entrez | value of 3 healthy |
| Protein name | UniProt ID | Gene ID | Gene Symbol | individuals |
| Cystatin-SN | P01037 | 1469 | CST1 | 0.499 |
| Interleukin-22 | Q9GZX6 | 50616 | IL22 | 0.497 |
| Tyrosine-protein kinase Fyn | P06241 | 2534 | FYN | 0.497 |
| Biglycan | P21810 | 633 | BGN | 0.495 |
| Sonic hedgehog protein | Q15465 | 6469 | SHH | 0.492 |
| Cathepsin L2 | O60911 | 1515 | CTSV | 0.49 |
| Interferon alpha/beta receptor 1 | P17181 | 3454 | IFNAR1 | 0.486 |
| Ectodysplasin-A, secreted form | Q92838 | 1896 | EDA | 0.485 |
| Dynein light chain 1, cytoplasmic | P63167 | 8655 | DYNLL1 | 0.483 |
| Cytochrome c | P99999 | 54205 | CYCS | 0.482 |
| Lactoperoxidase | P22079 | 4025 | LPO | 0.48 |
| Tumor necrosis factor receptor | Q969Z4 | 84957 | RELT | 0.48 |
| superfamily member 19L | | | | |
| Growth factor receptor-bound protein 2 | P62993 | 2885 | GRB2 | 0.467 |
| Ectonucleotide pyrophosphatase/ | Q6UWV6 | 339221 | ENPP7 | 0.466 |
| phosphodiesterase family member 7 | | | | |
| Glucagon | P01275 | 2641 | GCG | 0.466 |
| Eukaryotic translation initiation factor 4 | P78344 | 1982 | EIF4G2 | 0.465 |
| gamma 2 | | | | |
| C-X-C motif chemokine 10 | P02778 | 3627 | CXCL10 | 0.457 |
| Platelet-derived growth factor receptor | P09619 | 5159 | PDGFRB | 0.453 |
| beta | | | | |
| von Willebrand factor | P04275 | 7450 | VWF | 0.452 |
| Iduronate 2-sulfatase | P22304 | 3423 | IDS | 0.443 |
| cGMP-specific 3′,5′-cyclic | O76074 | 8654 | PDE5A | 0.443 |
| phosphodiesterase | | | | |
| Small glutamine-rich tetratricopeptide | O43765 | 6449 | SGTA | 0.441 |
| repeat-containing protein alpha | | | | |
| Interleukin-25 | Q9H293 | 64806 | IL25 | 0.433 |
| MHC class I polypeptide-related | Q29980 | 4277 | MICB | 0.433 |
| sequence B | | | | |
| C-C motif chemokine 7 | P80098 | 6354 | CCL7 | 0.429 |
| Lactadherin | Q08431 | 4240 | MFGE8 | 0.427 |
| Macrophage metalloelastase | P39900 | 4321 | MMP12 | 0.425 |
| Ubiquitin-like protein ISG15 | P05161 | 9636 | ISG15 | 0.423 |
| Fatty acid-binding protein, liver | P07148 | 2168 | FABP1 | 0.418 |
| Properdin | P27918 | 5199 | CFP | 0.417 |
| Stromelysin-2 | P09238 | 4319 | MMP10 | 0.415 |
| Protein FAM107B | Q9H098 | 83641 | FAM107B | 0.413 |
| Myosin-binding protein C, slow-type | Q00872 | 4604 | MYBPC1 | 0.413 |
| Serine/threonine-protein kinase Chk2 | O96017 | 11200 | CHEK2 | 0.41 |
| Xaa-Pro aminopeptidase 1 | Q9NQW7 | 7511 | XPNPEP1 | 0.395 |
| NT-3 growth factor receptor | Q16288 | 4916 | NTRK3 | 0.394 |
| Interleukin-5 receptor subunit alpha | Q01344 | 3568 | IL5RA | 0.392 |
| Ephrin type-A receptor 2 | P29317 | 1969 | EPHA2 | 0.391 |
| Peptidyl-prolyl cis-trans isomerase F, | P30405 | 10105 | PPIF | 0.387 |
| mitochondrial | | | | |
| B-cell lymphoma 6 protein | P41182 | 604 | BCL6 | 0.384 |
| Cell adhesion molecule 1 | Q9BY67 | 23705 | CADM1 | 0.384 |
| Kinesin-like protein KIF23 | Q02241 | 9493 | KIF23 | 0.383 |
| Netrin receptor UNC5D | Q6UXZ4 | 137970 | UNC5D | 0.381 |
| Lymphocyte activation gene 3 protein | P18627 | 3902 | LAG3 | 0.377 |
| Serine protease HTRA2, mitochondrial | O43464 | 27429 | HTRA2 | 0.376 |
| Interleukin-22 receptor subunit alpha-2 | Q969J5 | 116379 | IL22RA2 | 0.375 |
| Caspase-3 | P42574 | 836 | CASP3 | 0.374 |
| Platelet-derived growth factor receptor | P16234 | 5156 | PDGFRA | 0.372 |
| alpha | | | | |
| Interleukin-20 | Q9NYY1 | 50604 | IL20 | 0.368 |
| Ferritin | P02794 P02792 | 2495 2512 | FTH1 FTL | 0.367 |
| Ephrin-A5 | P52803 | 1946 | EFNA5 | 0.361 |
| Amphoterin-induced protein 2 | Q86SJ2 | 347902 | AMIGO2 | 0.353 |
| Interleukin-2 | P60568 | 3558 | IL2 | 0.351 |
| Phospholipase A2 | P04054 | 5319 | PLA2G1B | 0.346 |
| Protein kinase C alpha type | P17252 | 5578 | PRKCA | 0.345 |
| Tumor necrosis factor ligand | P32971 | 944 | TNFSF8 | 0.343 |
| superfamily member 8 | | | | |
| Endoglin | P17813 | 2022 | ENG | 0.342 |
| Gamma-enolase | P09104 | 2026 | ENO2 | 0.332 |
| C-X-C motif chemokine 9 | Q07325 | 4283 | CXCL9 | 0.329 |
| beta-nerve growth factor | P01138 | 4803 | NGF | 0.327 |
| Hepcidin | P81172 | 57817 | HAMP | 0.323 |
| Inorganic pyrophosphatase | Q15181 | 5464 | PPA1 | 0.319 |
| Kallikrein-8 | O60259 | 11202 | KLK8 | 0.314 |
| Semaphorin-6B | Q9H3T3 | 10501 | SEMA6B | 0.312 |
| Adapter molecule crk | P46108 | 1398 | CRK | 0.307 |
| Phosphoglycerate mutase 1 | P18669 | 5223 | PGAM1 | 0.298 |
| Proprotein convertase subtilisin/ | Q16549 | 9159 | PCSK7 | 0.298 |
| kexin type 7 | | | | |
| Pancreatic hormone | P01298 | 5539 | PPY | 0.297 |
| Interleukin-1 receptor type 1 | P14778 | 3554 | IL1R1 | 0.296 |
| C-X-C motif chemokine 11 | O14625 | 6373 | CXCL11 | 0.293 |
| Secreted frizzled-related protein 1 | Q8N474 | 6422 | SFRP1 | 0.288 |
| Inhibin beta A chain | P08476 | 3624 | INHBA | 0.284 |
| Immunoglobulin D | P01880 | 3495 50802 3535 | IGHD IGK@ | 0.277 |
| | | | IGL@ | |
| Complement C3b | P01024 | 718 | C3 | 0.272 |
| Teratocarcinoma-derived growth | P13385 | 6997 | TDGF1 | 0.27 |
| factor 1 | | | | |
| Brother of CDO | Q9BWV1 | 91653 | BOC | 0.269 |
| CD109 antigen | Q6YHK3 | 135228 | CD109 | 0.26 |
| Aminoacylase-1 | Q03154 | 95 | ACY1 | 0.259 |
| 60 kDa heat shock protein, | P10809 | 3329 | HSPD1 | 0.255 |
| mitochondrial | | | | |
| Importin subunit beta-1 | Q14974 | 3837 | KPNB1 | 0.253 |
| C-reactive protein | P02741 | 1401 | CRP | 0.251 |
| Ephrin type-A receptor 1 | P21709 | 2041 | EPHA1 | 0.242 |
| Semaphorin-6A | Q9H2E6 | 57556 | SEMA6A | 0.24 |
| SLIT and NTRK-like protein 5 | O94991 | 26050 | SLITRK5 | 0.233 |
| Leucine-rich repeat transmembrane | Q9NZU0 | 23767 | FLRT3 | 0.217 |
| protein FLRT3 | | | | |
| dCTP pyrophosphatase 1 | Q9H773 | 79077 | DCTPP1 | 0.21 |
| Osteopontin | P10451 | 6696 | SPP1 | 0.206 |
| Formimidoyltransferase-cyclodeaminase | O95954 | 10841 | FTCD | 0.191 |
| Ephrin-B2 | P52799 | 1948 | EFNB2 | 0.175 |
| T-lymphocyte surface antigen Ly-9 | Q9HBG7 | 4063 | LY9 | 0.17 |
| Sialic acid-binding Ig-like lectin 14 | Q08ET2 | 100049587 | SIGLEC14 | 0.157 |
| N-acylethanolamine-hydrolyzing acid | Q02083 | 27163 | NAAA | 0.146 |
| amidase | | | | |
| Endoplasmic reticulum aminopeptidase 1 | Q9NZ08 | 51752 | ERAP1 | 0.118 |
| Low affinity immunoglobulin gamma Fc | P12318 | 2212 | FCGR2A | 0.053 |
| region receptor II-a | | | | |

The selected proteins are promising as biomarkers for diagnosing endometriosis. FIGS. 2-1 to 2-10 show the graphs of expression levels of representative proteins among them.

### (Example 3) Search for Biomarkers by Extracellular Matrix Degradation Product Analysis Using Plasma of Healthy Human Individuals and Patients with Endometriosis

### (3-1) Obtainment of Human Plasma Samples

Plasma from healthy individuals and endometriosis patients was purchased from Proteogenex.

### (3-2) Extracellular Matrix Measurement of Plasma Proteins

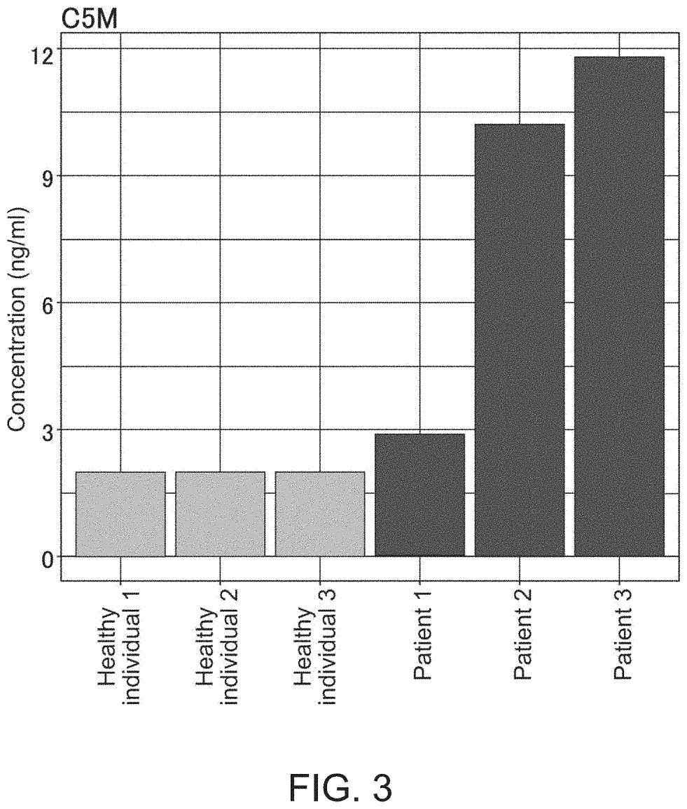

Plasma samples (three healthy individuals and three endometriosis patients) were analyzed by the ELISA system with an antibody specific to the cleavage site of the extracellular matrix, and the total amount of extracellular matrix and the degradation/synthesis state of extracellular matrix in the samples were analyzed (Nordic Bioscience). Specifically, with antibodies against the cleavage site contained in degradation products when type III, IV, V, or VI collagen, decorin, or nidogen was degraded, the expression level of each degradation product was analyzed. The result showed that the type V collagen MMP degradation product (C5M) was remarkably increased in two of the three patients (the C5M concentration was 10.2 ng/ml and 11.8 ng/ml in the plasma of the two patients with increased C5M), while the plasma C5M concentration was below the detection limit in all healthy individuals. Thus, it was suggested that C5M is promising as a biomarker for endometriosis. FIG. 3 shows the graph of the plasma concentration of the type V collagen MMP degradation product (C5M).

### (Example 4) Search for Biomarkers by Luminex xMAP™ Technology Using Plasma of Healthy Human Individuals and Patients with Endometriosis

### (4-1) Obtainment of Human Plasma Samples

Plasma of healthy individuals (five plasma samples at secretory stage and five plasma samples at proliferative stage) was purchased from Proteogenex. As plasma of endometriosis patients, a total of 111 plasma samples were obtained from 37 endometriosis patients, each before surgery, at 3 days after surgery, and at 1 month after surgery.

### (4-2) Analysis of Plasma Proteins by Luminex xMAP™ Technology

Proteins in plasma samples were analyzed by Luminex xMAP™ (a trademark of Luminex). The Luminex xMAP™ technology is a technology of staining macrobeads with two fluorescent dyes combined at various concentrations and immobilizing onto each bead a substance that binds to each analysis target, whereby multiple items can be simultaneously analyzed with a small amount of samples using the content of the fluorescent dyes as an identification code.

In this Example, 152 proteins were selected and analyzed using protein measurement panels. Specifically, an antibody that binds to a target protein was immobilized on beads for each panel, a plasma sample was reacted with the antibody on the beads, a reporter antibody (labeled with a fluorescent dye) against the target protein was further reacted, and the expression level of the target protein was analyzed by measuring the fluorescence intensity with two types of lasers by flow cytometry technology (using the Luminex100 device).

### (4-3) Selection of Biomarker Candidates

The plasma concentrations of the obtained various proteins were analyzed by Microsoft Excel. According to the following criterion, 24 proteins for which altered expression was found in endometriosis patients were selected (Table 7A and Table 7B).

$$\frac{\begin{matrix}The average value of the plasma concentration of the protein \\ in 37 patients with endometriosis before surgery\end{matrix}}{\begin{matrix}\begin{matrix}The average value of the plasma concentration of the protein \\ in 10 samples from 5 healthy individuals at \end{matrix} \\ secretory and proliferative stages\end{matrix}} ≧ 2 or ≦ 0.5$$

Of the 24 proteins selected, 12 proteins showed the highest value in endometriosis patients at the time of surgery and a decreased value one month after surgery, and also showed the lowest value in healthy individuals.

TABLE 7A

| | | | | Patients with endometriosis |
|---|---|---|---|---|
| | | | | (before surgery) > Patients |
| | | | | with endometriosis (1 |
| | | | Abundance | month after surgery) > |
| UniProt ID | Protein name | Gene name | ratio | Healthy individuals |
| Q15389 | Angiopoietin-1 (ANG-1) | ANGPT1 | 2.96 | ∘ |
| P23560 | Brain-Derived Neurotrophic Factor (BDNF) | BDNF | 4.03 | ∘ |
| O94907 | Dickkopf-related protein 1 (DKK-1) | DKK1 | 2.11 | ∘ |
| P80511 | S100-A12 | S100A12 | 4.67 | ∘ |
| P42830 | Epithelial-Derived Neutrophil-Activating | CXCL5 | 2.52 | ∘ |
| | Protein 78 (ENA-78) | | | |
| P02794 | Ferritin (FRTN) | FTH1 | 2.99 | |
| P09341 | Growth-Regulated alpha protein (GRO-alpha) | CXCL1 | 2.23 | ∘ |
| P01137 | Latency-Associated Peptide of Transforming | TGFB1 | 2.55 | ∘ |
| | Growth Factor beta 1 (LAPTGF-b1) | | | |
| Q8WXL0 | Luteinizing Hormone (LH) | LHB | 2.26 | ∘ |
| G3CBL7 | MHC class I chain-related protein A (MICA) | MICA | 2.29 | |
| P02775 | Platelet basic protein | PPBP | 2.92 | ∘ |
| P01127 | Platelet-Derived Growth Factor BB (PDGF-BB) | PDGFB | 4.25 | ∘ |
| P01236 | Prolactin (PRL) | PRL | 3.76 | ∘ |
| P0DJI8; P0DJI9-2 | Serum amyloid A-1 protein; Amyloid protein | SAA1; SAA2 | 3.01 | |
| | A; Serum amyloid protein A(2-104); Serum | | | |
| | amyloid protein A(3-104); Serum amyloid | | | |
| | protein A(2-103); Serum amyloid protein | | | |
| | A(2-102); Serum amyloid protein A(4- | | | |
| | 101); Serum amyloid A-2 protein | | | |
| P13501 | T-Cell-Specific Protein RANTES (RANTES) | CCL5 | 2.18 | ∘ |

TABLE 7B

| UniProt ID | Protein name | Gene name | Abundance ratio |
|---|---|---|---|
| P10645 | Chromogranin-A (CgA) | CHGA | 0.38 |
| Q9GZV9 | Fibroblast growth factor 23 (FGF-23) | FGF23 | 0.22 |
| P01266 | Thyroglobulin (TG) | TG | 0.47 |

### (Example 5) Search for Biomarkers by LC-MS Analysis Using Plasma of Healthy Human Individuals and Patients with Endometriosis

### (5-1) Obtainment of Human Plasma Samples

Plasma of healthy individuals (five plasma samples at secretory stage and five plasma samples at proliferative stage) was purchased from Proteogenex. As plasma of endometriosis patients, a total of 125 plasma samples were obtained from 39 endometriosis patients, each before surgery, at 3 days after surgery, and at 1 month after surgery.

### (5-2) LC-MS Analysis of Plasma Proteins

With High Select™ Top14 Abundant Protein Depletion Mini Spin Columns (Thermo), highly abundant proteins in each plasma sample were bound to the column and removed by the method recommended by the manufacturer. The proteins in the flow-through fraction that did not bind to the column were precipitated by the methanol/chloroform method and dissolved in 8 M urea/400 mM ammonium bicarbonate solution. The dissolved proteins were reduced and alkylated, and then digested into peptides with lysyl endopeptidase and trypsin. The peptide solution was demineralized with Monospin C18 (GL Science) by the method recommended by the manufacturer.

The peptides were labeled with TMT10plex by the method recommended by the manufacturer. With AssayMAP reversed phase (RP-S) cartridges (Agilent technologies), the labeled peptides were fractionated by the High pH Reversed-Phase method with triethylamine Each fraction was analyzed by the LC-MS analysis system in which Orbitrap Fusion Lumos (ThermoFisher scientific) was coupled to the nano-LC system (EASY-nLC™ 1200 system, ThermoFisher Scientific). The analysis samples were peptides obtained from the plasma of each of five healthy individuals (each at secretory stage and proliferative stage; a total of 10 samples) and 39 endometriosis patients (before surgery, at 3 days after surgery, and at 1 month after surgery; a total of 125 samples). The sample prepared by mixing an equal amount of seven samples from healthy individuals and seven samples from endometriosis patients, which were purchased for correction between measurements, was used as a control in each analysis, and 10 samples were used for each analysis. Each sample was analyzed three times, and they were integrated during analysis.

The proteins in the samples were identified from the raw data of the LC-MS analysis by Thermo Scientific™ Proteome Discoverer™, and their relative expression levels were analyzed. The database search for protein identification was performed with the following parameters.

Taxonomy: uniprot human
Static modifications: TMT6plex/+229.163 Da (Any N-Terminus), Carbamidomethyl/+57.021 Da (C, C-Terminus), TMT6plex /+229.163 Da (K, C-Terminus)

### Dynamic Modifications: Oxidation/+15.995 Da (M), Acetyl/+42.011 Da (N-Terminus)

### FDR: Target FDR (Strict)/0.01, Target FDR (Relaxed)/0.05

The relative expression levels of the target proteins in the plasma samples from endometriosis patients before surgery relative to the target proteins in the plasma samples from healthy individuals were calculated by the analysis software (Proteome Discoverer).

### (5-3) Selection of Biomarker Candidate Molecules

The LC-MS result data were analyzed by Proteome Discoverer. From the relative expression level of each protein in the samples (10 samples from five healthy individuals at secretory stage and proliferative stage and samples from 39 endometriosis patients before surgery), 27 proteins for which altered expression was found in endometriosis patients were selected according to the following criteria (Table 8A and Table 8B).
For the abundance ratio, P-Value≤0.5 in ANOVA and tukey HSD post hoc; and
Abundance ratios≤0.6250 or ≥1.6000

$$Abundance ratio = \frac{\begin{matrix}The median value of the relative expression level of the protein \\ in 39 patients with edometriosis before surgery\end{matrix}}{\begin{matrix}The median value of the relative expression level of the protein \\ in 10 samples from 5 healthy individuals at \\ secretory and proliferative stages\end{matrix}}$$

Of the 27 proteins selected, 16 proteins showed the highest value in endometriosis patients at the time of surgery and a decreased value one month after surgery, and also showed the lowest value in healthy individuals.

TABLE 8A

| | | | | Patients with endometriosis |
|---|---|---|---|---|
| | | | | (before surgery) > Patients |
| | | | | with endometriosis (1 |
| | | | Abundance | month after surgery) > |
| UniProt ID | Protein name | Gene name | ratio | Healthy individuals |
| P07996 | Thrombospondin-1 OS = Homo sapiens | THBS1 | 3.149 | ∘ |
| | OX = 9606 GN = THBS1 PE = 1 SV = 2 | | | |
| P69905 | Hemoglobin subunit alpha OS = Homo sapiens | HBA1 | 1.717 | ∘ |
| | OX = 9606 GN = HBA1 PE = 1 SV = 2 | | | |
| P32119 | Peroxiredoxin-2 OS = Homo sapiens | PRDX2 | 1.772 | ∘ |
| | OX = 9606 GN = PRDX2 PE = 1 SV = 5 | | | |
| P00918 | Carbonic anhydrase 2 OS = Homo sapiens | CA2 | 1.821 | ∘ |
| | OX = 9606 GN = CA2 PE = 1 SV = 2 | | | |
| P30043 | Flavin reductase (NADPH) OS = Homo sapiens | BLVRB | 1.787 | ∘ |
| | OX = 9606 GN = BLVRB PE = 1 SV = 3 | | | |
| O76011 | Keratin, type I cuticular Ha4 OS = Homo sapiens | KRT34 | 2.091 | ∘ |
| | OX = 9606 GN = KRT34 PE = 1 SV = 2 | | | |
| P13716 | Delta-aminolevulinic acid dehydratase OS = | ALAD | 1.867 | ∘ |
| | Homo sapiens OX = 9606 GN = ALAD PE = 1 SV = 1 | | | |
| P00568 | Adenylate kinase isoenzyme 1 OS = Homo sapiens | AK1 | 1.786 | ∘ |
| | OX = 9606 GN = AK1 PE = 1 SV = 3 | | | |
| P07738 | Bisphosphoglycerate mutase OS = Homo sapiens | BPGM | 1.735 | ∘ |
| | OX = 9606 GN = BPGM PE = 1 SV = 2 | | | |
| P02775 | Platelet basic protein OS = Homo sapiens | PPBP | 3.628 | ∘ |
| | OX = 9606 GN = PPBP PE = 1 SV = 3 | | | |
| P22392 | Nucleoside diphosphate kinase B OS = | NME2 | 1.779 | ∘ |
| | Homo sapiens OX = 9606 GN = NME2 PE = 1 SV = 1 | | | |
| P10124 | Serglycin OS = Homo sapiens | SRGN | 3.181 | ∘ |
| | OX = 9606 GN = SRGN PE = 1 SV = 3 | | | |
| A0A0C4DH67 | Immunoglobulin kappa variable 1-8 OS = | IGKV1-8 | 1.615 | x |
| | Homo sapiens OX = 9606 GN = IGKV1-8 PE = 3 SV = 1 | | | |
| P63241 | Eukaryotic translation initiation factor 5A-1 OS = | EIF5A | 1.874 | ∘ |
| | Homo sapiens OX = 9606 GN = EIF5A PE = 1 SV = 2 | | | |
| P02776 | Platelet factor 4 OS = Homo sapiens | PF4 | 3.438 | ∘ |
| | OX = 9606 GN = PF4 PE = 1 SV = 2 | | | |
| A0A075B6S2 | Immunoglobulin kappa variable 2D-29 OS = | IGKV2D-29 | 2.174 | x |
| | Homo sapiens OX = 9606 GN = IGKV2D-29 PE = 3 SV = 1 | | | |
| Q9UKU6 | Thyrotropin-releasing hormone-degrading ectoenzyme | TRHDE | 1.879 | ∘ |
| | OS = Homo sapiens OX = 9606 GN = TRHDE PE = 2 SV = 1 | | | |
| Q8WUM4 | Programmed cell death 6-interacting protein OS = | PDCD6IP | 1.644 | x |
| | Homo sapiens OX = 9606 GN = PDCD6IP PE = 1 SV = 1 | | | |
| P00441 | Superoxide dismutase [Cu—Zn] OS = Homo sapiens | SOD1 | 2.01 | ∘ |
| | OX = 9606 GN = SOD1 PE = 1 SV = 2 | | | |

TABLE 8B

| UniProt ID | Protein name | Gene name | Abundance ratio |
|---|---|---|---|
| P36980 | Complement factor H-related protein 2 OS = Homo sapiens | CFHR2 | 0.443 |
| | OX = 9606 GN = CFHR2 PE = 1 SV = 1 | | |
| P0D0X3 | Immunoglobulin delta heavy chain OS = Homo sapiens | N/A | 0.518 |
| | OX = 9606 PE = 1 SV = 1 | | |
| P01880 | Immunoglobulin heavy constant delta OS = Homo sapiens | IGHD | 0.456 |
| | OX = 9606 GN = IGHD PE = 1 SV = 3 | | |
| Q8N6C8 | Leukocyte immunoglobulin-like receptor subfamily A member | LILRA3 | 0.318 |
| | 3 OS = Homo sapiens OX = 9606 GN = LILRA3 PE = 1 SV = 3 | | |
| P47929 | Galectin-7 OS = Homo sapiens | LGALS7 | 0.533 |
| | OX = 9606 GN = LGALS7 PE = 1 SV = 2 | | |
| O75339 | Cartilage intermediate layer protein 1 OS = Homo sapiens | CILP | 0.614 |
| | OX = 9606 GN = CILP PE = 1 SV = 4 | | |
| O15389 | Sialic acid-binding Ig-like lectin 5 OS = Homo sapiens | SIGLEC5 | 0.546 |
| | OX = 9606 GN = SIGLEC5 PE = 1 SV = 1 | | |
| P35247 | Pulmonary surfactant-associated protein D OS = | SFTPD | 0.605 |
| | Homo sapiens OX = 9606 GN = SFTPD PE = 1 SV = 3 | | |

### INDUSTRIAL APPLICABILITY

The present disclosure proved that by measuring the concentration of a type V collagen MMP degradation product or the abundance of at least one marker selected from the group consisting of the markers shown in Table 1A, the markers shown in Table 2A, the markers shown in Table 1B, the markers shown in Table 2B, the markers shown in Table 5A, and the markers shown in Table 6A, the markers shown in Table 5B, and the markers shown in Table 6B in a sample obtained from a subject, whether or not the subject is affected by endometriosis can be diagnosed. The invention of the present disclosure allows for the diagnosis of endometriosis by non-invasive means and is highly useful in the diagnosis and treatment of the disease.

1. A method of diagnosing endometriosis, comprising measuring the abundance of at least one marker selected from the group consisting of a type V collagen MMP degradation product, the markers shown in Group 1A, the markers shown in Group 2A, the markers shown in Group 1B, the markers shown in Group 2B, the markers shown in Group 3A, the markers shown in Group 4A, the markers shown in Group 3B, and the markers shown in Group 4B in a sample obtained from a subject.
Group 1A

| UniProt ID | Protein name | Gene name |
|---|---|---|
| P02042 | Hemoglobin subunit delta | HBD |
| P68871 | Hemoglobin subunit beta; LVV-hemorphin-7; | HBB |
| | Spinorphin | |
| P55072 | Transitional endoplasmic reticulum ATPase | VCP |
| P50395; P50395-2 | Rab GDP dissociation inhibitor beta | GDI2 |
| O43852-9; O43852-5; O43852-2; | Calumenin | CALU |
| O43852; O43852-4; O43852-3; | | |
| O43852-12; O43852-13; O43852-14; | | |
| O43852-7; O43852-15; O43852-11; | | |
| O43852-8; O43852-10; O43852-6 | | |
| P04040 | Catalase | CAT |
| Q06830; Q13162 | Peroxiredoxin-1; Peroxiredoxin-4 | PRDX1; PRDX4 |
| P69905 | Hemoglobin subunit alpha | HBA1 |
| Q13404-8; Q15819; Q13404; | Ubiquitin-conjugating enzyme E2 variant 1; | UBE2V1; UBE2V2 |
| Q13404-7; Q13404-2; Q13404-1 | Ubiquitin-conjugating enzyme E2 variant 2 | |
| P06703 | Protein S100-A6 | S100A6 |
| P60174-1; P60174; P60174-4 | Triosephosphate isomerase | TPI1 |
| P62937; P62937-2 | Peptidyl-prolyl cis-trans isomerase A; Peptidyl- | PPIA |
| | prolyl cis-trans isomerase A, N-terminally processed | |
| P49746-2; P49746 | Thrombospondin-3 | THBS3 |
| P09486 | SPARC | SPARC |
| Q14766-3; Q14766-2; Q14766-5; | Latent-transforming growth factor beta-binding | LTBP1 |
| Q14766; Q14766-4 | protein 1 | |
| P01137 | Transforming growth factor beta-1; Latency- | TGFB1 |
| | associated peptide | |
| P61088; Q5JXB2 | Ubiquitin-conjugating enzyme E2 N; Putative | UBE2N; UBE2NL |
| | ubiquitin-conjugating enzyme E2 N-like | |
| P00558-2; P00558 | Phosphoglycerate kinase 1 | PGK1 |
| P07996; P07996-2 | Thrombospondin-1 | THBS1 |
| P02776 | Platelet factor 4; Platelet factor 4, short form | PF4 |
| P0DJI8; P0DJI9-2 | Serum amyloid A-1 protein; Amyloid protein A; | SAA1; SAA2 |
| | Serum amyloid protein A(2-104); Serum amyloid | |
| | protein A(3-104); Serum amyloid protein A(2-103); | |
| | Serum amyloid protein A(2-102); Serum amyloid | |
| | protein A(4-101); Serum amyloid A-2 protein | |
| P10124 | Serglycin | SRGN |
| P13798 | Acylamino-acid-releasing enzyme | APEH |
| P23528 | Cofilin-1 | CFL1 |
| P30041 | Peroxiredoxin-6 | PRDX6 |
| Q8WUM4; Q8WUM4-2 | Programmed cell death 6-interacting protein | PDCD6IP |
| P05090 | Apolipoprotein D | APOD |
| Q99497 | Protein deglycase DJ-1 | PARK7 |
| P35579; P35579-2 | Myosin-9 | MYH9 |
| Q13228; Q13228-4; Q13228-2; | Selenium-binding protein 1 | SELENBP1 |
| Q13228-3 | | |
| P13716; P13716-2 | Delta-aminolevulinic acid dehydratase | ALAD |
| Q9UL13-2; Q9UL13 | Protein HEG homolog 1 | HEG1 |
| P07384 | Calpain-1 catalytic subunit | CAPN1 |
| P18065 | Insulin-like growth factor-binding protein 2 | IGFBP2 |
| P04083 | Annexin A1 | ANXA1 |
| P0DMV8-2; P0DMV9; P0DMV8 | Heat shock 70 kDa protein 1A; Heat shock 70 | HSPA1A; HSPA1B |
| | kDa protein 1B | |
| P07911-3; P07911; P07911-5; | Uromodulin; Uromodulin, secreted form | UMOD |
| P07911-4 | | |
| P15924; P15924-2; P15924-3 | Desmoplakin | DSP |

Group 1B

| UniProt ID | Protein name | Gene name |
|---|---|---|
| Q9C0H2-3 | Protein tweety homolog 3 | TTYH3 |

Group 2A

| | | Entrez | Entrez |
|---|---|---|---|
| Protein name | UniProt ID | Gene ID | Gene Symbol |
| Pulmonary surfactant-associated protein D | P35247 | 6441 | SFTPD |
| Histone H1.2 | P16403 | 3006 | HIST1H1C |
| Sialic acid-binding Ig-like lectin 9 | Q9Y336 | 27180 | SIGLEC9 |
| Heterogeneous nuclear ribonucleoproteins A2/B1 | P22626 | 3181 | HNRNPA2B1 |
| Hexokinase-2 | P52789 | 3099 | HK2 |
| High mobility group protein B1 | P09429 | 3146 | HMGB1 |
| Thrombospondin-1 | P07996 | 7057 | THBS1 |
| 3-hydroxy-3-methylglutaryl-coenzyme A reductase | P04035 | 3156 | HMGCR |
| Platelet factor 4 | P02776 | 5196 | PF4 |
| Protein S100-A9 | P06702 | 6280 | S100A9 |
| 40S ribosomal protein S3a | P61247 | 6189 | RPS3A |
| Annexin A6 | P08133 | 309 | ANXA6 |
| Inosine-5′-monophosphate dehydrogenase 1 | P20839 | 3614 | IMPDH1 |
| Tyrosine-protein kinase Fgr | P09769 | 2268 | FGR |
| Serine/threonine-protein kinase 17B | O94768 | 9262 | STK17B |
| Neutrophil-activating peptide 2 | P02775 | 5473 | PPBP |
| Estradiol 17-beta-dehydrogenase 1 | P14061 | 3292 | HSD17B1 |
| Connective tissue-activating peptide III | P02775 | 5473 | PPBP |
| Prostaglandin G/H synthase 2 | P35354 | 5743 | PTGS2 |
| Amyloid beta A4 protein | P05067 | 351 | APP |
| GTP-binding nuclear protein Ran | P62826 | 5901 | RAN |
| NAD-dependent protein deacetylase sirtuin-2 | Q8IXJ6 | 22933 | SIRT2 |
| Protein S100-A12 | P80511 | 6283 | S100A12 |
| Plasminogen activator inhibitor 1 | P05121 | 5054 | SERPINE1 |
| SUMO-conjugating enzyme UBC9 | P63279 | 7329 | UBE2I |
| Bactericidal permeability-increasing protein | P17213 | 671 | BPI |
| 6-phosphogluconate dehydrogenase, decarboxylating | P52209 | 5226 | PGD |
| Platelet-derived growth factor subunit B | P01127 | 5155 | PDGFB |
| Tyrosine-protein phosphatase non-receptor type 6 | P29350 | 5777 | PTPN6 |
| Metalloproteinase inhibitor 3 | P35625 | 7078 | TIMP3 |
| NudC domain-containing protein 3 | Q8IVD9 | 23386 | NUDCD3 |
| SPARC | P09486 | 6678 | SPARC |
| Protein 4.1 | P11171 | 2035 | EPB41 |
| Sex hormone-binding globulin | P04278 | 6462 | SHBG |
| Death-associated protein kinase 2 | Q9UIK4 | 23604 | DAPK2 |
| Hepatoma-derived growth factor-related protein 2 | Q7Z4V5 | 84717 | HDGFRP2 |
| Proto-oncogene vav | P15498 | 7409 | VAV1 |
| Serine/threonine-protein kinase pim-1 | P11309 | 5292 | PIM1 |
| Heterogeneous nuclear ribonucleoprotein A/B | Q99729 | 3182 | HNRNPAB |
| Proliferation-associated protein 2G4 | Q9UQ80 | 5036 | PA2G4 |
| Extracellular superoxide dismutase [Cu—Zn] | P08294 | 6649 | SOD3 |
| Angiopoietin-1 | Q15389 | 284 | ANGPT1 |
| Acid sphingomyelinase-like phosphodiesterase 3a | Q92484 | 10924 | SMPDL3A |
| Peroxiredoxin-6 | P30041 | 9588 | PRDX6 |
| AMP Kinase (alpha1beta1gamma1) | Q13131 | 5562 5564 5571 | PRKAA1 |
| | Q9Y478 | | PRKAB1 |
| | P54619 | | PRKAG1 |
| Serum amyloid A-1 protein | P0DJI8 | 6288 | SAA1 |
| Insulin-like growth factor-binding protein 2 | P18065 | 3485 | IGFBP2 |
| Histone H2B type 2-E | Q16778 | 8349 | HIST2H2BE |
| Fibroblast growth factor 5 | P12034 | 2250 | FGF5 |
| Non-histone chromosomal protein HMG-14 | P05114 | 3150 | HMGN1 |
| Calcium/calmodulin-dependent protein kinase type | Q13557 | 817 | CAMK2D |
| II subunit delta | | | |
| Interstitial collagenase | P03956 | 4312 | MMP1 |
| ICOS ligand | O75144 | 23308 | ICOSLG |
| Calcium/calmodulin-dependent protein kinase type | Q13554 | 816 | CAMK2B |
| II subunit beta | | | |
| Inosine-5′-monophosphate dehydrogenase 2 | P12268 | 3615 | IMPDH2 |
| Dickkopf-related protein 4 | Q9UBT3 | 27121 | DKK4 |
| Glia-derived nexin | P07093 | 5270 | SERPINE2 |
| 14-3-3 protein beta/alpha | P31946 | 7529 | YWHAB |
| Macrophage-capping protein | P40121 | 822 | CAPG |
| ADP-ribosyl cyclase/cyclic ADP-ribose hydrolase 1 | P28907 | 952 | CD38 |
| SH2 domain-containing protein 1A | O60880 | 4068 | SH2D1A |
| Vacuolar protein sorting-associated protein VTA1 | Q9NP79 | 51534 | VTA1 |
| homolog | | | |
| Interleukin-1 beta | P01584 | 3553 | IL1B |
| Chloride intracellular channel protein 1 | O00299 | 1192 | CLIC1 |
| MAP kinase-activated protein kinase 3 | Q16644 | 7867 | MAPKAPK3 |
| Mitogen-activated protein kinase 1 | P28482 | 5594 | MAPK1 |
| Choline/ethanolamine kinase | Q9Y259 | 1120 | CHKB |
| Creatine kinase B-type | P12277 | 1152 | CKB |
| DNA topoisomerase 1 | P11387 | 7150 | TOP1 |
| C-C motif chemokine 5 | P13501 | 6352 | CCL5 |
| C3a anaphylatoxin | P01024 | 718 | C3 |
| Copine-1 | Q99829 | 8904 | CPNE1 |
| Chymase | P23946 | 1215 | CMA1 |
| Mitogen-activated protein kinase kinase kinase 7: | O43318 Q15750 | 6885 10454 | MAP3K7 TAB1 |
| TGF-beta-activated kinase 1 and | | | |
| MAP3K7-binding protein 1 fusion | | | |
| Dickkopf-related protein 1 | O94907 | 22943 | DKK1 |
| Ephrin type-B receptor 4 | P54760 | 2050 | EPHB4 |
| 40S ribosomal protein S7 | P62081 | 6201 | RPS7 |
| Intercellular adhesion molecule 1 | P05362 | 3383 | ICAM1 |
| Ubiquitin + 1, truncated mutation for UbB | P62979 | 6233 | RPS27A |
| Ubiquitin-conjugating enzyme E2 N | P61088 | 7334 | UBE2N |
| Moesin | P26038 | 4478 | MSN |
| Small ubiquitin-related modifier 3 | P55854 | 6613 | SUMO3 |
| G2/mitotic-specific cyclin-B1 | P14635 | 891 | CCNB1 |
| ATP-dependent RNA helicase DDX19B | Q9UMR2 | 11269 | DDX19B |
| Tyrosine-protein kinase CSK | P41240 | 1445 | CSK |
| Alpha-1-antitrypsin | P01009 | 5265 | SERPINA1 |
| Adenylate kinase isoenzyme 1 | P00568 | 203 | AK1 |
| Mothers against decapentaplegic homolog 3 | P84022 | 4088 | SMAD3 |
| Brain-derived neurotrophic factor | P23560 | 627 | BDNF |
| Atrial natriuretic factor | P01160 | 4878 | NPPA |
| Annexin A1 | P04083 | 301 | ANXA1 |
| Signal transducer and activator of transcription | P42224 | 6772 | STAT1 |
| 1-alpha/beta | | | |
| Angiopoietin-related protein 4 | Q9BY76 | 51129 | ANGPTL4 |
| Cadherin-12 | P55289 | 1010 | CDH12 |
| Stress-induced-phosphoprotein 1 | P31948 | 10963 | STIP1 |
| Glycylpeptide N-tetradecanoyltransferase 1 | P30419 | 4836 | NMT1 |
| Peroxiredoxin-1 | Q06830 | 5052 | PRDX1 |
| Oxidized low-density lipoprotein receptor 1 | P78380 | 4973 | OLR1 |
| VPS10 domain-containing receptor SorCS2 | Q96PQ0 | 57537 | SORCS2 |
| FACT complex subunit SSRP1 | Q08945 | 6749 | SSRP1 |
| Carbonic anhydrase 1 | P00915 | 759 | CA1 |
| Mitogen-activated protein kinase 11 | Q15759 | 5600 | MAPK11 |
| Triosephosphate isomerase | P60174 | 7167 | TPI1 |
| Neurexophilin-1 | P58417 | 30010 | NXPH1 |
| Platelet-derived growth factor subunit A | P04085 | 5154 | PDGFA |
| lnterleukin-22 receptor subunit alpha-1 | Q8N6P7 | 58985 | IL22RA1 |

Group 2B

| | | Entrez | Entrez |
|---|---|---|---|
| Protein name | UniProt ID | Gene ID | Gene Symbol |
| Cystatin-SN | P01037 | 1469 | CST1 |
| Interleukin-22 | Q9GZX6 | 50616 | IL22 |
| Tyrosine-protein kinase Fyn | P06241 | 2534 | FYN |
| Biglycan | P21810 | 633 | BGN |
| Sonic hedgehog protein | Q15465 | 6469 | SHH |
| Cathepsin L2 | O60911 | 1515 | CTSV |
| Interferon alpha/beta receptor 1 | P17181 | 3454 | IFNAR1 |
| Ectodysplasin-A, secreted form | Q92838 | 1896 | EDA |
| Dynein light chain 1, cytoplasmic | P63167 | 8655 | DYNLL1 |
| Cytochrome c | P99999 | 54205 | CYCS |
| Lactoperoxidase | P22079 | 4025 | LPO |
| Tumor necrosis factor receptor | Q969Z4 | 84957 | RELT |
| superfamily member 19L | | | |
| Growth factor receptor-bound protein 2 | P62993 | 2885 | GRB2 |
| Ectonucleotide pyrophosphatase/ | Q6UWV6 | 339221 | ENPP7 |
| phosphodiesterase family member 7 | | | |
| Glucagon | P01275 | 2641 | GCG |
| Eukaryotic translation initiation factor 4 | P78344 | 1982 | EIF4G2 |
| gamma 2 | | | |
| C-X-C motif chemokine 10 | P02778 | 3627 | CXCL10 |
| Platelet-derived growth factor receptor | P09619 | 5159 | PDGFRB |
| beta | | | |
| von Willebrand factor | P04275 | 7450 | VWF |
| Iduronate 2-sulfatase | P22304 | 3423 | IDS |
| cGMP-specific 3′,5′-cyclic | O76074 | 8654 | PDE5A |
| phosphodiesterase | | | |
| Small glutamine-rich tetratricopeptide | O43765 | 6449 | SGTA |
| repeat-containing protein alpha | | | |
| Interleukin-25 | Q9H293 | 64806 | IL25 |
| MHC class I polypeptide-related | Q29980 | 4277 | MICB |
| sequence B | | | |
| C-C motif chemokine 7 | P80098 | 6354 | CCL7 |
| Lactadherin | Q08431 | 4240 | MFGE8 |
| Macrophage metalloelastase | P39900 | 4321 | MMP12 |
| Ubiquitin-like protein ISG15 | P05161 | 9636 | ISG15 |
| Fatty acid-binding protein, liver | P07148 | 2168 | FABP1 |
| Properdin | P27918 | 5199 | CFP |
| Stromelysin-2 | P09238 | 4319 | MMP10 |
| Protein FAM107B | Q9H098 | 83641 | FAM107B |
| Myosin-binding protein C, slow-type | Q00872 | 4604 | MYBPC1 |
| Serine/threonine-protein kinase Chk2 | O96017 | 11200 | CHEK2 |
| Xaa-Pro aminopeptidase 1 | Q9NQW7 | 7511 | XPNPEP1 |
| NT-3 growth factor receptor | Q16288 | 4916 | NTRK3 |
| Interleukin-5 receptor subunit alpha | Q01344 | 3568 | IL5RA |
| Ephrin type-A receptor 2 | P29317 | 1969 | EPHA2 |
| Peptidyl-prolyl cis-trans isomerase F, | P30405 | 10105 | PPIF |
| mitochondrial | | | |
| B-cell lymphoma 6 protein | P41182 | 604 | BCL6 |
| Cell adhesion molecule 1 | Q9BY67 | 23705 | CADM1 |
| Kinesin-like protein KIF23 | Q02241 | 9493 | KIF23 |
| Netrin receptor UNC5D | Q6UXZ4 | 137970 | UNC5D |
| Lymphocyte activation gene 3 protein | P18627 | 3902 | LAG3 |
| Serine protease HTRA2, mitochondrial | O43464 | 27429 | HTRA2 |
| Interleukin-22 receptor subunit alpha-2 | Q969J5 | 116379 | IL22RA2 |
| Caspase-3 | P42574 | 836 | CASP3 |
| Platelet-derived growth factor receptor | P16234 | 5156 | PDGFRA |
| alpha | | | |
| Interleukin-20 | Q9NYY1 | 50604 | IL20 |
| Ferritin | P02794 P02792 | 2495 2512 | FTH1 FTL |
| Ephrin-A5 | P52803 | 1946 | EFNA5 |
| Amphoterin-induced protein 2 | Q86SJ2 | 347902 | AMIGO2 |
| Interleukin-2 | P60568 | 3558 | IL2 |
| Phospholipase A2 | P04054 | 5319 | PLA2G1B |
| Protein kinase C alpha type | P17252 | 5578 | PRKCA |
| Tumor necrosis factor ligand | P32971 | 944 | TNFSF8 |
| superfamily member 8 | | | |
| Endoglin | P17813 | 2022 | ENG |
| Gamma-enolase | P09104 | 2026 | ENO2 |
| C-X-C motif chemokine 9 | Q07325 | 4283 | CXCL9 |
| beta-nerve growth factor | P01138 | 4803 | NGF |
| Hepcidin | P81172 | 57817 | HAMP |
| Inorganic pyrophosphatase | Q15181 | 5464 | PPA1 |
| Kallikrein-8 | O60269 | 11202 | KLK8 |
| Semaphorin-6B | Q9H3T3 | 10501 | SEMA6B |
| Adapter molecule crk | P46108 | 1398 | CRK |
| Phosphoglycerate mutase 1 | P18669 | 5223 | PGAM1 |
| Proprotein convertase subtilisin/ | Q16549 | 9159 | PCSK7 |
| kexin type 7 | | | |
| Pancreatic hormone | P01298 | 5539 | PPY |
| Interleukin-1 receptor type 1 | P14778 | 3554 | IL1R1 |
| C-X-C motif chemokine 11 | O14625 | 6373 | CXCL11 |
| Secreted frizzled-related protein 1 | Q8N474 | 6422 | SFRP1 |
| Inhibin beta A chain | P08476 | 3624 | INHBA |
| Immunoglobulin D | P01880 | 3495 50802 3535 | IGHD IGK@ |
| | | | IGL@ |
| Complement C3b | P01024 | 718 | C3 |
| Teratocarcinoma-derived growth | P13385 | 6997 | TDGF1 |
| factor 1 | | | |
| Brother of CDO | Q9BWV1 | 91653 | BOC |
| CD109 antigen | Q6YHK3 | 135228 | CD109 |
| Aminoacylase-1 | Q03154 | 95 | ACY1 |
| 60 kDa heat shock protein, | P10809 | 3329 | HSPD1 |
| mitochondrial | | | |
| Importin subunit beta-1 | Q14974 | 3837 | KPNB1 |
| C-reactive protein | P02741 | 1401 | CRP |
| Ephrin type-A receptor 1 | P21709 | 2041 | EPHA1 |
| Semaphorin-6A | Q9H2E6 | 57556 | SEMA6A |
| SLIT and NTRK-like protein 5 | O94991 | 26050 | SLITRK5 |
| Leucine-rich repeat transmembrane | Q9NZU0 | 23767 | FLRT3 |
| protein FLRT3 | | | |
| dCTP pyrophosphatase 1 | Q9H773 | 79077 | DCTPP1 |
| Osteopontin | P10451 | 6696 | SPP1 |
| Formimidoyltransferase-cyclodeaminase | O95954 | 10841 | FTCD |
| Ephrin-B2 | P52799 | 1948 | EFNB2 |
| T-lymphocyte surface antigen Ly-9 | Q9HBG7 | 4063 | LY9 |
| Sialic acid-binding Ig-like lectin 14 | Q08ET2 | 100049587 | SIGLEC14 |
| N-acylethanolamine-hydrolyzing acid | Q02083 | 27163 | NAAA |
| amidase | | | |
| Endoplasmic reticulum aminopeptidase 1 | Q9NZ08 | 51752 | ERAP1 |
| Low affinity immunoglobulin gamma Fc | P12318 | 2212 | FCGR2A |
| region receptor II-a | | | |

Group 3A

| UniProt ID | Protein name | Gene name |
|---|---|---|
| Q15389 | Angiopoietin-1 (ANG-1) | ANGPT1 |
| P23560 | Brain-Derived Neurotrophic Factor (BDNF) | BDNF |
| O94907 | Dickkopf-related protein 1 (DKK-1) | DKK1 |
| P80511 | S100-A12 | S100A12 |
| P42830 | Epithelial-Derived Neutrophil-Activating | CXCL5 |
| | Protein 78 (ENA-78) | |
| P02794 | Ferritin (FRTN) | FTH1 |
| P09341 | Growth-Regulated alpha protein (GRO-alpha) | CXCL1 |
| P01137 | Latency-Associated Peptide of Transforming | TGFB1 |
| | Growth Factor beta 1 (LAP TGF-b1) | |
| Q8WXL0 | Luteinizing Hormone (LH) | LHB |
| G3CBL7 | MHC class I chain-related protein A (MICA) | MICA |
| P02775 | Platelet basic protein | PPBP |
| P01127 | Platelet-Derived Growth Factor BB (PDGF-BB) | PDGFB |
| P01236 | Prolactin (PRL) | PRL |
| P0DJI8; P0DJI9-2 | Serum amyloid A-1 protein; Amyloid protein | SAA1; SAA2 |
| | A; Serum amyloid protein A(2-104); Serum | |
| | amyloid protein A(3-104); Serum amyloid | |
| | protein A(2-103); Serum amyloid protein | |
| | A(2-102); Serum amyloid protein A(4- | |
| | 101); Serum amyloid A-2 protein | |
| P13501 | T-Cell-Specific Protein RANTES (RANTES) | CCL5 |

Group 3B

| UniProt ID | Protein name | Gene name |
|---|---|---|
| P10645 | Chromogranin-A (CgA) | CHGA |
| Q9GZV9 | Fibroblast growth factor 23 (FGF-23) | FGF23 |
| P01266 | Thyroglobulin (TG) | TG |

Group 4A

| UniProt ID | Protein name | Gene name |
|---|---|---|
| P07996 | Thrombospondin-1 OS = Homo sapiens | THBS1 |
| | OX = 9606 GN = THBS1 PE = 1 SV = 2 | |
| P69905 | Hemoglobin subunit alpha OS = Homo sapiens | HBA1 |
| | OX = 9606 GN = HBA1 PE = 1 SV = 2 | |
| P32119 | Peroxiredoxin-2 OS = Homo sapiens | PRDX2 |
| | OX = 9606 GN = PRDX2 PE = 1 SV = 5 | |
| P00918 | Carbonic anhydrase 2 OS = Homo sapiens | CA2 |
| | OX = 9606 GN = CA2 PE = 1 SV = 2 | |
| P30043 | Flavin reductase (NADPH) OS = Homo sapiens | BLVRB |
| | OX = 9606 GN = BLVRB PE = 1 SV = 3 | |
| O76011 | Keratin, type I cuticular Ha4 OS = Homo sapiens | KRT34 |
| | OX = 9606 GN = KRT34 PE = 1 SV = 2 | |
| P13716 | Delta-aminolevulinic acid dehydratase OS = | ALAD |
| | Homo sapiens OX = 9606 GN = ALAD PE = 1 SV = 1 | |
| P00568 | Adenylate kinase isoenzyme 1 OS = Homo sapiens | AK1 |
| | OX = 9606 GN = AK1 PE = 1 SV = 3 | |
| P07738 | Bisphosphoglycerate mutase OS = Homo sapiens | BPGM |
| | OX = 9606 GN = BPGM PE = 1 SV = 2 | |
| P02775 | Platelet basic protein OS = Homo sapiens | PPBP |
| | OX = 9606 GN = PPBP PE = 1 SV = 3 | |
| P22392 | Nucleoside diphosphate kinase B OS = | NME2 |
| | Homo sapiens OX = 9606 GN = NME2 PE = 1 SV = 1 | |
| P10124 | Serglycin OS = Homo sapiens | SRGN |
| | OX = 9606 GN = SRGN PE = 1 SV = 3 | |
| A0A0C4DH67 | Immunoglobulin kappa variable 1-8 OS = | IGKV1-8 |
| | Homo sapiens OX = 9606 GN = IGKV1-8 PE = 3 SV = 1 | |
| P63241 | Eukaryotic translation initiation factor 5A-1 OS = | EIF5A |
| | Homo sapiens OX = 9606 GN = EIF5A PE = 1 SV = 2 | |
| P02776 | Platelet factor 4 OS = Homo sapiens | PF4 |
| | OX = 9606 GN = PF4 PE = 1 SV = 2 | |
| A0A075B6S2 | Immunoglobulin kappa variable 2D-29 OS = | IGKV2D-29 |
| | Homo sapiens OX = 9606 GN = IGKV2D-29 PE = 3 SV = 1 | |
| Q9UKU6 | Thyrotropin-releasing hormone-degrading ectoenzyme | TRHDE |
| | OS = Homo sapiens OX = 9606 GN = TRHDE PE = 2 SV = 1 | |
| Q8WUM4 | Programmed cell death 6-interacting protein OS = | PDCD6IP |
| | Homo sapiens OX = 9606 GN = PDCD6IP PE = 1 SV = 1 | |
| P00441 | Superoxide dismutase [Cu—Zn] OS = Homo sapiens | SOD1 |
| | OX = 9606 GN = SOD1 PE = 1 SV = 2 | |

Group 4B

| UniProt ID | Protein name | Gene name |
|---|---|---|
| P36980 | Complement factor H-related protein 2 OS = Homo sapiens | CFHR2 |
| | OX = 9606 GN = CFHR2 PE = 1 SV = 1 | |
| P0D0X3 | Immunoglobulin delta heavy chain OS = Homo sapiens | N/A |
| | OX = 9606 PE = 1 SV = 1 | |
| P01880 | Immunoglobulin heavy constant delta OS = Homo sapiens | IGHD |
| | OX = 9606 GN = IGHD PE = 1 SV = 3 | |
| Q8N6C8 | Leukocyte immunoglobulin-like receptor subfamily A member | LILRA3 |
| | 3 OS = Homo sapiens OX = 9606 GN = LILRA3 PE = 1 SV = 3 | |
| P47929 | Galectin-7 OS = Homo sapiens | LGALS7 |
| | OX = 9606 GN = LGALS7 PE = 1 SV = 2 | |
| O75339 | Cartilage intermediate layer protein 1 OS = Homo sapiens | CILP |
| | OX = 9606 GN = CILP PE = 1 SV = 4 | |
| O15389 | Sialic acid-binding Ig-like lectin 5 OS = Homo sapiens | SIGLEC5 |
| | OX = 9606 GN = SIGLEC5 PE = 1 SV = 1 | |
| P35247 | Pulmonary surfactant-associated protein D OS = | SFTPD |
| | Homo sapiens OX = 9606 GN = SFTPD PE = 1 SV = 3 | |

2. A method of determining whether or not a subject is affected by endometriosis, comprising measuring the abundance of at least one marker selected from the markers set forth in claim 1 in a sample obtained from the subject.

3. A method of determining the extent of endometrial fibrosis in a subject, comprising measuring the abundance of at least one marker selected from the markers set forth in claim 1 in a sample obtained from the subject.

4. A method of determining the extent of endometrial adhesion in a subject, comprising measuring the abundance of at least one marker selected from the markers set forth in claim 1 in a sample obtained from the subject.

5. A method of predicting the degree of pain due to endometriosis in a subject affected or suspected of being affected by endometriosis, comprising measuring the abundance of at least one marker selected from the markers set forth in claim 1 in a sample obtained from the subject.

6. A method of determining the degree of progression of endometriosis in a subject affected or suspected of being affected by endometriosis, comprising measuring the abundance of at least one marker selected from the markers set forth in claim 1 in a sample obtained from the subject.

7. A method of monitoring pathological conditions of endometriosis in a subject affected or suspected of being affected by endometriosis, comprising measuring each of the abundance of at least one marker selected from the markers set forth in claim 1 in each of a plurality of samples collected from the subject at different points of time.

8. The method of any one of claims 1 to 7, wherein the abundance of the marker is a concentration of a polypeptide of the marker in a blood sample.

9. A kit for diagnosing endometriosis, comprising a reagent for measuring the abundance of at least one marker selected from the markers set forth in claim 1.

10. A kit for determining whether or not a subject is affected by endometriosis, comprising a reagent for measuring the abundance of at least one marker selected from the markers set forth in claim 1.

11. A kit for determining the extent of endometrial fibrosis in a subject, comprising a reagent for measuring the abundance of at least one marker selected from the markers set forth in claim 1.

12. A kit for determining the extent of endometrial adhesion in a subject, comprising a reagent for measuring the abundance of at least one marker selected from the markers set forth in claim 1.

13. A kit for predicting the degree of pain due to endometriosis in a subject affected or suspected of being affected by endometriosis, comprising a reagent for measuring the abundance of at least one marker selected from the markers set forth in claim 1.

14. A kit for determining the degree of progression of endometriosis in a subject affected or suspected of being affected by endometriosis, comprising a reagent for measuring the abundance of at least one marker selected from the markers set forth in claim 1.

15. A kit for monitoring pathological conditions of endometriosis in a subject affected or suspected of being affected by endometriosis, comprising a reagent for measuring the abundance of at least one marker selected from the markers set forth in claim 1, and instructions stating that the abundance of at least one marker selected from the markers set forth in claim 1 in each of a plurality of samples collected from the subject at different points of time is compared.
